(19) United States
(12) Patent Application Publication
(10) Pub. No.: US 2023/0085357 A1
(43) Pub. Date: Mar. 16, 2023

(54) INJECTABLE HIGH CONCENTRATION PHARMACEUTICAL FORMULATIONS AND METHODS OF MANUFACTURING AND USE THEREOF

(71) Applicant: Xeris Pharmaceuticals, Inc., Chicago, IL (US)

(72) Inventors: Martin DONOVAN, El Paso, TX (US); Steven PRESTRELSKI, San Diego, CA (US); Scott COLEMAN, San Diego, CA (US); Brian SLOAT, Austin, TX (US); Diana BOWMAN, Chicago, IL (US); Richard FITCH, Chicago, IL (US)

(73) Assignee: Xeris Pharmaceuticals, Inc., Chicago, IL (US)

(21) Appl. No.: 17/930,857

(22) Filed: Sep. 9, 2022

Related U.S. Application Data
(60) Provisional application No. 63/351,786, filed on Jun. 13, 2022.
(60) Provisional application No. 63/242,405, filed on Sep. 9, 2021.

(51) Int. Cl. A61K 9/06; A61K 47/26; A61K 47/12; A61K 47/44; C07K 16/32; C07K 16/28; C07K 16/24; A61K 38/26; A61K 38/28; A61K 38/46; A61K 38/47; A61K 38/50; A61K 38/48; A61K 38/58; A61K 31/7088; A61K 31/7076; A61K 38/49; A61K 38/35; A61K 38/24; A61K 38/18; A61K 38/23; A61K 31/137; A61K 31/4045; A61K 31/136; A61K 31/337; A61K 31/5513; A61K 9/00; C07K 16/22; A61K 38/44

(52) U.S. Cl. CPC A61K 9/06; A61K 47/26; A61K 47/12; A61K 47/44; C07K 16/32; C07K 16/2818; C07K 16/241; C07K 16/2875; C07K 16/2839; C07K 16/2863; A61K 38/26; A61K 38/28; A61K 38/465; A61K 38/47; A61K 38/50; A61K 38/4813; C12Y 301/01013; C12Y 302/01; C12Y 107/03003; A61K 38/58; A61K 31/7088; A61K 31/7076; A61K 38/482; C12Y 304/00; A61K 38/49; A61K 38/35; A61K 38/24; A61K 38/18; A61K 38/23; A61K 31/137; A61K 31/4045; A61K 31/136; A61K 31/337; A61K 31/5513; A61K 9/0019; C07K 16/22; C12Y 301/21001; C12Y 302/01045; C12Y 305/01001; C12Y 304/17011; C12Y 301/06004; A61K 38/44; C12Y 304/21068; C12Y 304/21073; C07K 2317/90; C07K 2317/24; C07K 2317/21

(57) ABSTRACT

The present invention provides compositions comprising one or more active pharmaceutical ingredients, wherein the compositions are in the form of high solids concentration pastes capable of being injected in relatively low volumes into an animal using standard commercially available syringes. The invention also provides methods of making such compositions, particularly those compositions comprising high molecular weight active ingredients (e.g., antibodies, enzymes and other proteins and peptides) at relatively high therapeutic concentrations in the high solids concentration pastes. The invention further provides methods of using such formulations in treating, preventing and/or ameliorating certain diseases and physical disorders in animals, including humans, in need thereof. The invention also provides kits comprising the formulations of the invention and a suitable syringe, which in some aspects may be pre-loaded or pre-filled with a composition of the invention.

### CROSS REFERENCE TO RELATED APPLICATIONS

This application claims the priority benefit of U.S. Provisional Application No. 63/242,405, filed Sep. 9, 2021, and of U.S. Provisional Application No. 63/351,786, filed Jun. 13, 2022, the disclosures of which are incorporated by reference herein in their entireties.

### STATEMENT REGARDING PRIOR DISCLOSURES BY THE INVENTOR OR A JOINT INVENTOR

Some of the material disclosed herein was disclosed in U.S. Pat. Nos. 8,110,209, 8,790,679 and 9,314,424, and in U.S. Published Application No. 2017/0216529.

### BACKGROUND OF THE INVENTION

### Field of the Invention

The present invention relates generally to parenteral, i.e., intracutaneous, subcutaneous and/or intramuscular, injection of pharmaceutical formulations including those containing at least one active pharmaceutical ingredient at high concentrations, particularly in the form of pastes, and provides such formulations, methods of manufacturing and use of such formulations and kits comprising such formulations.

### Description of Related Art

Parenteral injection refers to the administration of drugs, medications or vaccines via injection under or through one or more layers of skin or mucus membranes of an animal. Standard injections are given into the subcutaneous or intramuscular region of an animal, e.g., a human patient. These deep locations are targeted because the tissue expands more easily, relative to shallow dermal sites, to accommodate the 0.1-3.0 cc (ml) injection volumes required to deliver most therapeutic agents.

Generally, injections have been classified into different categories, including (1) solutions ready for injection; (2) dry soluble products (solutes) ready to be combined with a solvent just prior to being injected into a patient; (3) dry, insoluble products ready to be combined with a suitable injection medium prior to administration; (4) suspensions ready for injection; and (5) emulsions ready for injection. Such injectable formulations are administered by routes including intravenous, subcutaneous, intradermal, intramuscular, intraspinal, intracisternal, and intrathecal. The nature of the therapeutic agent and of the disease or disorder being treated quickly determines the route of administration. However, the desired route of administration places constraints on the therapeutic formulation itself. For example, solutions for subcutaneous administration require strict attention to tonicity adjustment in order to avoid irritation to the nerves and tissue in the surrounding area of injection. Likewise, suspensions are not administered directly into the blood stream in view of the potential of insoluble particles blocking capillaries.

In comparison to other dosage forms and routes of administration (e.g., oral, transdermal), injectables possess certain advantages, including immediate physiological action (e.g., via intravenous injection), avoidance of intestinal absorption problems attended with many drugs, and the accurate administration of the desired dose into the blood stream of a patient. On the other hand, one of the disadvantages of injectables is the pain and discomfort present at the site of administration associated with certain pharmaceutically active agents, as well as the trauma of having a needle inserted under the skin or into a vein. There is a degree of discomfort for the patient with each injection which is administered.

Currently, biopharmaceutical agents are typically reconstituted into sterile solutions and are administered into the subcutaneous or intramuscular space using a large gauge needle, e.g., in the range 18-30 gauge. Pain is caused by the depth of the penetration of the needle, the size “gauge” of the needle, the large volume of injection, and the diffusion of drug away from the site of injection, among other things. In addition to problems with administration of injectables due to pain associated with the same, there are other drawbacks of current practices with respect to injections. For example, many proteins and sustained release drugs require reconstitution immediately prior to administration. Dosing of drugs can be inflexible and inaccurate. Further, many formulations need to be refrigerated to protect the drug(s) from physical and/or chemical degradation (e.g., hydrolysis). Further, current administration systems are wasteful in that the injection device retains a significant amount of the drug product. Further, to effect delivery of the necessary dose required, an injectable formulation typically must be concentrated and stabilized. Standard injections are given in the liquid form. Products that are sold as liquids or a lyophilized powder require reconstitution in an aqueous carrier prior to injection. Many therapeutic protein and vaccine products are produced in a dry, solid form to promote stability while on the shelf. These formulations are diluted/reconstituted to a solution or suspension prior to injection in a pharmaceutically acceptable medium, including sterile water for injection (SWFI), phosphate buffer solution, or isotonic saline.

More recently, the preparation and use of high concentration pharmaceutical formulations in the form of low-moisture suspensions, colloids or pastes, has been described (see, e.g., U.S. Pat. Nos. 8,110,209, 8,790,679, and 9,314,424, and US Patent Publication No. 2017/0216529, the disclosures of all of which are incorporated herein by reference in their entireties). Such formulations contain active pharmaceutical ingredients at substantially higher concentrations than found in traditional aqueous pharmaceutical formulations. A paste, or a two-phase mixture of a solid dispersed in a non-solvent liquid (e.g., relative to the solid), can be an effective dosage form? for delivering medicament(s) (e.g., intracutaneously). For example, pastes may be able to achieve much higher solids (e.g., drug) concentrations than typical solutions (e.g., water-based solutions) while also providing greater stability relative to aqueous solutions as the active ingredient in a paste may be formulated in the solid state (e.g., as a powder). This approach can be particularly advantageous for formulating active pharmaceutical ingredients that are not very soluble in aqueous solutions or that are prone to chemical degradation (e.g., hydrolysis), and/or physical instability (e.g., aggregation) upon being formulated into a low-concentration highly aqueous formulation for delivery to a patient.

Pastes are semi-solid dosage forms containing a high percentage of finely dispersed solids (e.g., powder particles) in an oleaginous material (e.g., oils or hydrocarbon bases) with a relatively stiff and thick consistency. The actual solids content (or solids concentration—in both cases describing the amount of solids in the formulation, with ‘solids content’ expressing the weight-percent of the solids relative to the total weight of the formulation (solids plus liquid), while ‘solids concentration’ expresses the concentration of solid per unit volume (e.g., g/mL, mg/mL, etc.) in the formulation) of the paste will primarily depend on the properties of the constituent powder, and can range both below and above that provided in the USP-NF definition (see, e.g., U.S. Pat. Nos. 8,110,209, 8,790,679, 9,314,424, and 11,129,940, the disclosures of all of which are incorporated herein by reference in their entireties). To prepare a paste, the minimum quantity of fluid that is added to a powder must be sufficient to coat the powder particles. In an idealized situation all powder-powder contacts have been fully disrupted and each powder particle is not adhered/attached/agglomerated with any other particle. However, in practice many micronized powders are highly cohesive and complete disruption of all direct powder-powder contacts may not be possible despite the application of high-shear mixing techniques. Additional fluid is then added to the mixture to fill in the interstitial spaces between the powder particles (i.e., the void volume) and thus enable the particles to flow as a fluid when the yield stress of the paste has been exceeded. Accordingly, powders possessing very low density (i.e., those having a high surface area-to-volume ratio) will require a greater volume of fluid to form a paste compared to powders with a lower surface area-to-volume ratio. Therefore, as will be discussed further, the percent solids content of a paste can vary greatly and can depend on multiple factors, including the process by which it was prepared (e.g., non-limiting examples include freeze drying, spray drying, spray freeze-drying, thin-film freezing, solvent extraction/exchange, coacervation, and additional particle engineering techniques that are known in the art).

Though being a two-phase system (containing both a solid (particulate) phase dispersed in a liquid (diluent/non-solvent) phase) and thus often falling under the category of suspensions, pastes are physically distinct from traditional suspensions and other formulations with high solids concentration such as gels, in that the concentration of the particulate matter (e.g., powder) in the composition is sufficiently high such that the particles are prevented from settling in the fluid over storage conditions and storage periods relevant to commercial pharmaceutical drug products. This provides pastes with the stiff consistency, relative to gels, creams, foams and other ‘semi-solid’ pharmaceutical dosage forms, that renders pastes highly viscous.

Accordingly, parenteral (e.g., intracutaneous, subcutaneous and/or intramuscular) delivery (e.g., injection) of such pastes may pose difficulties. In particular, such pastes typically have a significantly higher apparent viscosity when compared with traditional aqueous solutions, and it is generally believed that injection of such high viscosity pastes using traditional syringes is difficult, if not impossible (e.g., requiring excessive force and/or causing excessive pain due, for example, the use of large needles). Further, being two-phase mixtures of liquids containing homogeneously dispersed particulate matter, these compositions are particularly susceptible to either partial and/or complete clogging of the delivery device, imposing a further limitation on the potential for intracutaneously delivering therapeutic pastes.

Methods of injecting pastes have been previously disclosed. For example, U.S. Pat. Nos. 8,790,679, 8,110,209 and 9,314,424, and U.S. Patent Publication Nos. US 2017/0007675 and US 2017/0216529 (the disclosures of all of which are incorporated herein by reference in their entireties) disclose the preparation of therapeutic pastes for intracutaneous administration and indicate that because paste formulations typically display poor flow properties in standard syringes, novel needle/syringe designs are required to deliver such formulations. To accomplish delivery, the injection device preferably incorporates a plunger that can fit into the lumen of the needle, and that acts in a way such that the full amount of the therapeutic formulation loaded into the device is loaded into the lumen of the needle and is then pushed out into the patient upon administration using a positive displacement design. Notably, however, this type of configuration would require a plunger that fits within the lumen of a needle and that is displaced toward the end of the needle upon activation in such a manner that substantially all (e.g., approaching or equal to 100%) of the loaded therapeutic formulation is dispensed through the needle and into the location of injection.

As is well known in the field, commercially available syringes possess internal barrel diameters that are several times larger than the internal diameter of the lumen of a needle. For example, the standard 1-mL long syringes used in many commercial injectable drug products have an internal diameter of approximately 6.4 mm (compared to approximately 0.26 mm for a 25 G needle). Moreover, the injection device described in the prior art would only be capable of delivering a very small volume of paste and/or fluid through a standard needle. As an example, a typical needle used for subcutaneous injection is a 27-gauge (or 27 G), ultra-thin wall (UTW) 6-mm long needle. This needle has an internal diameter of approximately 300 μm (0.300 mm). Modeling the internal volume of the needle as a cylinder of height 6 mm and diameter 0.300 mm, the volume of paste that can be contained within such a needle is 4.24×10⁻⁴ cm³, or approximately 0.42 μL. Typical injection volumes for intracutaneous delivery often range from 100-1000 μL (0.1-1.0 mL), and depending on the indication, drug, etc., the delivered volume may be even larger (e.g., 2000 or 3000 μL). Thus, delivery of most therapeutically relevant volumes will require very long and very large (with respect to the internal diameter) needles.

As is further discussed in the art, “the needle portion of the injection device is from about 6 to about 8 cm in length, thereby providing a lumen having a sufficient interior volume to contain the dose of semi-solid therapeutic formulation and the plunger.” US Patent Publication 2006/0211982, paragraph [0115]. Typical needle lengths for intradermal (I.D.) and subcutaneous (S.C.) administration are ≥0.5 inches (or 1.3 cm). Even deeper intramuscular (I.M.) injections commonly employ needles only between 1.0 and 1.5 inches (or between 2.5-3.8 cm). Accordingly, the needles envisioned for the administration of viscous therapeutic pastes would have to be at least twice as long as commercially available needles. However, even using these long and specially designed needles, and also assuming a relatively large internal diameter, the volume that can be placed within the lumen may still be well below that required to achieve a therapeutic dose. For example, the internal volume of an 8-cm long, 18 G needle (internal diameter of 0.84 mm) is only 4.4×10⁻² cm³, or approximately 44 μL.

In addition to the small volumes that can be administered from an arrangement where the entire dose is contained within the lumen of the needle, such long needles typically have to be specially manufactured and may be frightening or repulsive to certain patients due to their length. Moreover, as injection pain can be related to the overall diameter (i.e., gauge) of the needle, such large needles may be very painful, and thus adversely affect patient compliance with a dosing regimen that requires multiple injections with such large needle.

Accordingly, there is a need in the art for storage-stable compositions, methods, kits and devices for use in parenteral delivery of a highly concentrated, viscous, non-Newtonian fluids (such as pastes) comprising one or more therapeutic agents at high concentrations, particularly therapeutic agents that are themselves of relatively high molecular weight (for example, biologics including antibodies (monoclonal and/or polyclonal) and fragments or complexes thereof, vaccines, enzymes, receptor agonistic or antagonistic peptides and proteins, oligonucleotides and vectors comprising them, and the like), using standard syringes coupled to needles that are typically used for administration. There is an additional need for compositions, methods, kits and/or devices for delivery of a volume of such therapeutic fluids (including pastes) that may exceed the volume of the lumen of a needle.

### BRIEF SUMMARY OF THE INVENTION

The present invention provides compositions suitable for the parenteral, i.e., intracutaneous, subcutaneous and/or intramuscular administration of high concentration pharmaceutical formulations, and provides such formulations, methods of manufacturing and use of such formulations and kits comprising such formulations. Certain aspects of the invention described herein are directed to the discovery that non-Newtonian fluids and viscoelastic semi-solid compositions such as pastes (and even high viscosity Newtonian fluids) comprising high concentrations of active pharmaceutical ingredients can be readily delivered parenterally from a standard (i.e., commercially available) syringe/needle combination. In this way, the present invention provides pharmaceutical formulations comprising a high mass of an active pharmaceutical ingredient in a relatively low volume of diluent or carrier (compared to traditional aqueous pharmaceutical formulations), particularly wherein the formulations are manufactured in a way that permits administration of the formulation to a patient other than via intravenously, e.g., subcutaneously, intradermally or parenterally, in a way that provides a ready-to-use formulation (i.e., one that does not require reconstitution or dilution prior to being administered to a patient), and that may additionally provide longer-term storage stability than has been previously achieved. The present invention thus facilitates the manufacturing, storage and delivery of medications parenterally which previously were only delivered intravenously—i.e., the ability to take a large volume medication (regardless of the route of administration) and deliver the same therapeutic effect using a smaller volume intracutaneous, subcutaneous or intramuscular injection. In certain embodiments, this approach is coupled with a reduction in adverse injection site reactions that often accompany parenteral injection of high-volume pharmaceutical formulations.

In one aspect, the present invention provides methods of producing high concentration/high viscosity (e.g., formulations having a viscosity above about 50 cP, above about 100 cP, above about 200 cP, or above about 250 cP of apparent viscosity) injectable formulations for parenteral injection of therapeutic agents or active pharmaceutical ingredients, in the form of a high solids content paste. Certain methods according to this aspect of the invention comprise spray-drying and lyophilizing aqueous formulations comprising one or more active pharmaceutical ingredients, and then processing (e.g., grinding, sieving, etc.) the resulting powders to disrupt larger agglomerates and yield powder and powder particles of relatively small diameter and narrow size distribution such that they can be delivered through small diameter needles appropriate for administration by parenteral injection. Such powders are then blended with one or more non-solvent diluents to produce paste formulations at high solids content and high active ingredient concentration that are suitable for injection in low volumes into an animal (e.g., a human or a veterinary animal) in order to treat, ameliorate, prevent or diagnose a disease or physical disorder in the animal. The invention also provides such paste formulations produced by such methods of the invention.

In additional aspects, the present invention provides high concentration/high viscosity injectable formulations for parenteral (e.g., intracutaneous, subcutaneous and/or intramuscular) administration of therapeutic agents or active pharmaceutical ingredients, and methods of manufacturing such formulations in a way that results in the production of high-concentration/high-viscosity, storage-stable formulations, ready-to-use (i.e., that do not require reconstitution and/or dilution prior to use). For purposes of the present invention, the term “therapeutic agent” or “active pharmaceutical ingredient” or “pharmaceutically active ingredients” (these phrases are used interchangeably and equivalently herein, and are readily understood by those of ordinary skill in the art) encompasses drugs, vaccines, hormones (especially peptide hormones, e.g., insulin, glucagon, pramlintide, human growth hormone, prolactin, mammatrophic hormone, vasopressin, oxytocin, thyroxine, cortisol, and the like), antibodies (including monoclonal antibodies and polyclonal antibodies) or fragments thereof (e.g., Fab fragments, Fc fragments, etc.), antibody conjugates (comprising an antibody or a fragment thereof conjugated, i.e., directly or indirectly linked, to another active pharmaceutical ingredient), antibody complexes (e.g., multimeric immunoglobulin complexes), antibiotics, enzymes, and other biologics (e.g., growth factors, colony-stimulating factors, interleukins, interferons and the like), or small molecule active pharmaceutical ingredients (including but not limited to anti-cancer small molecule actives, antibiotics, anti-fungals, anti-inflammatories, anti-convulsives, anticoagulants and anti-thrombotics, anti-seizure therapeutics and preventatives, anti-migraine therapeutics and preventatives, and the like), that are used in the prevention, diagnosis, alleviation, treatment or cure of a condition, ailment or disease. In certain embodiments, the formulations comprise one or more polymer or copolymer carriers which provide for sustained release of the therapeutic compound, e.g., poly(ethylene glycol) (“PEG”), poly(lactic-co-glycolic acid) (“PLGA”), and the like. In certain such formulations, the therapeutic agent itself may be complexed or conjugated with one or more of such polymers or copolymers. In additional aspects, the formulations of the invention generally comprise one or more excipients, carriers or buffers, such as one or more sugars (e.g., trehalose, dextrose, sucrose, mannose, fructose and the like), one or more sugar alcohols (e.g., mannitol, xylitol, glycerol, erythritol, maltitol, sorbitol, and the like), one or more buffering agents (e.g., histidine, citrate, succinate, lactate and the like), one or more surfactants (e.g., span 20, polysorbate 20, polysorbate 80, Kolliphor® HS15), a triglyceride (e.g., Miglyol® 810, Miglyol® 812, Miglyol® 818, Miglyol® 829, Miglyol® 840), and the like), one or more amino acids (which may be any naturally occurring amino acid, e.g., histidine, proline, glycine, methionine, tryptophan, phenylalanine, arginine and cysteine and the like), and other pharmaceutically acceptable carriers, excipients and fillers that will be readily familiar to those of ordinary skill in the relevant arts.

Formulations provided by the invention are stable and typically do not require reconstitution prior to use, comprising from about 0.1 microliter up to about 3 mL of a concentrated semi-solid or solid formulation for a single-dose injectable formulation, and up to about 10 mL for an infusible formulation, comprising an effective amount of at least one therapeutic agent (and in some embodiments, more than one, e.g., two, three, four or more therapeutic agents in an admixture, particularly such coformulations in which two or more therapeutic agents are present that are not compatible with each other in a typical aqueous formulation) homogeneously contained within a pharmaceutically acceptable carrier. In certain such aspects, the formulations comprise from about 10% to about 95% solids, from about 15% to about 90% solids, or from about 20% to about 85% solids by weight, and in certain preferred embodiments from about 40% to about 70% by weight, particularly about 40%, about 42%, about 45%, about 50%, about 55%, about 60%, about 65%, about 67% or about 70%, by weight. In certain such aspects, the therapeutic or pharmaceutically active agent has a mean particle size range of from about 10 nanometers (0.01 micrometers) to about 100 micrometers, with no particles being larger than about 1 mm, and in certain such embodiments it has a mean particle size from about 0.1 micrometers to about 25 micrometers, with no particles being larger than about 25 micrometers, and in certain other embodiments it has a mean particle size of from about 1 to about 15 micrometers, particularly wherein at least about half of the particles range in size from about 2 micrometers to about 8 micrometers. In particular, the processes used to produce the present formulations result in formulations wherein the particles are relatively uniform in size, though not necessarily considered monodisperse. For example, the measured size distribution of the particles (e.g., measured by standard techniques such as laser diffraction and reported as the D₁₀, D₅₀ and D₉₀) can yield a span (typically defined in the field as ((D₉₀−D₁₀)/D₅₀)) ranging from 0.5-5.0, or 1.0-3.0, or 1.5-2.5. Ideally, the particles of the therapeutic agent are of a size and of a size distribution such as to promote high packing efficiency and minimal surface area, characteristics that can be controlled using the manufacturing processes provided by the invention as described elsewhere herein.

In certain embodiments, the formulation further comprises one or more carriers (e.g., one or more diluents, additives and/or polymers) which impart thixotropic properties to the formulation. The therapeutic agent is preferably homogeneously incorporated into the pharmaceutically acceptable carrier(s), and said formulation is in a thixotropic or non-Newtonian state in the form of a paste or slurry.

In certain preferred such embodiments, the therapeutic agent is present in powder form and is homogeneously contained within a pharmaceutically acceptable carrier. The carrier is preferably biocompatible and is a non-solvent to the therapeutic agent powder (such that no or minimal dissolution of the powder occurs in the carrier), and in certain preferred embodiments fills the spaces between the particles of the therapeutic agent powder in a way that makes them flow. In certain such embodiments, the carrier is selected from the group consisting of alkyl benzoates, aryl benzoates, aralkyl benzoates, triacetin, aprotic polar solvents (e.g., N-methyl-2-pyrrolidine 5 (NMP), dimethyl sulfoxide (DMSO)), medium chain triglycerides (MCTs, e.g., Miglyol® 810, Miglyol® 812 N, Miglyol® 818, Miglyol® 829, Miglyol® 840, and the like), alkanes, cyclic alkanes, chlorinated alkanes, fluorinated alkanes, perfluorinated alkanes and mixtures thereof. The carrier can be a single fluid or semi-solid, or a mixture of two or more fluids (or semi-solids) that may be either partially or fully miscible with each other, or that are immiscible with each other such as mixtures of two or more fluids that form an emulsion.

In certain embodiments, the injectable formulation may provide controlled (slow) or sustained release. In such embodiments, for example, the formulation may comprise a pharmaceutically acceptable polymer in an amount effective to slow the release of the therapeutic agent from said formulation upon administration via injection into the epidermal, dermal or subcutaneous layer of an animal. The agent(s) promoting controlled or sustained release of the active pharmaceutical (therapeutic) ingredient in such formulations may be incorporated into the continuous (diluent) phase and/or the dispersed (particulate matter) phase of the composition. Additionally, or alternatively, the therapeutic agent may be incorporated into liposomes or conjugated to or incorporated with polysaccharides and/or other polymers to provide a controlled release of the therapeutic agent from said formulation upon administration via injection into the epidermal, dermal or subcutaneous layer of an animal. In certain preferred embodiments, the therapeutic agent may be incorporated into a biocompatible polymer and a biocompatible solvent having low water miscibility that forms a viscous gel with the polymer and limits water uptake by the composition. Such compositions are disclosed, for example, in U.S. Pat. No. 6,130,200, which is incorporated by reference herein in its entirety, and for example utilize a PEG polymer or a PLGA copolymer together with an effective plasticizing amount of a solvent (e.g., comprising a lower alkyl or aralkyl ester of benzoic acid) to form a gel with the polymer.

In additional embodiments, the present invention also provides methods of administration of an injectable formulation into an animal (e.g., a human or a veterinary or agricultural animal) parenterally, e.g., intracutaneously (into the epidermis or dermis), subcutaneously or intramuscularly, to deliver higher concentrations or amounts of the active pharmaceutical ingredient into the animal in a lower volume than would be possible or effect pain-free or substantially pain-free administration of a therapeutic agent, comprising injecting from about 0.1 to about 50 microliters of an concentrated semi-solid or solid formulation (e.g., a slurry or paste) comprising from about 20 to about 85% solids, by weight and comprising an effective amount of a therapeutic agent into the epidermal, dermal or subcutaneous skin layer of an animal.

In preferred embodiments, the therapeutic agent is processed, e.g., via spray drying or lyophilization, to produce a particle size suitable for injection through a narrow gauge needle (e.g., 25 to 30 gauge). The therapeutic agent is typically processed into a powder alongside one or more excipients that are included, for example, to promote stability, achieve a desired pharmacokinetic profile and/or improve manufacturability of the therapeutic agent.

Exemplary processes for producing such formulations are provided elsewhere herein, particularly in the Examples hereinbelow.

In certain preferred embodiments, the therapeutic agent is incorporated into a nonaqueous or semi-aqueous pharmaceutically acceptable carrier. In further preferred embodiments, the formulation exhibits shear-thinning properties upon injection from an injection device.

The present invention is further directed in part to methods of treating animals, e.g., human patients or veterinary or agricultural animals, utilizing the injectable formulations, injection devices and methods of preparation of the present invention.

The term “intracutaneous” encompasses administration into the cutaneous, i.e., the epidermal or dermal, skin layer of an animal, e.g., a human or veterinary or agricultural animal.

The term “subcutaneous” means administration below the cutaneous skin layer but above the muscle layer of an animal, e.g., a human or veterinary or agricultural animal.

The term “intramuscular” means administration into the muscle layer of an animal, e.g., a human or veterinary or agricultural animal.

The term “pharmaceutically acceptable carrier” means a pharmaceutically acceptable solvent, suspending agent, diluent or vehicle for delivering a compound of the present invention to the animal or human. The carrier may be liquid, semi-solid or solid, and can be a Newtonian or Non-Newtonian fluid.

The term “pharmaceutically acceptable” ingredient, excipient or component is one that is suitable for use with humans and/or animals without (or with reduced) undue adverse side effects (such as toxicity, irritation, and allergic response) commensurate with a reasonable benefit/risk ratio.

The term “therapeutic agent” means an agent that effects a desired, beneficial, often pharmacological, effect upon administration to a human or an animal, whether alone or in combination with other pharmaceutical excipients or inert ingredients.

The term “chemical stability” means that with respect to the therapeutic agent, an acceptable percentage of degradation products produced by chemical pathways such as oxidation or hydrolysis is formed. In particular, a formulation is considered chemically stable if no more than up to about 50% , e.g., no more than about 10%, about 20%, about 30%, about 40% or about 50%, breakdown products are formed after one year of storage at the intended storage temperature of the product (e.g., room temperature); or storage of the product at 30° C./60% relative humidity for one year; or storage of the product at 40° C./75% relative humidity for one month, and preferably three to six months.

The term “physical stability” means that with respect to the therapeutic agent, an acceptable percentage of aggregates (e.g., dimers, trimers and larger forms) is formed. In particular, a formulation is considered physically stable if no more than about 15% aggregates are formed after one year of storage at the intended storage temperature of the product (e.g., refrigerated or room temperature); or storage of the product at 30° C./60% relative humidity for one year; or storage of the product at 40° C./75% relative humidity for one month, and preferably three to six months.

The term “stable formulation” means that at least about 65% chemically and physically stable therapeutic agent remains after two months of storage at room temperature. Particularly preferred formulations are those which retain at least about 80% chemically and physically stable therapeutic agent under these conditions. Especially preferred stable formulations are those which do not exhibit degradation after sterilizing irradiation (e.g., gamma, beta or electron beam).

The term “bioavailability” is defined for purposes of the present invention as the extent to which the therapeutic agent is absorbed from the formulation into the bloodstream and/or tissues of an animal or human to whom the formulation has been administered.

The term “systemic” means, with respect to delivery or administration of a beneficial agent to a subject, that beneficial agent is detectable at a biologically significant level in the blood plasma of the subject.

The term “pastes” means a concentrate of the therapeutic agent dispersed in a pharmaceutically acceptable carrier having a thick consistency to form a viscous injectable semi-solid. Pastes may be categorized as two-phase systems, with the particulate matter (i.e., solid phase) comprising the “dispersed phase” and the diluent (i.e. non-solvent) comprising the “continuous phase.”

The term “slurry” means a thin paste.

The terms “controlled-release” and “sustained release” are defined for purposes of the present invention as the release of the therapeutic agent at such a rate that blood (e.g., plasma) concentrations are maintained within the therapeutic range but below toxic concentrations over a period of time of about one hour or longer, preferably 12 hours or longer.

In certain aspects, the present invention provides syringes that have been pre-loaded with a high concentration/high viscosity pharmaceutical paste formulation of the invention. In certain such embodiments, pre-loaded syringes comprise a syringe body defining a reservoir, a paste disposed within the reservoir, the paste having a solids concentration of at least about, about, or greater than 100-1000 mg/mL (particularly about 100 mg/mL, about 200 mg/mL, about 300 mg/mL, about 350 mg/mL, about 400 mg/mL, about 425 mg/mL, about 450 mg/mL, about 475 mg/mL, about 500 mg/mL, about 525 mg/mL, about 550 mg/mL, about 575 mg/mL, about 600 mg/mL, about 625 mg/mL, about 650 mg/mL, about 675 mg/mL, about 700 mg/mL, about 750 mg/mL, about 800 mg/mL, about 850 mg/mL, about 900 mg/mL, about 950 mg/mL and about 1000 mg/mL, and most particularly about 300 mg/mL to about 850 mg/mL), a plunger and/or piston disposed within the reservoir and configured to be moved to dispense paste from the reservoir, a Luer fitting disposed on the syringe body and in fluid communication with the reservoir, and a sealing cap disposed on the Luer fitting to seal the reservoir. Some embodiments comprise a needle defining a lumen, the needle configured to be coupled to the syringe body via the Luer fitting to allow intracutaneous delivery of the paste, where the reservoir has an internal first transverse dimension larger than an internal second transverse dimension of the lumen. Embodiments of the present pre-loaded syringes may have the needle affixed to the syringe via a Luer-lock or Luer-slip (“slip-tip”) fitting. Alternative embodiments of the present invention may have the needle permanently affixed to the syringe body using, for example, a staked-needle configuration, wherein needle is not removable from the syringe body as with a Luer fitting.

In certain embodiments, the pre-loaded syringes comprise a syringe body defining a reservoir having an internal first transverse dimension, a paste disposed within the reservoir, the paste having a solids concentration of at least about, about, or greater than about 300-600 mg/mL, a needle defining a lumen having an internal second transverse dimension that is smaller than the first transverse dimension, the needle configured to be in fluid communication with the reservoir to allow intracutaneous delivery of the paste, and a plunger disposed within the reservoir and configured to be moved to dispense paste from the reservoir through the lumen.

In some embodiments of the present pre-loaded syringes, the paste has a volume of between 15, 40, 50, 100, 150, 250 or 500 μL and 1000, 2000, or 3000 μL. In certain aspects the paste can have a volume of between 15 μL and 1000 μL. In some embodiments, the paste has a volume of up to about 40 μL. In some embodiments, the paste has a volume of up to about 50 μL. In some embodiments, the paste has a volume of up to about 100 μL or about 150 μL. In some embodiments, the paste has a volume of up to about 200 μL to about 1000 μL, e.g., about 200 μL, about 300 μL, about 350 μL, about 400 μL, about 450 μL, about 500 μL, about 550 μL, about 600 μL, about 650 μL, about 700 μL about 750 μL, about 800 μL, about 850 μL, about 900 μL, about 950 μL or about 1000 μL.

Some embodiments of the present pre-loaded syringes are configured to dispense paste at a flow rate of at least about, about, or greater than 15 microliters per second (μL/s) under a force applied to the plunger having a magnitude of about or at most 50, 60, or 70 newtons (N). In certain aspect the force applied to the plunger can be below 5, 10, 20, 25, 30, 35, 40, 45, 50, 55, 60, 65, or 70 N. In a further aspect the force applied to the plunger can be below 25 N. Some embodiments are configured to dispense paste at a flow rate of greater than 65 μL/s under a force applied to the plunger having a magnitude of about or at most 50 to 70 N. In other aspects, particularly those using autoinjectors or other assisted delivery devices (e.g., reusable autoinjectors), the pre-loaded syringes or devices are configured to dispense paste at a flow rate of the above-described forces or even at higher forces than about 70 N, e.g., about 75 N, about 80 N, about 85 N, about 90 N, about 95 N or about 100 N.

Some embodiments of the present kits comprise a syringe body defining a reservoir having an internal first transverse dimension and a needle configured to be coupled to the syringe body and defining a lumen having an internal second transverse dimension that is smaller than the first transverse dimension. In some embodiments, the paste is disposed within the reservoir. In some embodiments, the syringe body comprises a Luer fitting (e.g. Luer-lock or Luer-slip fitting) in communication with the reservoir and a sealing cap disposed on the Luer fitting to seal the reservoir, where the needle is configured to be coupled to the syringe body via the Luer fitting. In other embodiments, the needle is integral to the syringe body without being detachably connected. In some embodiments, the reservoir has a volume of between 50, 75, or 100 μL and 1000, 2000, or 3000 μL.

Some embodiments of the present kits comprise a plunger disposed within the reservoir and configured to be moved to dispense paste from the reservoir through the lumen at a flow rate of greater than 30 μL/s under a force applied to the plunger having a magnitude such as those described elsewhere herein. Some embodiments comprise a plunger disposed within the reservoir and configured to be moved to dispense paste from the reservoir through the lumen at a flow rate of greater than 65 μL/s under a force applied to the plunger having a magnitude such as those described elsewhere herein.

An alternative embodiment is the use of bolus injectors, which are alternatively known as patch pumps or high-volume injectors. In certain aspects a patch pump can be employed for prolonged delivery of viscous pastes to a patient. Examples of these injectors include the SmartDose™ electronic wearable bolus injector (West Pharmaceutical Services, Inc.) and the Lapas® bolus injector (Bespak), and others that are known in the art (see, e.g., Badkar A. V. et al., Drug Des. Devel. Ther. 15: 159-170 (2021), doi:10.2147/DDDT. S287323). These devices can be worn on the body and can provide automated sub- or intra-cutaneous delivery of a high concentration paste at a slower infusion rate relative to a traditional auto-injector or manually operated syringe. In these devices the paste is filled in an internal reservoir and slowly infused into the patient at a low volumetric flow rate (relative to manual syringes and auto-injector devices). These devices may be worn like a patch adhered to the skin, delivering the medicament over the course of several minutes, or up to about an hour. As a non-limiting example of the volumetric flow rates that may be employed in these systems, delivery of 3 mL of a therapeutic paste over the course of 10 minutes would entail a delivery rate of 5 μL/second. Delivery of a 3 mL volume of paste over the course of 1 hour would entail a delivery rate of 0.83 μL/second.

Some embodiments of the present methods for intracutaneously injecting a volume of paste comprise moving a plunger of a syringe to dispense paste from a reservoir of the syringe through a lumen of a needle of the syringe, the reservoir having an internal first transverse dimension that is larger than an internal second transverse dimension of the lumen, e.g., where the second transverse dimension is between 0.1 and 0.9 mm, where the paste has a solids content of about 20% to about 80%, including all values and ranges therebetween, a solids concentration of greater than about 100 mg/mL, e.g., of about 300 to about 800 mg/mL including all values and ranges therebetween, and particularly an active pharmaceutical ingredient concentration of about 300 to about 600 mg/mL including all values and ranges therebetween, and where the paste is dispensed at a flow rate of greater than 30 μL/s as the plunger is moved at a rate of between 0.5 and 50 millimeters per second (mm/s). Some embodiments comprise disposing the needle into and/or through cutaneous tissue of a patient. Some embodiments comprise removing a sealing cap from a Luer fitting of the reservoir. Some embodiments comprise coupling the needle to the reservoir via a Luer fitting disposed on at least one of the needles and the reservoir. In some embodiments, the flow rate of the paste is substantially linearly proportional to the rate of plunger movement.

In some embodiments of the present methods, the injected volume of paste is greater than about 1 μL. In some embodiments, the injected volume of paste is between 15, 30, or 100 μL and 1200, 2000, or 3000 μL for single injection doses, and up to about 10 mL for infusion use. In some embodiments of the present syringes, kits, and/or methods, the first transverse dimension is larger than the second transverse dimension. In some embodiments, the first transverse dimension is between 1, 2, 3, 4 and 5, 6, 7, 8, 9, 10, 11, 12 mm, including all values and ranges there between. In some embodiments, the second transverse dimension is between 0.1, 0.2, 0.3, or 0.4 and 0.5, 0.6, 07, 0.8, or 0.9 mm, including all values and ranges there between.

In some embodiments of the present syringes, kits, and/or methods, the needle is a size of 18 Gauge or of higher gauge (where higher gauge represents a physically smaller needle in terms of needle external and/or internal diameter). In some embodiments, the needle has a size of 23 Gauge or smaller. In some embodiments, the needle has a size of 25 Gauge or 27 G or smaller (i.e., higher gauge). In some embodiments, the needle has an exposed length smaller than or about 50 mm. In some embodiments, the needle has an exposed length smaller than or about 40 mm. In some embodiments, the needle has an exposed length smaller than or about 13 mm. In some embodiments, the needle has an exposed length of approximately 8 mm. In some embodiments, the needle has an exposed length of approximately 6 mm.

In some embodiments of the present syringes, kits, and/or methods, the paste has a solids concentration of greater than 200 mg/mL. In some embodiments, the paste has a solids concentration of between 200 and 800 mg/mL. In some embodiments, the paste has a solids concentration of between 300 and 750 mg/mL. In some embodiments, the paste has a solids content of between 1% and 99%. In some embodiments, the paste has a solids content of between 30% and 75%. In some embodiments, the paste has a solids content of between 40% and 65% or between 50% and 60%. In some embodiments, the paste has a density of between about 0.5, 0.7, 0.75, 1.0, 1.1, 1.2, 1.3, to about 1.4, 1.5, 1.6, 1.7, 1.8, 1.9, 2.0 g/mL, including all values and ranges there between.

As used in this disclosure, a paste is a two-phase mixture of a solid (e.g., a powder containing a medicament and, if necessary, stabilizing agents and/or excipients) dispersed in a liquid (e.g., a biocompatible diluent), which is a non-solvent to, or which only minimally solubilizes, the solid (e.g., and thus, the diluent is typically, but not always, lipophilic in nature). A paste behaves as a solid until a sufficiently large load or stress is applied (typically referred to as the ‘yield stress’), at which point the paste flows like a liquid (e.g., pastes may be defined as semi-solids). Pastes may exhibit non-Newtonian fluid behavior, specifically shear-thinning flow characteristics and/or viscoelastic behavior.

The term “coupled” is defined as connected, although not necessarily directly, and not necessarily mechanically; two items that are “coupled” may be unitary with each other.

The terms “a” and “an” are defined as one or more unless this disclosure explicitly requires otherwise.

The term “substantially” is defined as largely but not necessarily wholly what is specified (and includes what is specified, e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, 10, and 20 percent.

As used herein, the term “intracutaneous injection” encompasses epidermal, intradermal, subcutaneous or intramuscular injection.

As used herein, a “phase” is defined as a homogeneous, physically distinct portion of a system that is separated from other portions of the system by bounding surfaces. It is known that there are three primary phases of matter (solid, liquid and gaseous). As an example, a system containing particulate matter suspended in a liquid that is a non-solvent to the particulate matter is considered a two-phase system. Conversely, a system consisting of organic macromolecules uniformly distributed throughout a liquid such that no apparent boundaries exist between the macromolecules and the liquid molecules is considered a single-phase solution.

As used herein, a “semi-solid” is an attribute of a material that exhibits plastic flow behavior. A semi-solid material is not pourable, does not readily conform to its container at room temperature, and does not flow at low shear stress. Accordingly, semi-solids have a yield stress that must be exceed before plastic (i.e., non-reversible) deformation occurs. Semi-solids typically have a viscoelastic rheological flow profile.

Accordingly, a semi-solid is not a specific physical composition or pharmaceutical dosage form, but rather refers to a physical property of the material. Thus, a variety of materials can be considered semi-solids, as they will possess the attribute of a semi-solid material, despite being physically distinct compositions. For example, the USP-NF describes both a cream and a medicated foam as having a semi-solid consistency, and thus both may be considered semi-solid fluids, or semi-solids, despite being otherwise physically distinct compositions. Similarly, gels and pastes are often both termed semi-solids, despite being physically distinct. Gels are defined by the USP-NF as a dosage form that is a semi-solid dispersion of small particles or a solution of large molecules interpenetrated by a solution containing a gelling agent to provide stiffness. Thus, gels may be either single-phase or two-phase systems. As defined in Remington: The Science and Practice of Pharmacy (2006), gel systems may be either clear or turbid, as the ingredients comprising the gel may not be completely soluble or insoluble, or they may form aggregates and disperse light. Gels are defined “as semi-rigid systems in which the movement of the dispersing medium is restricted by an interlacing three-dimensional network of particles or solvated macromolecules in the dispersed phase . . . the interlacing and consequential internal friction is responsible for increased viscosity and the semi-solid state.”

Gels in which the macromolecules are distributed throughout the liquid in such a manner that no apparent boundaries exist between them and the liquid are called single-phase gels. In instances in which the gel mass consists of floccules of small distinct particles, the gel is classified as a two-phase system and frequently called a magma or a milk. Gels and magmas are considered colloidal dispersions since they each contain particles of colloidal dimension. The generally accepted size range for a substance “colloidal” is when particles fall between 1 nm and 0.5 μm.

By contrast, pastes may be defined as a semi-solid dosage form containing a high percentage of finely dispersed solids with a stiff consistency. As discussed earlier, the actual solids content of the paste will primarily depend on the properties of the constituent powder. To prepare a paste, the minimum quantity of fluid that is added to a powder must be sufficient to coat and produce a monolayer of fluid around each individual powder particle. Note that this is an idealized situation where all powder-powder contacts have been fully disrupted, though in reality many micronized powders are highly cohesive, and complete disruption of all direct powder-powder contacts may not be possible, despite the application of high-shear mixing techniques. Additional fluid is then added to the mixture to fill in the interstitial spaces between the powder particles (i.e. the void volume) and thus enable the particles to flow as a fluid when the yield stress of the paste has been exceeded. Accordingly, powders possessing very low density (i.e. high surface area-to-volume ratio) and/or poor packing (i.e. larger interstitial spaces between particles) will require a greater volume/mass of fluid to form a paste compared to powders with a lower surface area-to-volume ratio and/or good packing. Thus, gels and pastes may both possess the semi-solid character, and may both be referred to as semi-solids, but they are physically distinct dosage forms. In particular, the solids concentration of a paste is typically much greater, and the particles are often much larger than the upper limit of the colloidal region (0.5 μm). Overall, the USP-NF defines at least six different dosage forms as being semi-solids, including creams, foams, gels, jellies, ointments, and pastes. However, it will be readily known and understood by the ordinarily skilled technician that these pharmaceutical dosage forms are distinct physical compositions having distinct physical properties, despite all having the semi-solid rheological attribute and thus being broadly termed semi-solids.

“Solids Content,” as used herein, refers to the percent-weight (%wt.) of solid phase (e.g., powder) in a paste as a fraction of the total mass of the two-phases that comprise the paste (solids phase plus liquid phase). Solids content is typically presented/discussed in units of %. As an example, if 1 g of paste was prepared by blending 0.65 g of solids phase with 0.35 g of liquid phase, the solids content of this paste is 65%.

“Solids Concentration,” as used herein, refers to the mass of the solids phase per unit volume of paste. Typical units for solids concentration include mg/mL and g/mL. Solids concentration of a paste is related to the solids content and can be obtained by multiplying the solids content of a paste by the density of the paste (measured using a suitable method such helium pycnometry). For example, the solids concentration of a paste having a density of 1250 mg/mL (1.25 g/mL) and a solids content of 60% would be approximately 750 mg/mL (0.75 g/mL). It is noted that the drug concentration in the solid phase of the paste would be no greater than the solids concentration of the paste, and typically is below the solids concentration due to the presence of additional components in the solid phase (e.g., bulking or stabilizing excipients) that dilute the drug concentration.

“Non-Newtonian,” as used herein, defines a fluid where the viscosity is dependent on the shear rate or shear rate history. This contrasts with a Newtonian fluid, where the viscosity is typically independent of the applied shear rate.

“Thixotropic,” as used herein, defines a fluid that exhibits a shear-thinning property. More specifically, a thixotropic fluid exhibits a time-dependent shear-thinning property, which contrasts with a pseudoplastic fluid, which may characterize a fluid that exhibits time-independent shear-thinning. However, for the purpose of this application, a thixotropic fluid describes shear-thinning fluids in general.

The term “pharmaceutically acceptable,” as used herein, means suited for normal pharmaceutical applications, i.e., giving rise to no serious adverse events in patients.

The term “pharmaceutically acceptable carrier” means a pharmaceutically acceptable solvent, suspending agent or vehicle for delivering a compound of the present invention to the animal or human. The carrier may be liquid, semi-solid or solid.

The term “pharmaceutically acceptable” ingredient, excipient or component is one that is suitable for use with humans and/or animals without undue adverse side effects (such as toxicity, irritation, and allergic response) commensurate with a reasonable benefit/risk ratio.

The term “therapeutic agent,” which is used interchangeably herein with the terms “pharmaceutically active ingredient”, “active ingredient” or “active pharmaceutical ingredient”, means an agent that effects a desired, beneficial, often pharmacological, effect upon administration to a human or an animal, whether alone or in combination with other pharmaceutical excipients or inert ingredients. In certain aspects of the present invention a therapeutic agent encompasses drugs (e.g., small molecules, peptides, proteins, biologics), vaccines, oligonucleotides, gene therapy vehicles/vectors, and the like used in the prevention, diagnosis, alleviation, treatment or cure of a condition, ailment or disease.

The term “chemical stability” means that with respect to the therapeutic agent, an acceptable percentage of degradation products produced by chemical pathways such as oxidation or hydrolysis is formed. In particular, a formulation is considered chemically stable if no more than about 20% breakdown products are formed after one year of storage at the intended storage temperature of the product (e.g., 4° C. (refrigerated), or 25° C. (room temperature)); or storage of the product at 30° C./60% relative humidity for one year; or storage of the product at 40° C./75% relative humidity for one month, and preferably three to six months.

The term “physical stability” means that with respect to the therapeutic agent, an acceptable percentage of aggregates (e.g., dimers, trimers and larger forms) is formed. In particular, a formulation is considered physically stable if no more than about 15%, and preferably no more than about 1-10% or about 1-5%, aggregates are formed after one year of storage at the intended storage temperature of the product (e.g., room temperature); or storage of the product at 30° C./60% relative humidity for one year; or storage of the product at 40° C./75% relative humidity for one month, and preferably three to six months.

The term “stable formulation” means that at least about 65% chemically and physically stable therapeutic agent remains after two months of storage at room temperature. Particularly preferred formulations are those which retain at least about 80% chemically and physically stable therapeutic agent under these conditions.

The term “bioavailability” is defined for purposes of the present invention as the extent to which the therapeutic agent is absorbed from the formulation.

The term “systemic” means, with respect to delivery or administration of a beneficial agent to a subject, that beneficial agent is detectable at a biologically significant level in the blood plasma of the subject.

The term “slurry” means a thin paste (the term “paste” is defined hereinbelow).

The term “controlled-release” is defined for purposes of the present invention as the release of the therapeutic agent at such a rate that blood (e.g., plasma) concentrations are maintained within the therapeutic range but below toxic concentrations over a period of time of about one hour or longer, preferably 12 hours or longer.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, an apparatus that “comprises,” “has,” “includes,” or “contains” one or more elements possesses those one or more elements, but is not limited to possessing only those elements. Likewise, a method that “comprises, ” “has,” “includes,” or “contains” one or more steps possesses those one or more steps, but is not limited to possessing only those one or more steps.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Other objects, advantages, and features of the present invention will be readily apparent to those of ordinary skill in the art upon review of the description, drawings, examples and claims presented herein.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 is a pair of scanning electron photomicrographs showing particles of therapeutic protein powders (in this case, monoclonal antibodies (mAbs)) prepared according to the spray-drying methods of the invention using different spray-dryer (Buchi B290) apparatus settings. FIG. 1A: inlet temperature 90° C., aspirator 65% (27 m³/hr), nozzle gas rate 473 L/hour (pressure drop 0.41 bar), feed solution pump rate 3% (about 1 mL/min), and no secondary drying or processing (e.g., sieving) after spray-drying. FIG. 1B: inlet temperature 70° C., aspirator 85% (34 m³/hr), nozzle gas rate 473 L/hour (pressure drop 0.41 bar), feed solution pump rate 10% (about 3 mL/min), and secondary drying (lyophilization) and sieving were performed after spray-drying.

FIG. 2 is a series of scanning electron photomicrographs showing particles of therapeutic protein powders (in this case, monoclonal antibodies) prepared according to the spray-drying methods of the invention using different spray-dryer apparatus settings, as set forth in Table 4 hereinbelow. FIG. 2A: Formulation 1; FIG. 2B: Formulation 2; FIG. 2C: Formulation 3; FIG. 2D: Formulation 4; FIG. 2E: Formulation 5; FIG. 2F: Formulation 6; FIG. 2G: Formulation 7; FIG. 2H: Formulation 8.

FIG. 3 is series of bar graphs showing the size distribution (assessed by visual examination via scanning electron microscopy) of particles of therapeutic protein powders (in this case, monoclonal antibodies) prepared according to the spray-drying methods of the invention using different spray-dryer apparatus process settings. Formulation numbers and spray-dryer settings correspond to those set forth in the description of FIG. 2, above. FIG. 3A: Formulation 1; FIG. 3B: Formulation 2; FIG. 3C: Formulation 3; FIG. 3D: Formulation 4; FIG. 3E: Formulation 5; FIG. 3F: Formulation 6; FIG. 3G: Formulation 7; FIG. 3H: Formulation 8.

FIG. 4 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 5 is a chart (top) showing the percent aggregation of certain formulations prepared according to the methods of the invention prior to and after spray-drying (at time zero after spray-drying and after one day storage at 50° C. after spray-drying), and a bar graph depicting these results graphically. Secondary drying was performed after spray drying under reduced pressured in a lyophilizer (lyo).

FIG. 6 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 7 is a chart showing the percent aggregation of certain formulations prepared according to the methods of the invention prior to and after spray-drying (at time zero (T=0) after spray-drying and after five days storage at 40° C. post-drying).

FIG. 8 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 9 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 10 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 11 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 12 is a chart (top) showing the percent aggregation of certain formulations prepared according to the methods of the invention prior to and after spray-drying (at time zero after spray-drying and after one day storage at 50° C. after spray-drying), and a bar graph depicting these results graphically.

FIG. 13 is set of charts showing the main peak (FIG. 13A), acidic variants (FIG. 13B) and basic variants (FIG. 13C) observed upon ion exchange chromatography of cysteine-containing formulations prepared according to the methods of the invention prior to (“pre-SD”) and after spray-drying (at time zero (t0) after spray-drying and after one day storage at 50° C. after spray-drying (50C×1d)).

FIG. 14 is a comparison of representative tracings of two formulations, one containing 1.5 mg/mL cysteine (FIG. 14A) and the other containing 6 mg/mL cysteine (FIG. 14B) prepared according to the methods of the invention prior to (“pre-SD”) and after spray-drying (at time zero (“t0”) after spray-drying and after one day storage at 50° C. (“50C×1d”) after spray-drying).

FIG. 15 is a chart (top) showing the percent aggregation of certain formulations prepared according to the methods of the invention prior to and after spray-drying at different inlet temperatures (at time zero after spray-drying and after one day storage at 50° C. after spray-drying), and a bar graph depicting these results graphically.

FIG. 16 is a series of scanning electron photomicrographs showing particles of therapeutic peptide powders (in this case, monoclonal antibodies) spray-dried from the commercial formulations, showing particles observed in spray-dried formulations of Herceptin (TmAb) (FIG. 16A), Erbitux (cetuximab) (FIG. 16B) and Privigen (immune globulin) (FIG. 16C).

FIG. 17 is a chart showing the particle size distribution in the three formulations depicted in FIG. 16, as measured using laser diffraction. D₁₀: tenth percentile particle size; D₅₀: fiftieth percentile particle size; D₉₀: ninetieth percentile particle size. Span=(D₉₀−D₁₀/D₅₀).

FIG. 18 is a line graph depicting the injection force required (measured using a texture analyzer) to dispense 1 mL of paste formulations of the invention using a 1 mL long syringe with a 23 G needle (bottom tracings) or a 27 G needle (top tracings).

FIG. 19 is an ion exchange chromatogram showing the peaks obtained in formulations of trastuzumab (Herceptin®) in the commercial aqueous form (red tracing), a powder prepared by the spray-drying methods of the invention and then reconstituted in water (green tracing), or a XeriJect™ paste formulation of the invention (pink tracing).

FIG. 20 is a line graph (top) showing the pharmacokinetics (PK) of formulations of trastuzumab (Herceptin®) injected into test animals and assessed for plasma antibody concentration in the commercial aqueous form injected intravenously (blue tracing), and two XeriJect™ paste formulations of the invention injected subcutaneously into test animals, using a dose of 10 mg/Kg and the other 20 mg/Kg (FIG. 20A); and a chart showing certain PK parameters in tabular form (FIG. 20B).

FIG. 21 is a pair of scanning electron photomicrographs of a human enzyme formulation powder prepared from a commercial aqueous formulation by lyophilization (FIG. 21A) or by the spray-drying methods of the present invention (FIG. 21B).

FIG. 22 is a series of line graphs showing pharmacokinetic (PK) results of intravenous injection (FIG. 22A) of a commercial aqueous formulation of the enzyme used in FIG. 21, or of subcutaneous injection of the aqueous enzyme formulation (FIG. 22B, “Group 2”)) or a Xeriject™ paste of the enzyme prepared according to the methods of the present invention (FIG. 22B, “Group 3”).

FIG. 23 is a series of line graphs showing pharmacodynamic results of intravenous injection (FIG. 23A) of a commercial aqueous formulation of the enzyme used in FIG. 21, or of subcutaneous injection of the aqueous enzyme formulation (FIG. 23B, “Group 2”)) or a Xeriject™ paste of the enzyme prepared according to the methods of the present invention (FIG. 23B, “Group 3”).

FIG. 24 is series of line graphs showing pharmacokinetic (FIG. 24A) and pharmacodynamic (FIG. 24B) results of subcutaneous injection of a commercially available aqueous glucagon formulation (“GEK” in FIGS. 24A and 24B) or a Xeriject™ paste of glucagon prepared according to the methods of the present invention (“Xeris Paste” in FIGS. 24A and 24B).

FIG. 25 is a pair of scanning electron photomicrographs of a human recombinant protein formulation powder prepared from a commercial aqueous formulation by the spray-drying methods of the present invention. FIG. 25A: low-concentration feed solution; FIG. 25B: high-concentration feed solution.

FIG. 26 is a bar graph depicting the injection force required (measured using a texture analyzer) to inject about 150 μL of a recombinant protein paste prepared from the powders shown in FIG. 25, using commercially available large and small syringes affixed with either regular wall or thin wall 27 G needles, at a volumetric flow rate of 30 μL per second.

FIG. 27 is a pair of scanning electron photomicrographs of a human monoclonal antibody (bevacizumab, BmAb) formulation powder prepared by the spray-drying methods of the present invention. FIG. 27A: formulation XJ-1 (pH 4.0); FIG. 27B: Formulation XJ-2 (pH 6.0).

FIG. 28 is a pair of pharmacokinetic line graphs showing plasma concentrations over time of various formulations of XeriJect bevacizumab (BmAb) after injection into minipigs. FIG. 28A: linear scale; FIG. 28B: same results, but on a semilogarithmic scale.

FIG. 29 is a bar graph showing the time to maximum plasma concentration (Tₘₐₓ) for various formulations of XeriJect bevacizumab after injection into minipigs.

FIG. 30 is a pair of bar graphs showing the maximum plasma concentration (Cₘₐₓ) for various formulations of XeriJect bevacizumab after injection into minipigs, either uncorrected (FIG. 30A) or corrected for dose (FIG. 30B).

FIG. 31 is a bar graph showing the plasma half-life (T_{1/2}) for various formulations of XeriJect bevacizumab after injection into minipigs.

FIG. 32 is a pair of bar graphs showing dose-corrected total animal exposure for various formulations of XeriJect bevacizumab after injection into minipigs. FIG. 32A: dose-corrected AUCₗₐₛₜ; FIG. 32B: dose-corrected AUC_{∞}.

FIG. 33 is a bar graph showing the dose-corrected partial animal exposure, 14 days post-injection (AUC₃₃₆), for various formulations of bevacizumab after injection into minipigs.

FIG. 34 is a line graph showing the mean (±SEM) plasma insulin concentration after subcutaneous administration of Humulin R and XeriJect insulin formulations in Yucatan minipigs.

FIG. 35 is a line graph showing the mean (±SEM) blood glucose concentration after subcutaneous administration of Humulin R and XeriJect insulin formulations in Yucatan minipigs.

FIG. 36 is a pair of scanning electron photomicrographs of an exemplary spray dried IgG powder formulation produced by the methods of the present invention. Images are provided at different magnifications. Two different magnifications are depicted, at higher (FIG. 36A; scale bar=10 μm) and lower (FIG. 36B; scale bar=20 μm) magnification.

FIG. 37 is a line graph showing the particle size distribution analysis of an exemplary spray dried IgG powder formulation produced by the methods of the present invention.

FIG. 38 is a series of scanning electron photomicrographs of an exemplary spray dried IgG paste produced by the methods of the present invention. Images are provided at different magnifications. Three different magnifications are depicted, at higher (FIG. 38A; scale bar=10 μm and 38B, scale bar=8 μm) and lower (FIG. 38C; scale bar=20 μm) magnification.

FIG. 39 is a line graph showing the particle size distribution analysis of an exemplary spray dried IgG powder formulation produced by the methods of the present invention. X axis: distance (mm); Y axis: force (N).

### DETAILED DESCRIPTION OF THE INVENTION

Unless defined otherwise, all technical and scientific terms used herein have the same meanings as commonly understood by one of ordinary skill in the arts to which this invention belongs. Although any methods and materials similar or equivalent to those described herein can be used in the practice or testing of the present invention, the preferred methods and materials are described hereinafter.

Prior work by some of the present inventors has described methods for producing formulations prepared in the form of high solids concentration pastes capable of containing relatively high concentrations of pharmaceutically active compounds (see, e.g., U.S. Pat. Nos. 8,790,679, 8,110,209, and 9,314,424, and U.S. Patent Publication Nos. US 2017/0007675 and US 2017/0216529, the disclosures of all of which are incorporated herein by reference in their entireties). The present invention represents a dramatic expansion and improvement of this earlier work, resulting in formulations that not only have higher solids content and that are thus capable of containing higher concentrations of active ingredients (resulting in lower injection volumes and the ability to more readily control the pharmacokinetics and pharmacodynamics of the active substances upon delivery to a patient, particularly when injected subcutaneously into a patient) but that may also be storage stable for extended periods of time. Moreover, using the methods provided by the present invention, formulations can be produced comprising active ingredients of higher molecular weight and/or of higher solids content than had previously been thought possible and then had been previously demonstrated.

There are at least four elements to this invention that produce the effect of administration of a concentrated therapeutic agent into the intracutaneous space of a patient resulting in systemic circulation of the agent or exposure of an antigen to the immune system in the case of vaccines or targeted therapeutic products. These elements are as follows: (i) a low volume injection, relative to a higher volume injection that is needed when using a corresponding (typically aqueous) solution prepared at a significantly lower therapeutic agent concentration; (ii) a concentrated therapeutic agent (e.g., drug) particle group or dispersed solid formulation surrounded by a protective solution (typically non-aqueous); (iii) narrow diameter needle suitable for intracutaneous administration; and (iv) a shallow injection of the concentrated dispersion of therapeutic agent (e.g., paste, slurry) into the epidermal, dermal, subcutaneous or intramuscular layer of the skin. Compositions meeting all of these elements are provided by the manufacturing methods of the present invention which provide enhancements in both the solids loading capable of being achieved, as well as storage stability of the resulting paste formulations, when compared to prior composition and methods.

### Compositions and Methods of Manufacturing Same

In a first aspect, the present invention provides methods of producing or manufacturing high solids content and viscous pastes capable of being delivered through typical needles used for intracutaneous and/or intramuscular injection and comprising one or more active pharmaceutical ingredients, and compositions produced by such methods. In certain such aspects, the invention provides methods whereby aqueous formulations are dried into powder formulations and used to prepare high solids concentration paste formulations when mixed with a non-aqueous diluent that may contain relatively high active ingredient concentration and which are suitable for administration in relatively low volumes into an animal (e.g., a human or veterinary animal) to deliver a therapeutic bolus of one or more active pharmaceutical ingredients in a way that minimizes the discomfort and/or injection reaction experienced by the animal post-injection. Prior such methods utilized certain lyophilization, spray-drying, and other particle engineering approaches to prepare the powders used in making the paste formulations, but the present inventors have found that such methods resulted in pastes that had an undesirable upper limit of solids content and active ingredient concentration. Thus, one objective of the present invention was to identify suitable manufacturing methods that would allow the formation of paste formulations containing higher solids concentration and increased active content that can be delivered intracutaneously and/or intramuscularly in relatively low volumes using commercially available syringe/needle combinations, without resulting in the clogging of the needles or the need for use of excessive injection forces and/or delivery times which often causes discomfort to the animal receiving the therapeutic.

Thus, in one aspect the present invention provides methods of preparing paste formulations suitable for use according to the objectives of the invention. The present inventors have discovered that the preparation of high solids concentration pastes requires the starting powder material, containing both the active pharmaceutical ingredient(s) and one or more pharmaceutically acceptable excipients or carriers, to be produced in a way that results in the formation of powder particles that are preferably spherical in shape and having a preferred size and size distribution (as characterized using standard techniques such as laser diffraction). As described in the Examples herein, such powders can advantageously form higher solids concentration pastes (with the potential for a high therapeutic agent content) than have been previously available.

Suitable production methods for making such powders are described in detail in

Examples 1 and 2 hereinbelow. Briefly, aqueous formulations of one or more active pharmaceutical ingredients are buffer exchanged (e.g., 4-25° C.) against a suitable solution to promote protein concentration and/or buffer exchange. Such suitable solutions may comprise one or more saccharides (e.g., trehalose or sucrose), one or more buffering and/or stabilizing agents (e.g., lactate, citrate, succinate, histidine, phosphate, glycine, arginine, proline, methionine, etc.), one or more surfactants/surface active agents (e.g., polysorbate 20, polysorbate 80), and the like. Passive and/or active methods to promote protein concentration and/or buffer exchange can be used, including, but not limited to, dialysis, filtration, and centrifugation. For example, aqueous formulations may also be prepared using tangential flow filtration (TFF) at room temperature, followed by addition of one or more saccharides (e.g., trehalose or sucrose), one or more buffering and/or stabilizing agents (e.g., lactate, citrate, succinate, glycine, proline, histidine, arginine, methionine, etc.), one or more surface active agents (e.g., polysorbate 20, polysorbate 80), and the like, directly to the solution. Following concentration and/or buffer exchange using a suitable method (e.g., dialysis, TFF, etc.) the aqueous formulation is subjected to two stages of drying: (1) spray-drying a powder using a fixed set of settings on the spray drying instrument (e.g., BUCHI B-290 mini-spray dryer)—i.e., an inlet temperature of between about 70° C. and about 90° C., and preferably about 70° C.-80° C.; a drying gas flow rate of about 40-60 mm as measured by the B-290 ball flow meter (corresponding to approximately 470-800 L/hr); an aspirator flow rate of between 70%-100% according to the B-290 controls (corresponding to approximately 30-40 m³/hr), and preferably about 85%, 90%, 95% or 100%; and a feed flow rate of about 3-20% according to the B-290 controls (corresponding to 1-6 mL/min); and (2) drying the spray dried powder under reduced pressure (vacuum) for a suitable period of time to reduce the moisture content of the powder to a specified level (e.g., less than 5% (w/w), less than 4% (w/w), less than 3% (w/w), less than 2% (w/w) or less than 1% (w/w)). The subsequent drying step after spray drying may be referred to as a secondary drying step and can be performed using various techniques known in the art (e.g., under reduced pressure at ambient or other temperatures, under a continuous stream of inert gas at ambient or others temperatures, etc.).

Following production, the resultant powders are processed (e.g., sieved, milled, etc.) if desired to reduce the agglomerates present in the bulk solids phase. Using this approach, the present inventors have been successful at preparing powder starting materials that appear to have a more uniform spherical morphology and of generally polydisperse particle size distributions (e.g., where polydisperse indicates the measured particle size span (D₉₀−D₁₀/D₅₀)≠1.00), providing more suitable starting materials for preparation of the high solids concentration paste formulations of the invention as discussed in the examples below.

A paste can be generally described as a two-phase composition, where a solid phase (e.g., particulate matter, powder) is blended with a liquid phase (e.g., diluent), and wherein the solid phase is generally insoluble, or at least not fully soluble, in the liquid phase. In such a mixture the liquid phase may be referred to as the continuous phase and the solid phase is referred to as the dispersed phase. As is understood by the PHOSITA, the terms ‘continuous phase’ and ‘dispersed phase’ may also be used to describe compositions prepared from mixtures having the same phase, such as from two or more liquids that are not fully miscible with each other, non-limiting examples of which include oil-in-water (O/W) and water-in-oil-in-water (W/O/W) emulsions. A two-phase composition is distinct from a single-phase composition such as a solution, which is generally described as a preparation that contains one or more dissolved chemical substances in a suitable solvent or mixture of mutually miscible solvents.

In general, a paste can be defined as a multi-component formulation residing on the spectrum between a solution and a wetted solid. In a solution the solids concentration is sufficiently low that the particles will eventually begin to settle (e.g., under gravity) when the vial has been left undisturbed over a storage period relevant to a commercial drug product (e.g., 1 month, 6 months, 12 months, 18 months, 24 months). On the opposite end of the spectrum is a wetted solid, where there is an excess of solids phase relative to the liquid phase, resulting in a texture that can be broadly described as a wetted sand, loam, silt and/or clay. In contrast to a paste, a wetted solid would not readily flow through a syringe-needle combination suitable for intracutaneous and/or intramuscular injection and can be prone to breaking up and/or crumbling under applied shear (for example, using an oscillating and/or rotational rheometer), while a paste would be flowable and generally spread evenly under similar shear conditions.

To prepare a paste, a quantity of a non-solvent fluid (diluent) is added to a powder sufficient to produce at least a coating of fluid around the powder particles. Note that this is an idealized situation where all direct powder-powder contacts between individual powder particles have been fully disrupted, though in practice many micronized powders are highly cohesive (including in the particle size range described herein) and complete disruption of all direct powder-powder contacts may not be possible, despite the application of powder processing (e.g., milling, sieving, grinding, etc.) and/or high-shear mixing techniques. Additional fluid may then be added to the mixture to fill in the interstitial spaces between the powder particles (i.e., the void volume) and thus enable the particles to flow as a fluid when the yield stress of the paste has been exceeded. Accordingly, powders possessing very low density (i.e., high surface area-to-volume ratio) will require a greater amount of fluid to form a paste compared to powders with a lower surface area-to-volume ratio. On the spectrum between suspension and wetted solid described above, a paste can be formed across a range of solids content (e.g., 48-55%) where within that range the consistency of the paste may differ (e.g., increasing stiffness at the higher solids content region relative to the lower solids content region of the range) but the composition remains distinct from a suspension or wetted solids (e.g., with respect to its flow properties and/or resistance to solid phase settling over a relevant storage period). The solids content region/range where a paste is observed/formed will depend on the properties of its constituent liquid and solid phases, though the solid phase will generally have the greatest influence. By way of non-limiting example, as described above, powders having lower densities and/or larger specific surface areas (e.g., as measured via nitrogen adsorption) may form a paste in a lower solids content range (e.g., 15-23%) relative to a powder having a higher density and/or smaller specific surface area that may form a paste in a higher solids content range (e.g., 58-64%). Despite the large difference in solids content, both compositions would be distinct from suspensions or wetted solids, as described previously.

Pastes are typically referred to as semi-solid and/or viscoelastic compositions, which are broad terms intended to describe the rheological properties/behavior of a composition/substance. These terms (‘semi-solid’ and/or ‘viscoelastic’) can encompass a broad range of pharmaceutical dosage forms. Thus, gels and pastes may both possess a semi-solid character, and may both be referred to as semi-solids, but they are physically distinct dosage forms. Particularly, the solids concentration of a paste is typically much greater and the particles are generally much larger than the upper limit of the colloidal region (0.5 μm). Overall, the USP-NF defines at least six different dosage forms as being semi-solids, including creams, foams, gels, jellies, ointments, and pastes. However, it will be readily known and understood by the skilled technician that these pharmaceutical dosage forms are distinct physical compositions, despite all having the semi-solid attribute and thus broadly termed semi-solids.

Pastes can be formed from the powders provided by the methods of the present invention by blending the powder with one or more fluid non-solvent materials. Non-solvent fluids suitably used in preparing pastes according to this aspect of the invention include oils such as Miglyol 810 or 812, or other pharmaceutically acceptable compounds, non-limiting examples of which include triacetin or benzyl benzoate. Preferable for use according to the methods of the present invention are triacetin and/or Miglyol 812, which are mixed/blended with a powder containing one or more active pharmaceutical ingredients to produce a paste formulation to result in a paste having a solids concentration of at least about 30%, preferably at least about 40%, preferably at least about 50%, at least about 55%, at least about 60%, at least about 65%, or at least about 70%. Such pastes can then be loaded into commercially available syringes (either wide-bore or small-bore) affixed with commercially available needles (e.g., 23 G, 25 G, 27 G or 30 G) of appropriate gauge, wall thickness and length for the intended route of administration, and used to deliver the therapeutic pastes in relatively low volumes via intracutaneous and/or intramuscular injection into an animal.

As is known in the art (particularly as described elsewhere herein), it may be shown that injection force and/or flow resistance for a given syringe, needle, and/or substance combination can be substantially dominated by viscous effects near a reservoir exit, for example, due to the sharp change in cross-sectional area proximate this region as the wider barrel of the syringe leads to the much smaller lumen of the needle. Further, it has been observed that in pastes containing cohesive, micronized powders that are highly susceptible to forming robust agglomerates (where an agglomerate is comprised of two or more powder particles that have not been completely dispersed during processing, mixing and/or that may form over long-term storage) may exhibit partial, and/or complete clogging during delivery of the paste from the syringe reservoir and into the needle. Complete clogging results in the total obstruction of fluid flow from the device. In contrast, partial clogging does not result in the complete obstruction of fluid flow but may be noted as an abrupt increase in force/pressure during delivery and potentially resulting in a discontinuity during fluid delivery.

The present invention thus addresses these limitations by providing methods of producing both starting materials and formulations that ultimately result in a freer flow of the pastes from the syringes, without complete clogging and without resulting in particle agglomeration in the formulations.

In certain embodiments, the paste formulation is disposed within the syringe reservoir, which may be made of any material that is suitable for the intended application and that is compatible with the paste formulation. Non-limiting examples of reservoir materials include glass (e.g. borosilicate glass) and plastics (e.g. polypropylene, polycarbonate, polystyrene, cyclic olefin polymers and copolymers, etc.). As described above, the reservoir can comprise any suitable dimensions, and any suitable volume of the reservoir may comprise the paste. For example, in some embodiments, the paste has a volume of between 15 μL and 1000 μL. In some embodiments, the paste can have a volume greater than 50 μL, and in some embodiments, the paste can have a volume greater than 100 μL. In some embodiments, the paste can have a volume greater than 1000 μL, and in some embodiments, the paste can have a volume greater than 2000 μL. A volume of paste disposed within the reservoir may sometimes be referred to as an injection volume (e.g., if substantially all of the volume of paste is to be injected and/or dispensed from the syringe).

In certain embodiments, syringes with needles integral thereto (i.e., attached as a permanent fixture to the syringe without being removably attached such as via a Luer lock) can be suitably used to deliver the formulations of the present invention. Such syringe/needle combinations will suitably consider the dimension requirements of both the syringe and the needle noted hereinabove. Syringe/needle combinations useful in accordance with such aspects of the invention are available commercially, e.g., from Becton Dickinson or Medtronic/Covidien.

Pastes suitable for use in accordance with the present invention can comprise any suitable material properties (e.g., solids concentrations, solids content, viscosity profile, density, and/or the like). For example, in some embodiments the paste can comprise a solids concentration of greater than 100 mg/mL, greater than 200 mg/mL, or between 300 and 500 mg/mL (e.g., greater than any one of or between any two of 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 900, 950, 1000 or greater mg/mL). By way of further example, the paste can comprise a solids content (e.g., a mass of powder relative to a total mass of the paste) of between about 30% and about 70% (e.g., greater than any one of, or between any two of 1, 5, 10, 15, 20, 25, 30, 35, 40, 45, 50, 55, 60, 65, 70, 75, 80, 85, or more %, and suitably about 30%, about 35%, about 40%, about 45%, about 50%, about 55%, about 60%, about 65% or about 70%). By way of yet further example, the paste can comprise a density of between 1.0 and 1.4 g/mL (e.g., 1.20 g/mL) (e.g., greater than any one of or between any two of 1.0, 1.1, 1.2, 1.3, 1.4, 1.5, 1.6, 1.7, 1.8, 1.9, 2, or larger g/mL). With respect to the active pharmaceutical ingredient (“API”) component(s) of the pastes, the pastes of the present invention can comprise an API content relative to total mass of the paste of at least about 20%, e.g., from about 20% to about 70% API, from about 30% to about 65% API, about 35% to about 60% API, from about 20% to about 50% API, from about 25% to about 50% API, from about 20% to about 40% API, from about 30% to about 40% API, or from about 30% to about 35% API, including all values within these ranges.

In certain aspects a suitable paste may be a protein paste having a solids content of 35%, a density of 1.15 g/mL, and a solids concentration of approximately 400 mg/mL. In certain other aspects a suitable paste may be a protein paste having a solids content of 50%, a density of 1.25 g/mL, and a solids concentration of approximately 625 mg/mL. In certain other aspects a suitable paste may be a protein paste having a solids content of 65%, a density of 1.3 g/mL, and a solids concentration of approximately 845 mg/mL. In one example, such a paste can be tested, characterized, or optimized by dispensing the paste from a variety of syringes, each equipped with a 21, 22, 23, 25 or 27 Gauge regular wall, thin-wall, ultra-thin wall, extra-thin wall, special-thin wall needle, or the like, with the needles having differing exposed lengths (e.g., 0.25, 0.5, 1.0 inches) to determine the optimum commercially available needle and syringe combination for delivering a particular paste in a low volume while minimizing the injection force needed to deliver the paste to the animal subcutaneously.

Volumetric flow rate (e.g., microliters/second (μL/s)) can depend on the cross-sectional dimensions of the reservoir and plunger velocity. Thus, volumetric flow rates between syringes with differing reservoirs may be matched by varying applied plunger velocities amongst the syringes. For example, syringes having reservoirs with smaller internal transverse dimensions (e.g., 100 μL volume reservoirs) can require higher plunger velocities than syringes having reservoirs with larger internal transverse dimensions (e.g., 1000 μL volume reservoirs) to attain a given volumetric flow rate. For further example, for four illustrative syringes having reservoirs of varying volumes and internal dimensions, TABLE 1 provides respective plunger velocities required to achieve two particular volumetric flow rates: 33.3 μL/s and 67.0 μL/s.

TABLE 1
Plunger Velocities at Two Illustrative Flow Rates
for Reservoirs Consistent with Certain Syringes

| | 33.3 μL/s | 67.0 μL/s |
|---|---|---|
| Reservoir Volume | Plunger Velocity | Plunger Velocity |
| (μL) | (mm/s) | (mm/s) |
| 100 | 19.91 | 40.00 |
| 250 | 8.02 | 16.12 |
| 500 | 3.90 | 7.83 |
| 1000 | 2.00 | 4.01 |

As shown, in some embodiments the syringes are configured to dispense paste at a flow rate of greater than 30 μL/s as the plunger is moved at a rate of between 2 and 40 mm/s. Also, as shown in the depicted examples, flow rate of paste is substantially linearly proportional to the rate of plunger movement.

Some embodiments of the present methods for intracutaneously injecting a volume of paste comprise moving a plunger of a syringe to dispense paste from a reservoir of the syringe through a lumen of a needle of the syringe, the reservoir having an internal first transverse dimension that is larger than an internal second transverse dimension of the lumen, where the second transverse dimension is between 0.1 and 0.9 mm, where the paste has a solids concentration of greater than 100 mg/L, and where the paste is dispensed at a flow rate of greater than 30 μL/s as the plunger is moved at a rate of between 2 and 40 mm/s. Some methods comprise removing a sealing cap from a fitting (e.g., a Luer fitting) of the reservoir. Some methods comprise coupling the needle to the reservoir via a Luer fitting disposed on at least one of the needle and the reservoir. Some methods comprise disposing the needle into and/or through cutaneous tissue of a patient.

In some methods, the injected volume of paste is greater than 10 μL. In some methods, the injected volume of paste is between 15, 500, or 1000 μL to 1200, 2000, or 3000 μL. In some methods, the injected volume of paste is between 30 μL and 100 μL.

### Pharmaceutically Active Ingredients

The compositions of the present invention suitably comprise one or more (e.g., 1, 2, 3, 4, 5 or more) pharmaceutically active ingredients (used interchangeably herein with “active pharmaceutical ingredients” or “therapeutic ingredients”). By a “pharmaceutically active ingredient” is intended an ingredient in the composition which has a physiological, metabolic, physical, or mechanical effect when introduced into an animal (e.g., a human or veterinary animal) and is therefore useful in therapeutic and diagnostic methods for treating, ameliorating, preventing and/or diagnosing a disease or disorder in the animal into which the pharmaceutically active ingredient is introduced. Examples of suitable pharmaceutically active ingredients for use in preparing the paste formulations provided by the present invention include, but are not limited to, peptides, proteins, and small molecule therapeutic or diagnostic agents.

In certain embodiments, the pharmaceutically active ingredient is a peptide or protein therapeutic. Exemplary peptide or protein therapeutics include those that have been approved for use in human and/or veterinary animal therapeutic and/or diagnostic use, such as those therapeutic peptides and proteins listed in the online “THPdb” database (available at http://crdd.osdd.net/raghava/thpdb/). Such peptide and protein therapeutics include, but are not limited to, an enzyme (such as dornase alpha, velaglucerase alpha, taliglucerase alpha, asparaginase, glucarpidase, asfotase alpha, elosulfase alpha, sebelipase alpha, sacrosidase and pegloticase), an antithrombin agent (such as lepirudin, bivalirudin, defibrotide and sulodexide), a thrombolytic agent (such as reteplase, anistreplase, tenecteplase, streptokinase and urokinase), a peptide or protein hormone (such as parathyroid hormone, amylin, angiotensin, growth hormone, growth hormone-releasing factor, glatiramer, exenatide, insulin-like growth factor, cosinotropin, chorionic gonadotropin (e.g., human chorionic gonadotropin) and somatotropin), a bone-active peptide or protein (such as calcitonin, e.g., salmon calcitonin), a diabetic-active peptide or protein (such as insulin (which may be human or porcine) and analogues thereof (including insulin lispro, insulin glargine, insulin aspart, insulin detemir, and insulin glulisine), pramlintide, and glucagon and analogues thereof (including dasiglucagon), an antibody or a fragment thereof (which may be a monoclonal antibody or a fragment thereof such as cetuximab, trastuzumab, bevacizumab, rituximab, obinutuzumab, gemtuzumab, canakinumab, ipilimumab, daratumumab, vedolizumab, ustekinumab, siltuximab, ramucirumab, pembrolizumab, ofatumumab, nivolumab, mepolizumab, brodalumab, pertuzumab, denosumab, golimumab, belimumab, raxibacumab, blinatuomab, dinutuximab, and ibritumomab), a non-antibody antineoplastic agent (such as leuprolide, denileukin diftitox, aldesleukin, asparaginase, pegasparagase, interferon beta, afibercept, lenograstim and sipuleucel-T), a fertility agent (such as leuprolide, a menotropin, lutropin alpha, follitropin beta, urofollitropin, and choriogonadotropin alpha), and an immunosuppressive agent (such as etanercept, peginterferon alpha, an interferon alpha, filgrastim, pegfilgrastim, sargramostim, anakinra, an interferon beta, an interferon gamma, adalimumab, infliximab, basiliximab, muromonab, efalizumab, daclizumab, abatacept, rilonacept, belatacept, natalizumab, blintumomab, ustekinumab and human immune globulin). Other protein and peptide therapeutics suitable for use in the compositions and methods of the present invention will be familiar to those of ordinary skill in the art. Protein and peptide therapeutics advantageously used in accordance with the present invention may be naturally derived, synthetic or produced recombinantly, using methods of peptide and protein production that are well-known in the art.

Other active pharmaceutical ingredients suitably used in producing the compositions of the present invention by the methods of the present invention are small molecule therapeutic and/or diagnostic agents and salts thereof. Such agents are typically low molecular weight (e.g., less than about 1000 daltons) organic or inorganic compounds that have a desired bioactivity making them useful in treating, ameliorating, preventing and/or diagnosing a disease or disorder once the agent is introduced into the body of an animal (e.g., a human or a veterinary animal). Examples of such small molecule active pharmaceutical ingredients (and salts thereof) suitable for use in accordance with the present invention include, but are not limited to, epinephrine, benzodiazepines, catecholemines, “triptans,” sumatriptan, novantrone, chemotherapy small molecules (e.g., mitoxantrone), corticosteroid small molecules (e.g., methylprednisolone, beclomethasone dipropionate), immunosuppressive small molecules (e.g., azathioprine, cladribine, cyclophosphamide monohydrate, methotrexate), anti-inflammatory small molecules (e.g., salicylic acid, acetylsalicylic acid, lisofylline, diflunisal, choline magnesium trisalicylate, salicylate, benorylate, flufenamic acid, mefenamic acid, meclofenamic acid, triflumic acid, diclofenac, fenclofenac, alclofenac, fentiazac, ketorolac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin, sulindac, phenylbutazone, oxyphenbutazone, azapropazone, feprazone, piroxicam, isoxicam), small molecules used to treat neurological disorders (e.g., cimetidine, ranitidine, famotidine, nizatidine, tacrine, metrifonate, rivastigmine, selegilene, imipramine, fluoxetine, olanzapine, sertindole, risperidone, valproate semisodium, gabapentin, carbamazepine, topiramate, phenytoin), small molecules used to treat cancer (e.g., vincristine, vinblastine, paclitaxel, docetaxel, cisplatin, irinotecan, topotecan, gemcitabine, temozolomide, imatinib, bortezomib), statins (e.g., atorvastatin, amlodipine, rosuvastatin, sitagliptin, simvastatin, fluvastatin, pitavastatin, lovastatin, pravastatin, simvastatin), and other taxane derivatives, small molecules used to treat tuberculosis (e.g., rifampicin), small molecule anti-fungal agents (e.g., fluconazole, ketoconazole), small molecule anti-anxiety agents and small molecule anti-convulsant agents (e.g., lorazepam), small molecule anti-cholinergic agents (e.g., atropine), small molecule β-agonist drugs (e.g., albuterol sulfate), small molecule mast cell stabilizers and small molecule agents used to treat allergies (e.g., cromolyn sodium), small molecule anesthetic agents and small molecule anti-arrhythmic agents (e.g., lidocaine), small molecule antibiotic agents (e.g., tobramycin, ciprofloxacin), small molecule anti-migraine agents (e.g., sumatriptan), and small molecule anti-histamine drugs (e.g., diphenhydramine). Other small molecule therapeutics and diagnostics, and salts thereof, which are suitable for use in the compositions and methods of the present invention will be familiar to those of ordinary skill in the art. Additional formulations comprise combinations of such agents, comprising at least two of the small molecule therapeutics and diagnostics described herein and others that are familiar to those of ordinary skill in the art. Small molecules and salts thereof that can be advantageously used in accordance with the present invention may be obtained commercially from a wide range of sources (e.g., ThermoFisher, Aldrich Chemical and the like), or may be synthesized sing methods of chemical and biochemical synthesis that are well-known in the art.

### Methods of Use

The compositions of the present invention can be used to treat, ameliorate, prevent or diagnose a variety of diseases and physical disorders in animals, including veterinary animals or humans, in need of such treatment, amelioration, prevention and diagnosis. Suitable such methods involve the administration of one or more of the paste compositions of the present invention by injection, suitably intracutaneously, subcutaneously or intramuscularly, in relatively low volumes (e.g., 1 μL to 10000 μL or less), resulting in the delivery of a bolus of therapeutic compound in potentially lower volumes and/or more rapidly than can be achieved using other methods of administration of aqueous therapeutic formulations having lower concentrations of active ingredient, e.g., via intravenous infusion. These methods of use may result in less discomfort to the animal post-injection, and also demonstrate certain pharmacokinetic and pharmacodynamic advantages as detailed in the Examples hereinbelow. Diseases and physical disorders suitably treated, prevented, ameliorated or diagnosed using the paste compositions of the present invention will be readily apparent to those of ordinary skill in the art, and the choice of active ingredient to be used as a starting material in producing the pastes of the invention will also be familiar to the ordinarily skilled practitioner based on the disease, physical disorder or condition to be treated, prevented, ameliorated, or diagnosed using the paste compositions of the present invention.

In some embodiments, an exemplary such method of the present invention comprises treating or preventing hypoglycemia by administering to a subject having hypoglycemia or at risk for experiencing hypoglycemia a paste formulation or composition as described herein in an amount effective to treat or prevent the hypoglycemia. In some embodiments, the subject is administered a paste formulation comprising glucagon. In certain aspects hypoglycemia can be caused by, or the patient can be at higher risk for experiencing hypoglycemia because of, diabetes or non-diabetes related diseases, conditions, and disorders.

As described by the Workgroup of the American Diabetes Association and the Endocrine Society (Seaquist et al., Diabetes Care 36: 1384-1395 (2013)) with respect to hypoglycemia, a single threshold value for plasma glucose concentration that defines hypoglycemia in diabetes is not typically assigned because glycemic thresholds for symptoms of hypoglycemia (among other responses) shift to lower plasma glucose concentrations after recent antecedent hypoglycemia and to higher plasma glucose concentrations in patients with poorly controlled diabetes and infrequent hypoglycemia.

Nonetheless, an alert value can be defined that draws the attention of both patients and caregivers to the potential harm associated with hypoglycemia. Patients at risk for hypoglycemia (i.e., those treated with a sulfonylurea, glinide, or insulin) should be alert to the possibility of developing hypoglycemia at a self-monitored plasma glucose—or continuous glucose monitoring subcutaneous glucose—concentration of ≤70 mg/dL (≤3.9 mmol/L). Because it is higher than the glycemic threshold for symptoms in both nondiabetic individuals and those with well-controlled diabetes, it generally allows time to prevent a clinical hypoglycemic episode and provides some margin for the limited accuracy of monitoring device at low-glucose levels.

The condition of severe hypoglycemia is an event requiring assistance of another person to actively administer carbohydrates, glucagon, or take other corrective actions. Plasma glucose concentrations may not be available during an event, but neurological recovery following the return of plasma glucose to normal is considered sufficient evidence that the event was induced by a low plasma glucose concentration. Typically, these events begin occurring at plasma glucose concentrations of ≤50 mg/dL (2.8 mmol/L). Documented symptomatic hypoglycemia is an event during which typical symptoms of hypoglycemia are accompanied by a measured plasma glucose concentration ≤70 mg/dL (≤3.9 mmol/L). Asymptomatic hypoglycemia is an event not accompanied by typical symptoms of hypoglycemia but with a measured plasma glucose concentration ≤70 mg/dL (≤3.9 mmol/L). Probable symptomatic hypoglycemia is an event during which symptoms typical of hypoglycemia are not accompanied by a plasma glucose determination but that was presumably caused by a plasma glucose concentration ≤70 mg/dL (≤3.9 mmol/L). Pseudo-hypoglycemia is an event during which the person with diabetes reports any of the typical symptoms of hypoglycemia with a measured plasma glucose concentration >70 mg/dL (>3.9 mmol/L) but approaching that level.

Further included in the indications which may be treated by the disclosed invention are hypoglycemia-associated autonomic failure (HAAF). As described by Philip E. Cryer, Perspectives in Diabetes, Mechanisms of Hypoglycemia-Associated Autonomic Failure and Its Component Syndromes in Diabetes, Diabetes, Vol. 54, pp. 3592-3601 (2005), “recent antecedent iatrogenic hypoglycemia causes both defective glucose counter-regulation (by reducing epinephrine responses to a given level of subsequent hypoglycemia in the setting of absent decrements in insulin and absent increments in glucagon) and hypoglycemia unawareness (by reducing sympathoadrenal and the resulting neurogenic symptom responses to a given level of subsequent hypoglycemia) and thus a vicious cycle of hypoglycemia.” HAAF affects those with type 1 and advanced type 2 diabetes. Additionally, the invention of the present disclosure may also treat hypoglycemia in patients following islet cell transplantation.

The compositions of the present invention can also be used for the treatment or prevention of hyperinsulinemic hypoglycemia, which broadly refers to the condition and effects of low blood glucose levels that are caused by excessive insulin. The most common type of severe, but typically transient, hyperinsulinemic hypoglycemia arises from the administration of exogenous insulin in patients with Type 1 diabetes. This type of hypoglycemia can be defined as iatrogenic hypoglycemia and is a limiting factor in the glycemic management of type 1 and type 2 diabetes. Nocturnal hypoglycemia (night-time hypo) is a common type of iatrogenic hypoglycemia arising in patients taking exogenous insulin. However, hyperinsulinemic hypoglycemia can also arise due to endogenous insulin, for example in congenital hyperinsulinism, insulinomas (insulin-secreting tumors), exercise-induced hypoglycemia and reactive hypoglycemia. Reactive hypoglycemia is a non-diabetic hypoglycemia and is due to low blood sugar that occurs following a meal—typically within four hours after eating. Reactive hypoglycemia may also be referred to as postprandial hypoglycemia. Symptoms and signs of reactive hypoglycemia can include hunger, weakness, shakiness, sleepiness, sweating, confusion and anxiety. Stomach surgery (e.g. bariatric surgery) is one possible cause, as following surgery food may pass too quickly into the small intestine (e.g. post-bariatric hypoglycemia (PBH)). Additional causes include enzyme deficiencies that make it difficult for the body to breakdown food, or increased sensitivity to the hormone epinephrine.

In some embodiments, the disease, condition, or disorder to be treated or prevented with a paste composition of the present invention is a diabetic condition. Examples of diabetic conditions include, but are not limited to, type 1 diabetes, type 2 diabetes, gestational diabetes, pre-diabetes, hyperglycemia, hypoglycemia, and metabolic syndrome. In some embodiments, the disease, condition, or disorder is hypoglycemia, including but not limited to diabetes-related hypoglycemia, exercise-induced hypoglycemia, and post-bariatric surgery hypoglycemia, or other types of hypoglycemia described herein and known to those of ordinary skill in the art. In some embodiments, the disease, condition, or disorder is diabetes.

In some embodiments, a method of the present invention comprises treating diabetes by administering to a subject having diabetes a therapeutic agent in a paste formulation as described herein in an amount effective to treat the diabetes. In some embodiments, the subject is administered a paste formulation comprising insulin. In some embodiments, the subject is administered a paste formulation comprising pramlintide. In some embodiments, the subject is administered a paste formulation comprising insulin and pramlintide. In some embodiments, the subject is administered a paste formulation comprising exenatide. In some embodiments, the subject is administered a paste formulation comprising glucagon and exenatide.

In certain aspects a paste formulation of the invention comprising epinephrine can be administered to a subject at risk of or suspected of anaphylaxis. Epinephrine is indicated as an emergency treatment of Type I allergic reactions which can arise from multiple sources, including, but not limited to, foods, drugs and/or other allergens, allergen immunotherapy, diagnostic testing substances, insect stings and bites, and idiopathic or exercise-induced anaphylaxis.

Other diseases, disorders and conditions suitably treated, prevented, ameliorated or diagnosed using the compositions and methods of the present invention will be readily familiar to the ordinarily skilled artisan and include, without limitation, cancers, infectious diseases, bacterial diseases, fungal diseases, viral diseases, and other diseases, disorders and conditions involving inflammatory, neurological, osteological, gastrointestinal, circulatory, cardiovascular, skin, muscular, developmental and other symptoms, signs or dysfunctions.

The present invention has been described herein by reference to illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. It will be readily apparent to one of ordinary skill in the relevant arts that other suitable modifications and adaptations to the methods and applications described herein may be made without departing from the scope of the invention or any embodiment thereof. Similarly, it will be understood that the benefits and advantages described above may relate to one embodiment or may relate to several embodiments. Having now described the present invention in detail, the same will be more clearly understood by reference to the following examples, which are included herewith for purposes of illustration only and are not intended to be limiting of the invention.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that changes can be made to the specific embodiments which are disclosed herein without departing from the spirit and scope of the invention.

### Example 1

### Manufacturing Process Effects on Stability of Spray-Dried Powders Containing Proteins

Earlier work by some of the present inventors established initial processes for preparing injectable paste formulations containing relatively high concentrations of certain proteins and peptides (see, e.g., U.S. Pat. Nos. 8,790,679; 8,110,209; and 9,314,424; and U.S. Patent Publication Nos. US 2017/0007675 and US 2017/0216529, the disclosures of all of which are incorporated herein by reference in their entireties). In the present study, the effects of various manufacturing parameters on the preparation and stability of spray-dried powders and pastes containing high concentrations of antibodies was examined, with the goal to optimize the manufacturing process for use in preparation of storage-stable commercial formulations comprising high concentrations of therapeutic active ingredients, particularly high molecular weight proteins such as antibodies.

Different paste formulations containing spray dried powders containing an IgG antibody were initially prepared by spray-drying aqueous IgG solutions (e.g., aqueous ‘feed solutions’ containing 20 mg/mL of IgG) that contained various carriers and excipients and at differing pH values (as measured prior to spray drying). Details are shown in Table 2 below.

TABLE 2
Representative Powder Compositions

| Formulation 1 | | Formulation 2 | | |
|---|---|---|---|---|
| Component | Concentration | Component | Concentration | |
| IgG | 20 mg/mL | IgG | 20 | mg/mL |
| Trehalose | 2.5 mg/mL | Trehalose | 5.15 | mg/mL |
| Na⁺/K⁺, Ca⁺⁺, | Total <0.8 | Polysorbate 20 | 0.05 | mg/mL |
| Cl⁻ | mg/mL | | | |
| | | Methionine | 0.412 | mg/mL |
| | | Citrate | 0.192 | mg/mL |

| | | | |
|---|---|---|---|
| pH | 6.3 | pH | 4.0 |

The two formulations were then spray dried using the following settings and under the conditions shown in Table 3.

TABLE 3
Spray Drying Conditions for two Exemplary Formulations

| | Formulation 1 | | Formulation 2 | |
|---|---|---|---|---|
| Inlet temperature | 90° C. | | 70° C. | |
| Aspirator settings | 65% | (27 m3/hr) | 85% | (34 m3/hr) |
| Nozzle gas rate | 40 mm (473 L/hr, | | 40 mm (473 L/hr, | |
| | 667 L/hr, STP | | 667 L/hr, STP | |
| Feed solution | 3% | (1 mL/min) | 10% | (~3 mL/min) |
| pump rate | | | | |

| | | |
|---|---|---|
| Comments | No secondary drying or | Secondary drying and |
| | sieving after spray | sieving after spray |
| | drying | drying (150 mTorr, |
| | | 5° C., 2 days) |

Unless otherwise specified, all spray drying studies discussed in this application were performed using the B-290 mini-spray dryer manufactured by BUCHI Corporation equipped with the standard two-fluid nozzle. This spray dryer includes a built-in floating ball flowmeter for the nozzle (atomizing) gas flow rate on a scale of 0-60 mm. Aspirator air flow rates and liquid feed pump settings are input on a scale of 0-100% with the B-290 instrument, with the conversion to standard units of air flow (L/hr) and liquid flow (mL/min) provided in the B-290 operating manual.

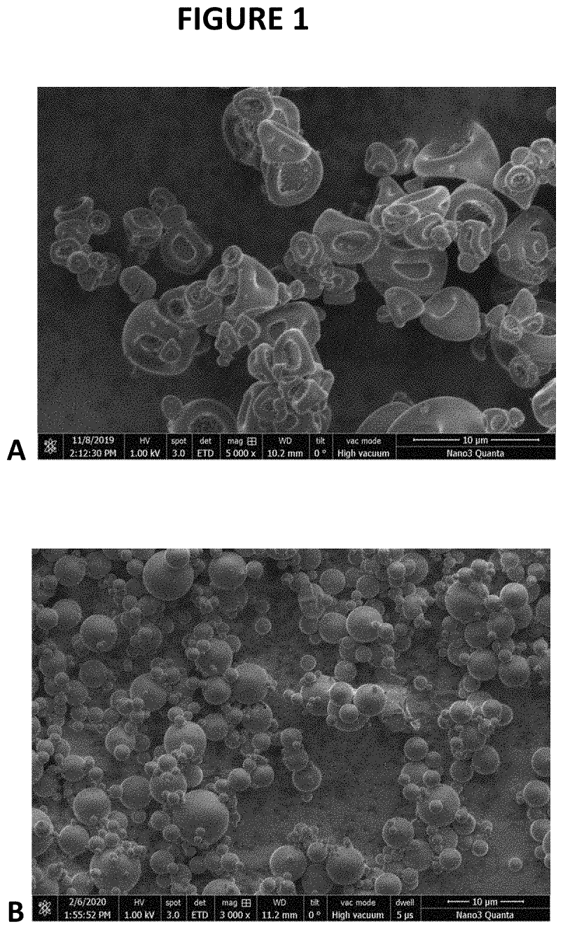

Following preparation of these two spray dried powder formulations, samples were subjected to scanning electron microscopy (SEM) to examine the morphology of the particles of the two formulations. As seen in FIG. 1, SEM micrographs of the two formulations showed distinct differences in the particle morphologies. The particles of Formulation 1 were extensively pitted and showed an irregular shape, while those of Formulation 2 demonstrated much more regular, spherical shape with no apparent pitting. In addition, on average the particles in Formulation 1 were somewhat larger than those in Formulation 2. These physical characteristics together indicate that the particles of Formulation 1 had a higher surface area than those of Formulation 2.

Pastes were then prepared from these two formulations by adding Miglyol 812 N to them to the point where a paste composition was obtained but prior to conversion of the paste to a suspension, where the relative excess of liquid to solid phase would enable particle settling over time. For Formulation 1 the solids content range where a paste was formed was considerably lower than the range where a paste formed for Formulation 2. Formulation 1 formed a paste in range of approximately 38%-42% w/w solids content, representing a solids concentration of 475 mg/mL solids and an IgG content of approximately 400 mg/mL. In contrast, for formulation 2, a paste containing 65% w/w solids content was prepared, representing approximately 820 mg/mL solids concentration and an IgG concentration of approximately 630 mg/mL. These results suggest that the physical properties of the spray dried powder of Formulation 1 (e.g., larger surface area due to surface rugosity) yielded a lower-concentration paste (both in terms of solids concentration and IgG concentration) for Formulation 1 relative to Formulation 2.

To evaluate the influence of solids content on the injection force needed to deliver the paste through a hypodermic needle (modeling injection of therapeutic paste formulations into an animal), 1 mL of these pastes were loaded into glass syringes (internal diameter ˜4.6 mm) and delivered through 27 G ultrathin wall ¼ inch (˜6 mm exposed length) needle affixed to the syringes via a Luer-lock fitting. The force required to deliver the 1 mL paste in 30 seconds (e.g., 33.3 μL/sec volumetric flow rate) from the syringes was measured using a texture analyzer (force is plotted against the plunger distance traveled). Formulation 1 (42% w/w solids, ˜400 mg/mL IgG) required an injection force of about 36N to expel 1 mL of paste in 30 seconds, while an injection force of approximately 60N was required to expel Formulation 2 (65% w/w solids, 630 mg/mL IgG) reflecting in part their differences in overall solids content

A lower injection force may facilitate delivery and improve overall ease-of-administration. Therefore, it would be desirable to produce high active ingredient (e.g., mAb) concentration paste formulations that can be delivered using commercially available syringe/needle combinations at relatively low injection forces. To evaluate the impact of other spray-drying process parameters on the ability to produce stable syringeable therapeutic protein paste formulations, the inventors evaluated the level of protein aggregation in a non-specific IgG formulation pre- and post-spray-drying at different pHs of the initial solution. Aqueous feed solutions of IgG at 20.0 mg/mL were prepared in the “Formulation 2” solution described above in Table 3, but at either pH 4 (using a citrate buffer) or pH 6 (using a histidine buffer). The relative percentage of mAb aggregates post-spray-drying was then determined using size exclusion chromatography. In both formulations, the aggregation level after spray-drying was approximately 2.6% for the pH 6 formulation and 1.9% for the pH 4 formulation, indicating the pH of the starting feed solution may promote a measurable difference in % protein aggregation of the finished spray-dried powder. For certain IgG formulations, the preferred pH for minimizing aggregate formation may be in the range of 3.5-4.0.

To further evaluate the impact of the spray-drying process parameters themselves, a series of experiments were conducted to examine the effects of inlet temperature, aspirator flow rate, and nozzle pressure gas flow rate during the spray-drying process of IgG solutions on the resulting morphology (sphericity), size and size distribution of IgG particles in the resulting spray-dried powder. Eight sets of process parameters were evaluated (Table 4), using a non-specific IgG starting solution at 20 mg/mL in the Formulation 2 solution noted above with a pH of 6.0. All samples were filtered prior to spray-drying, and then subjected to secondary drying under vacuum.

TABLE 4
Spray-Drying Process Parameters

| Formulation | Inlet Temp | Aspirator | Gas Flow Rate | Feed Flow Rate |
|---|---|---|---|---|
| No. | (° C.) | (%) | (mm) | (%) |
| 1 | 90 | 100 | 40 | 10 |
| 2 | 90 | 85 | 60 | 10 |
| 3 | 90 | 85 | 40 | 10 |
| 4 | 90 | 100 | 60 | 10 |
| 5 | 70 | 100 | 40 | 10 |
| 6 | 70 | 85 | 40 | 10 |
| 7 | 70 | 100 | 60 | 10 |
| 8 | 70 | 85 | 60 | 10 |

Following spray-drying, the resultant powders were evaluated visually for appearance, and were then solubilized (dissolved) at 1 mg/mL in water-for-injection (WFI) and evaluated for dissolution time, as well as percentage of aggregation via size exclusion chromatography (SEC). Results are shown in Table 5.

TABLE 5
Effects of Spray-Drying Parameters on Appearance of IgG Powders

| | | | | | IgG | |
|---|---|---|---|---|---|---|
| | | | Gas | Feed | Powder | |
| | Inlet | | Flow | Flow | Dissolution | |
| Formulation | Temp | Aspirator | Rate | Rate | Time | SEC % |
| No. | (C.) | (%) | (mm) | (%) | (1 mg/mL) | Aggregate |
| 1 | 90 | 100 | 40 | 10 | 22-27 | 6.42 |
| 2 | 90 | 85 | 60 | 10 | seconds | 6.35 |
| 3 | 90 | 85 | 40 | 10 | | 6.64 |
| 4 | 90 | 100 | 60 | 10 | | 6.42 |
| 5 | 70 | 100 | 40 | 10 | | 6.28 |
| 6 | 70 | 85 | 40 | 10 | | 6.31 |
| 7 | 70 | 100 | 60 | 10 | | — |
| 8 | 70 | 85 | 60 | 10 | | 6.3 |

As seen in Table 5, powders prepared from the different feed solutions evaluated in this study contained a similar percentage of measured protein aggregation upon dissolution, with each of the powders also demonstrating approximately similar dissolution times in the close range of 22-27 seconds.

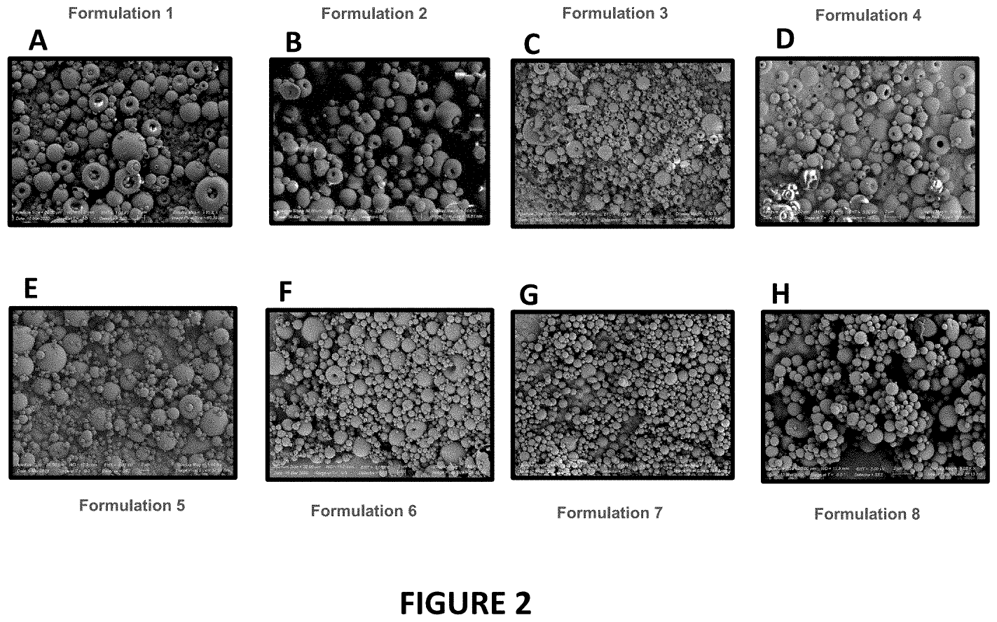

To more closely examine these powders at the particle level, a sample of powder from each of the eight formulations described in Table 5 was examined using scanning electron microscopy (SEM), to evaluate particle size, particle shape, and the distribution of each in a given sample. Results are shown in FIG. 2.

As seen in FIGS. 2A-2D, formulations 5-8, all of which used a spray-dryer inlet temperature of 90° C., exhibited a variety of particle sizes and particle morphologies, with a mixture of small and large particles demonstrating a mixture of spherical and toroidal shapes. In contrast, as seen in FIGS. 2E-2H, Formulations 5-8, all of which used a spray-dryer inlet temperature of 70° C., showed more uniform particles in terms of size and shape, with most particles appearing to be spherical and only a very small number of toroid particles observed. Of these formulations, Formulations 7 and 8 appeared to be the most uniform in size, demonstrating a preponderance of small spherical particles.

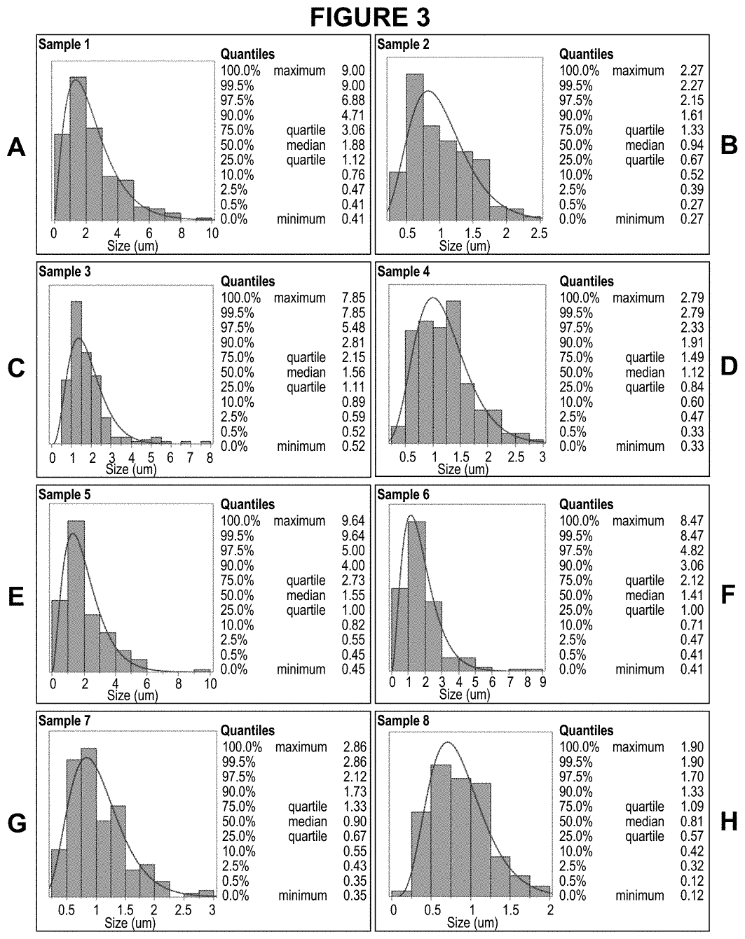

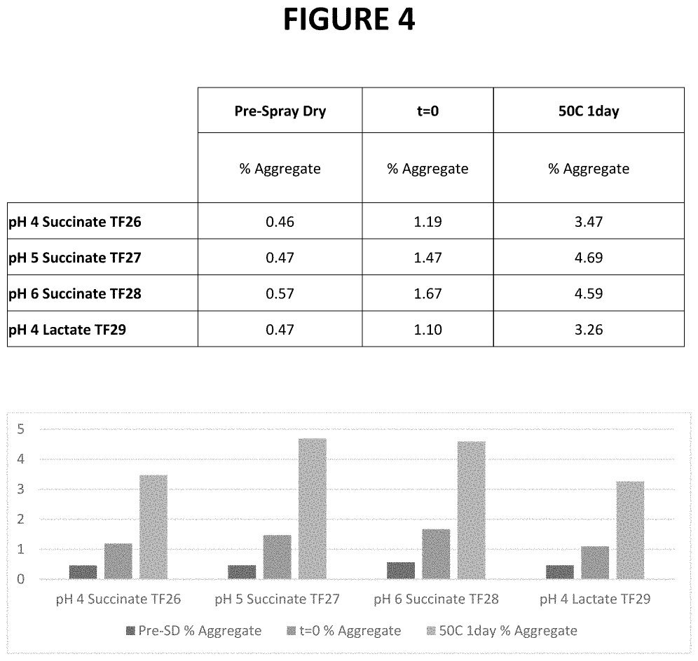

These results were confirmed when particle size distribution was measured, by individually measuring the diameter (on representative SEM photomicrographs) of about 200 IgG particles per formulation; results of these measurements are shown in FIGS. 3-4. As seen in FIG. 3, Formulations 1-4 which used a spray-dryer inlet temperature of 90° C. exhibited a larger particle size distribution (FIGS. 3A-3D) compared to Formulations 5-8 which used a spray-dryer inlet temperature of 70° C. (FIGS. 3E-3H). Interestingly, at a given spray-dryer inlet temperature, a higher gas flow rate tended to favor the production of smaller and more uniform particles; compare, for example, those formulations dried at a 40 mm gas flow rate (FIGS. 3A, 3C, 3E and 3F) to their corresponding formulations dried at a 60 mm gas flow rate (FIGS. 3B, 3D, 3G and 3H, respectively).

Taken together, the results of these studies indicate that the spray-drying settings can influence the production of spherical particles with the appropriate size distribution. In particular, an inlet temperature of about 70-90° C., and more particularly about 70-80° C., and a gas flow rate of about 40-60 mm, and more particularly about 60 mm, appeared to produce smaller and more spherical particles in the powder. In fact, it was determined that on the B-290 spray dryer an inlet temperature of about 70° C. is the lowest temperature that can be used to obtain spherical particles; using a lower temperature with the feed solution and process parameters described in this Example, with the expectation that the protein or peptide might be less prone to temperature-induced denaturation, actually had the effect of producing more toroidal particles of nonuniform size distribution (data not shown). The ability to produce smaller spherical particles with an appropriate size distribution is important for the ultimate production of high solids content, high protein concentration pastes that may be suitably injected in relatively small volumes into animals, particularly humans, for therapeutic and diagnostic purposes.

### Example 2

### Effects of Formulation Excipients on Production and Injectability of Spray-Dried Proteins

Beyond the spray-dryer settings, results from the present inventors indicated that the components of the formulation that is spray-dried can have an impact on the physical and performance properties of the spray-dried powder prepared from a given formulation. To further examine and optimize these formulation component effects, a representative monoclonal antibody (trastuzumab or “TmAb”, the API in the commercial drug product Herceptin®) was formulated in the presence of a variety of excipients and buffering agents, pharmaceutically acceptable carriers or bulking agents, surfactants, etc., and the impact of each excipient on the level of aggregation of the formulation both at time 0 after powder manufacture by spray-drying and upon storage of the powder was evaluated. TmAb is a protein that in monomeric form is 148 kDa in size, but upon aggregation it forms dimers and other multimers of larger molecular weight which are themselves not only immunogenic but also serve as nucleation centers for the formation of even larger aggregates in solution; such aggregates can be immunogenic and/or be cleared by the immune system before the antibody has a chance to exert it therapeutic effect. Therefore, it would be ideal from a therapeutic standpoint to be able to prepare powders (e.g., via spray drying) containing TmAb and having low levels of aggregation upon storage, which can then be suitably used in high solids content/high concentration pastes for injection into animals including humans.

For preparation, a commercial TmAb solution was dialyzed (50 kDa molecular weight cutoff) against the desired formulation buffer (see table below) overnight at 4° C. with constant stirring. The dialyzed TmAb solution was then spray-dried using an inlet temperature of 70° C., a nozzle flow rate of 40 mm, 85% aspirator setting and 10% feed pump (about 3 mL/min). Once the powder had been produced, to lower the moisture content to a target level of <1% (w/w) the powder was secondarily dried (e.g., under vacuum) at 150 mT, 5° C. for one day, then at 30° C. for three hours, and the powder was then stored in glass vials backfilled with nitrogen and stoppered to produce a closed system. Samples were stored at 40° C. for four days or at 50° C. for four hours and compared to t=0 samples (immediately after lyophilization) for degree of aggregation and other stability parameters by solubilizing the powder sample to a concentration of 1 mg/mL TmAb in water and then analyzing the solutions by size exclusion, ion exchange and reverse phase chromatography.

In a first round of experiments, five formulations were prepared each containing 20 mg/mL TmAb, trehalose at 5.15 mg/mL, polysorbate 20 at 0.05 mg/mL, methionine at 0.412 mg/mL, as well as the remaining excipients and at the pH adjusted to the values shown in Table 6.

TABLE 6
pH and Buffer Species Formulations (Round 1)

| | Feed | | Feed | | Feed | | Feed | | Feed | |
|---|---|---|---|---|---|---|---|---|---|---|
| | Soln. | Powder | Soln. | Powder | Soln. | Powder | Soln. | Powder | Soln. | Powder |
| | Batch | Batch | Batch | Batch | Batch | Batch | Batch | Batch | Batch | Batch |
| | TF2 | TF2 | TF3 | TF3 | TF4 | TF4 | TF5 | TF5 | TF6 | TF6 |
| TmAb | 20.0 | 77.51% | 20.0 | 77.65% | 20.0 | 77.65% | 20.0 | 77.81% | 20.0 | 77.81% |
| | mg/mL | | mg/mL | | mg/mL | | mg/mL | | mg/mL | |
| TmAb | — | 565.4 | — | 572.9 | — | 572.9 | — | 574.1 | — | 574.1 |
| mg/mL | | mg/mL | | mg/mL | | mg/mL | | mg/mL | | mg/mL |
| in paste | | | | | | | | | | |
| (assume | | | | | | | | | | |
| 60% w/w | | | | | | | | | | |
| load) | | | | | | | | | | |
| Citrate | 0.192 | 0.74% | — | — | — | — | — | — | — | — |
| | mg/mL | | | | | | | | | |
| Adipate | — | — | 0.146 | 0.57% | 0.146 | 0.57% | — | — | — | — |
| | | | mg/mL | | mg/mL | | | | | |
| Lactate | — | — | — | — | — | — | 0.090 | 0.35% | 0.090 | 0.35% |
| | | | | | | | mg/mL | | mg/mL | |
| pH | 4.0 | — | 4.0 | — | 5.0 | — | 4.0 | — | 5.0 | — |
| Total | 25.80 | 100% | 25.76 | 100% | 25.76 | 100% | 25.70 | 100% | 25.70 | 100% |
| | mg/mL | | mg/mL | | mg/mL | | mg/mL | | mg/mL | |

These formulations were then evaluated for level of protein aggregation in the pre-spray-dried formulation and in the solubilized spray-dried powder, via size exclusion chromatography. Results are shown in Table 7.

TABLE 7
Effect of pH and Buffer Species on TmAb Aggregation (Round 1)

| | % Aggregation, Pre- | % Aggregation, Post- |
|---|---|---|
| | spray-drying | spray-drying |
| pH 4.0, Citrate (TF2) | 0.48 | 0.61 |
| pH 4.0, Adipate (TF3) | 0.46 | 0.62 |
| pH 5.0, Adipate (TF4) | 0.46 | 0.63 |
| pH 4.0, Lactate (TF5) | 0.49 | 0.67 |
| pH 5.0, Lactate (TF6) | 0.54 | 0.66 |

To expand upon these studies, a second round of formulations were prepared containing the same levels of TmAb, trehalose and polysorbate 80 as described for Round 1 above, but containing either succinate or lactate buffer and at a pH of 4-6, as shown in Table 8:

TABLE 8
pH and Buffer Species Formulations (Round 2)

| | Feed | | Feed | | Feed | | Feed | |
|---|---|---|---|---|---|---|---|---|
| | Soln. | Powder | Soln. | Powder | Soln. | Powder | Soln. | Powder |
| | Batch | Batch | Batch | Batch | Batch | Batch | Batch | Batch |
| | TF26 | TF26 | TF27 | TF27 | TF28 | TF28 | TF29 | TF29 |
| TmAb | 20.0 | 77.51% | 20.0 | 77.65% | 20.0 | 77.65% | 20.0 | 77.81% |
| | mg/mL | | mg/mL | | mg/mL | | mg/mL | |
| TmAb | — | 562.4 | — | 562.4 | — | 562.4 | — | 565.4 |
| mg/mL | | mg/mL | | mg/mL | | mg/mL | | mg/mL |
| in paste | | | | | | | | |
| (assume | | | | | | | | |
| 60% w/w | | | | | | | | |
| load) | | | | | | | | |
| Sucinate | 0.590 | 2.25% | 0.590 | 2.25% | 0.590 | 2.25% | — | — |
| | mg/mL | | mg/mL | | mg/mL | | | |
| Lactate | — | — | — | — | — | — | 0.450 | 1.72% |
| | | | | | | | mg/mL | |
| pH | 4.0 | — | 5.0 | — | 6.0 | — | 4.0 | — |
| Total | 26.24 | 100% | 26.24 | 100% | 26.24 | 100% | 26.10 | 100% |
| | mg/mL | | mg/mL | | mg/mL | | mg/mL | |

These formulations were then evaluated for level of protein aggregation in the pre-spray-dried formulation and in the solubilized spray-dried powder, via size exclusion chromatography, comparing samples tested pre-spray-drying and those pulled at t=0 after spray-drying and others stored at 50° C. for one day. Results are shown in FIG. 4. Together with those from the Round 1 formulations, these results indicate that the optimal buffer and pH conditions for the pre-spray-dry solution, in order to minimize the amount of aggregation in both the pre-sprayed solution and in the post-sprayed and dried powder, is the use of a lactate buffer and a pH of from about 3.5 to about 4.5, e.g., about 4.0, particularly for IgG-containing formulations.

Next, the impact of trehalose amounts in the pre-spray-dry formulation on the aggregation in solution and in the post-spray-dried powder was evaluated. TmAb formulations (20 mg/mL) in a 5 mM lactate buffer, pH 4.0 or 6.0, were prepared, containing the amounts of trehalose and other excipients shown in Table 9:

TABLE 9
Formulations with varying sugar content

| | Feed | | Feed | | Feed | |
|---|---|---|---|---|---|---|
| | Soln. | Powder | Soln. | Powder | Soln. | Powder |
| | Batch | Batch | Batch | Batch | Batch | Batch |
| | TF30 | TF30 | TF31 | TF31 | TF32 | TF32 |
| TmAb | 20.0 | 76.63% | 20.0 | 64.62% | 20.0 | 55.63% |
| | mg/mL | | mg/mL | | mg/mL | |
| TmAb | — | 565.4 | — | 476.8 | — | 410.4 |
| mg/mL | | mg/mL | | mg/mL | | mg/mL |
| in paste | | | | | | |
| (assume | | | | | | |
| 60% w/w | | | | | | |
| load) | | | | | | |
| Trehalose | 5.15 | 19.73% | 10.0 | 32.31% | 15 | 41.72% |
| | mg/mL | | mg/mL | | mg/ml | |
| Polysorbate | 0.5 | 1.92% | 0.5 | 1.62% | 0.5 | 1.39% |
| 20 | mg/mL | | mg/mL | | mg/mL | |
| Lactate | 0.45 | 1.72% | 0.45 | 1.45% | 0.45 | 1.25% |
| | mg/mL | | mg/mL | | mg/mL | |
| pH | 4.0 | — | 4.0 | — | 4.0 | — |
| Total | 26.10 | 100% | 30.95 | 100% | 35.95 | 100% |
| | mg/mL | | mg/mL | | mg/mL | |
| | | | | | Feed Soln. | Powder |
| | Feed | | Feed | | Batch TF25 | Batch TF25 |
| | Soln. | Powder | Soln. | Powder | (Batch TF7, | (Batch TF7, |
| | Batch | Batch | Batch | Batch | post-SD | post-SD |
| | TF7 | TF7 | TF13 | TF13 | lyophilized) | lyophilized) |
| TmAb | 20.0 | 51.28% | 20.0 | 32.88% | 20.0 | 51.28% |
| | mg/mL | | mg/mL | | mg/mL | |
| TmAb | — | 378.4 | — | 242.6 | — | 378.4 |
| mg/mL | | mg/mL | | mg/mL | | mg/mL |
| in paste | | | | | | |
| (assume | | | | | | |
| 60% w/w | | | | | | |
| load) | | | | | | |
| Trehalose | 18.18 | 46.61% | 40 | 65.76% | 18.18 | 46.61% |
| | mg/mL | | mg/mL | | mg/mL | |
| Polysorbate | 0.081 | 0.21% | 0.081 | 0.13% | 0.081 | 0.21% |
| 20 | mg/mL | | mg/mL | | mg/mL | |
| Histidine | 0.743 | 1.90% | 0.743 | 1.22% | 0.743 | 1.90% |
| | mg/mL | | mg/mL | | mg/mL | |
| pH | 6.0 | — | 6.0 | — | 6.0 | — |
| Total | 39.00 | 100% | 60.82 | 100% | 39.00 | 100% |
| | mg/mL | | mg/mL | | mg/mL | |

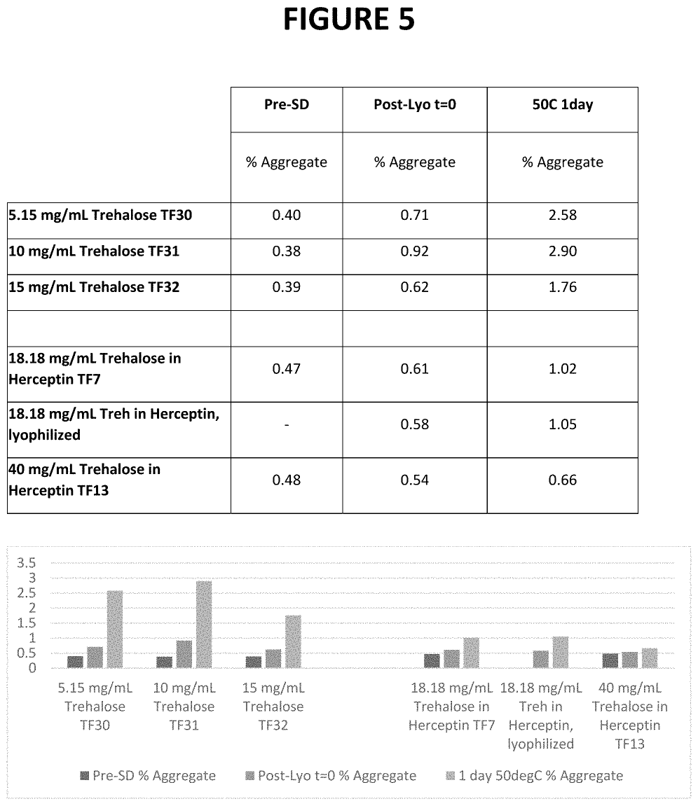

These formulations were then evaluated for level of protein aggregation in the pre-spray-dried aqueous formulation (feed solution) and in the powder post-spray drying, via size exclusion chromatography, comparing samples tested pre-spray-drying and those pulled at t=0 after spray-drying and lyophilization, and others stored at 50° C. for one day. Results are shown in FIG. 5. These results indicate that the trehalose has a stabilizing effect upon spray-drying and post-spray-drying storage, in terms of reducing the aggregation of the powder. However, relatively high amounts of trehalose are required in the formulation to achieve such stability, which thereby reduces the active content (in this case, TmAb) in the paste formulation. Thus, the amount of trehalose to be included must be optimized along with other excipients and formulation components in order to enhance stability while also permitting a relatively high active ingredient content to be included in the paste formulations. The present inventors also have results suggesting that for the formulations prepared under the conditions of this Example, using sucrose in place of trehalose, at similar concentrations, may provide a greater stabilization effect on the powdered formulations. Other excipients that can be advantageously used in a similar way include amino acids, advantageously one or more naturally occurring amino acids, such as hydrophobic amino acids (which may help prevent the hydrophobic core of one TmAb molecule from binding to a second TmAb, thus reducing aggregation), acidic/basic amino acids such as arginine which has a reported ability to stabilize protein and peptide formulations, even in the dry state, and a combination of sugars such as dextran/trehalose coformulations.

Continuing with these studies, the effect of polysorbate 20 content in the pre-spray-dry formulation on the aggregation in the feed solution and in the post-spray-dried powder was evaluated. In the initial round of these studies, TmAb formulations (20 mg/mL) in a 4.8 mM histidine buffer at pH 6.0 were prepared, containing the polysorbate 20 and other excipient contents shown in Table 10:

TABLE 10
Evaluation of Polysorbate 20 content

| | Feed | | Feed | | Feed | | Feed | |
|---|---|---|---|---|---|---|---|---|
| | Solution | Powder | Solution | Powder | Solution | Powder | Solution | Powder |
| | Batch | Batch | Batch | Batch | Batch | Batch | Batch | Batch |
| | TF20 | TF20 | TF22 | TF22 | TF23 | TF23 | TF24 | TF24 |
| TmAb | 20.0 | 75.78% | 20.0 | 76.94% | 20.0 | 71.70% | 20.0 | 66.90% |
| | mg/mL | | mg/mL | | mg/mL | | mg/mL | |
| TmAb | — | 559.1 | — | 567.7 | — | 529.0 | — | 493.6 |
| mg/mL | | mg/mL | | mg/mL | | mg/mL | | mg/mL |
| in paste | | | | | | | | |
| (assume | | | | | | | | |
| 60% w/w | | | | | | | | |
| load) | | | | | | | | |
| Trehalose | 5.15 | 19.51% | 5.15 | 19.81% | 5.15 | 18.46% | 5.15 | 17.23% |
| | mg/mL | | mg/mL | | mg/mL | | mg/mL) | |
| Polysorbate | 0.5 | 1.89% | 0.1 | 0.38% | 2 | 7.17% | 4 | 13.38% |
| 20 | mg/mL | | mg/mL | | mg/mL | | mg/mL | |
| Histidine | 0.743 | 2.82% | 0.743 | 2.86% | 0.743 | 2.66% | 0.743 | 2.49% |
| | mg/mL | | mg/mL | | mg/mL | | mg/mL | |
| pH | 6.0 | — | 6.0 | — | 6.0 | — | 6.0 | — |
| Total | 26.39 | 100% | 25.99 | 100% | 27.89 | 100% | 29.89 | 100% |
| | mg/mL | | mg/mL | | mg/mL | | mg/mL | |

Other formulations containing either Polysorbate 20 or Polysorbate 80 were also prepared as shown in Table 11:

TABLE 11
Polysorbate Formulations (continued)

| | Feed Solution | Powder Batch | Feed Solution | Powder Batch |
|---|---|---|---|---|
| | Batch TF13 | TF13 | Batch TF14 | TF14 |
| TmAb | 20.0 mg/mL | 32.88% | 20.0 mg/mL | 32.88% |
| TmAb mg/mL in | — | 242.6 mg/mL | — | 242.6 mg/mL |
| paste (assume | | | | |
| 60% w/w load) | | | | |
| Trehalose | 40 mg/mL | 65.76% | 40 mg/mL | 65.76% |
| Polysorbate 20 | 0.081 mg/mL | 0.13% | — | — |
| Polysorbate 80 | — | — | 0.081 mg/mL | 0.13% |
| Histidine | 0.743 mg/mL | 1.22% | 0.743 mg/mL | 1.22% |
| pH | 6.0 | — | 6.0 | — |
| Total | 60.82 mg/mL | 100% | 60.82 mg/mL | 100% |

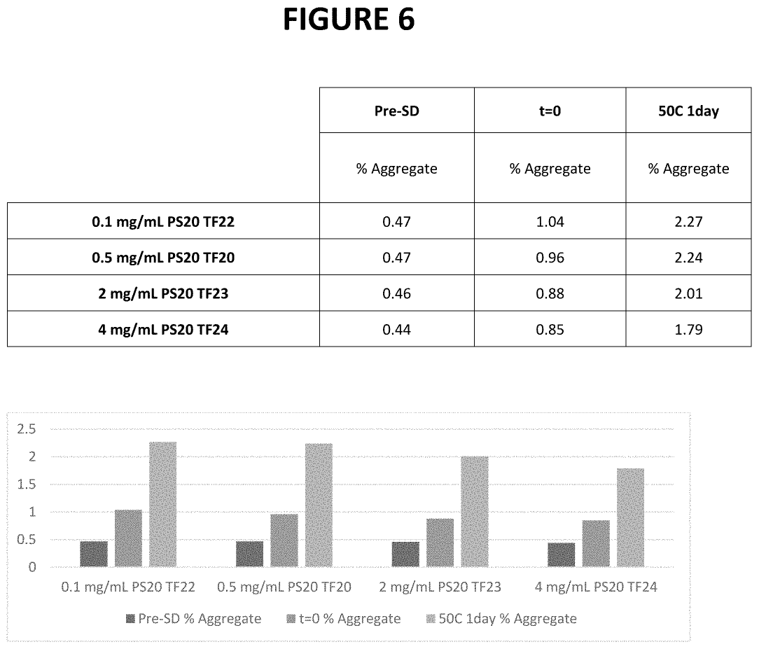

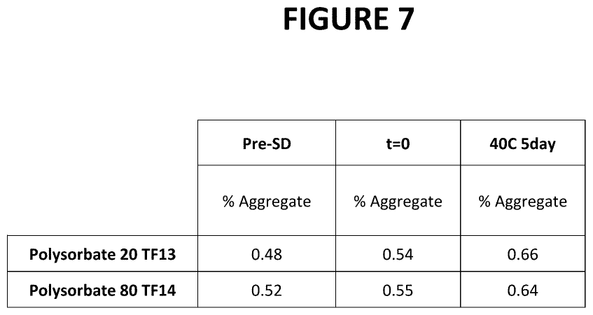

These formulations were then evaluated for level of protein aggregation in the pre-spray-dried aqueous formulation and in the solubilized spray-dried powder, via size exclusion chromatography, comparing samples tested pre-spray-drying and those pulled at Time 0 (e.g., within 1 day) after spray-drying and lyophilization, and others stored at 50° C. for one day. Results are shown in FIGS. 6-7. These results indicate that the polysorbate has a stabilizing effect upon spray-drying and post-spray-drying storage stability (i.e., reducing the aggregation of the powder). Under the conditions evaluated in this study, there did not appear to be a significant difference between polysorbate 20 and polysorbate 80 in stabilization of the powders. Moreover, only minor incremental gains in stability are seen with increasing amounts of polysorbate added to the pre-spray dry formulations; about 0.5 mg/mL of polysorbate 20 appears to provide a suitable improvement in stabilization without adding too much solids mass to the formulation (which would, as noted above for trehalose, negatively impact (dilute) the amount of active, in this case TmAb, that could be included in the paste formulation)

Next, the inclusion of various amino acids as excipients was evaluated for their effects upon aggregation and storage stability. First, various methionine-containing formulations were prepared according to the component amounts shown in Table 12:

TABLE 12
Methionine-containing Formulations

| | Feed | | Feed | | Feed | | Feed | |
|---|---|---|---|---|---|---|---|---|
| | Solution | Powder | Solution | Powder | Solution | Powder | Solution | Powder |
| | Batch | Batch | Batch | Batch | Batch | Batch | Batch | Batch |
| | TF30 | TF30 | TF33 | TF33 | TF34 | TF34 | TF35 | TF35 |
| TmAb | 20.0 | 76.63% | 20.0 | 72.46% | 20.0 | 68.73% | 20.0 | 62.31% |
| | mg/mL | | mg/mL | | mg/mL | | mg/mL | |
| TmAb | — | 565.4 | — | 534.6 | — | 507.1 | — | 459.7 |
| mg/mL | | mg/mL | | mg/mL | | mg/mL | | mg/mL |
| in paste | | | | | | | | |
| (assume | | | | | | | | |
| 60% w/w | | | | | | | | |
| load) | | | | | | | | |
| Trehalose | 5.15 | 19.73% | 5.15 | 18.66% | 5.15 | 17.70% | 5.15 | 16.04% |
| | mg/mL | | mg/mL | | mg/mL | | mg/mL | |
| Polysorbate | 0.5 | 1.92% | 0.5 | 1.81% | 0.5 | 1.72% | 0.5 | 1.56% |
| 20 | mg/mL | | mg/mL | | mg/mL | | mg/mL | |
| Methionine | — | — | 1.5 | 5.43% | 3 | 10.31% | 6 | 18.69% |
| | | | mg/mL | | mg/mL | | mg/mL | |
| Lactate | 0.450 | 1.72% | 0.450 | 1.63% | 0.450 | 1.55% | 0.450 | 1.40% |
| | mg/mL | | mg/mL | | mg/mL | | mg/mL | |
| | (5 mM) | | | | | | | |
| pH | 4.0 | — | 4.0 | — | 4.0 | — | 4.0 | — |
| Total | 26.10 | 100% | 27.60 | 100% | 29.10 | 100% | 32.10 | 100% |
| | mg/mL | | mg/mL | | mg/mL | | mg/mL | |

In a similar fashion, the inclusion of proline or glycine was evaluated in formulations prepared as shown in Tables 13 and 14:

TABLE 13
Proline/Glycine-containing Formulations

| | Feed | | Feed | | Feed | | Feed | |
|---|---|---|---|---|---|---|---|---|
| | Solution | Powder | Solution | Powder | Solution | Powder | Solution | Powder |
| | Batch | Batch | Batch | Batch | Batch | Batch | Batch | Batch |
| | TF30 | TF30 | TF40 | TF40 | TF41 | TF41 | TF42 | TF42 |
| TmAb | 20.0 | 76.63% | 20.0 | 55.40% | 20.0 | 55.40% | 20.0 | 43.38% |
| | mg/mL | | mg/mL | | mg/mL | | mg/mL | |
| TmAb | — | 565.4 | — | 408.8 | — | 408.8 | — | 320.1 |
| mg/mL | | mg/mL | | mg/mL | | mg/mL | | mg/mL |
| in paste | | | | | | | | |
| (assume | | | | | | | | |
| 60% w/w | | | | | | | | |
| load) | | | | | | | | |
| Trehalose | 5.15 | 19.73% | 5.15 | 14.27% | 5.15 | 14.27% | 5.15 | 11.17% |
| | mg/mL | | mg/mL | | mg/mL | | mg/mL | |
| Polysorbate | 0.5 | 1.92% | 0.5 | 1.39% | 0.5 | 1.39% | 0.5 | 1.08% |
| 20 | mg/mL | | mg/mL | | mg/mL | | mg/mL | |
| Proline | — | — | 10 | 27.70% | — | — | 10 | 21.69% |
| | | | mg/mL | | | | mg/mL | |
| Glycine | — | — | — | — | 10 | 27.70% | 10 | 21.69% |
| | | | | | mg/mL | | mg/mL | |
| Lactate | 0.450 | 1.72% | 0.450 | 1.25% | 0.450 | 1.25% | 0.450 | 0.98% |
| | mg/mL | | mg/mL | | mg/mL | | mg/mL | |
| pH | 4.0 | | 4.0 | | 4.0 | — | 4.0 | — |
| Total | 26.10 | 100% | 36.10 | 100% | 36.10 | 100% | 46.10 | 100% |
| | mg/mL | | mg/mL | | mg/mL | | mg/mL | |

TABLE 14
Proline-containing Formulations (continued)

| | Feed | | Feed | | Feed | |
|---|---|---|---|---|---|---|
| | Solution | Powder | Solution | Powder | Solution | Powder |
| | Batch | Batch | Batch | Batch | Batch | Batch |
| | TF30 | TF30 | TF49 | TF49 | TF50 | TF50 |
| TmAb | 20.0 | 76.63% | 20.0 | 62.31% | 20.0 | 55.40% |
| | mg/mL) | | mg/mL | | mg/mL | |
| TmAb | — | 565.4 | — | 459.7 | — | 408.8 |
| mg/mL | | mg/mL | | mg/mL | | mg/mL |
| in paste | | | | | | |
| (assume | | | | | | |
| 60% w/w | | | | | | |
| load) | | | | | | |
| Trehalose | 5.15 | 19.73% | 5.15 | 16.04% | 5.15 | 14.27% |
| | mg/mL | | mg/mL | | mg/mL | |
| Polysorbate | 0.5 | 1.92% | 0.5 | 1.56% | 0.5 | 1.39% |
| 20 | mg/mL | | mg/mL | | mg/mL | |
| Proline | — | — | 6 | 18.69% | 10 | 27.70% |
| | | | mg/mL | | mg/mL | |
| Lactate | 0.450 | 1.72% | 0.450 | 1.40% | 0.450 | 1.25% |
| | mg/mL | | mg/mL | | mg/mL | |
| pH | 4.0 | — | 4.0 | — | 4.0 | — |
| Total | 26.10 | 100% | 32.1 | 100% | 36.1 | 100% |
| | mg/mL | | mg/mL | | mg/mL | |

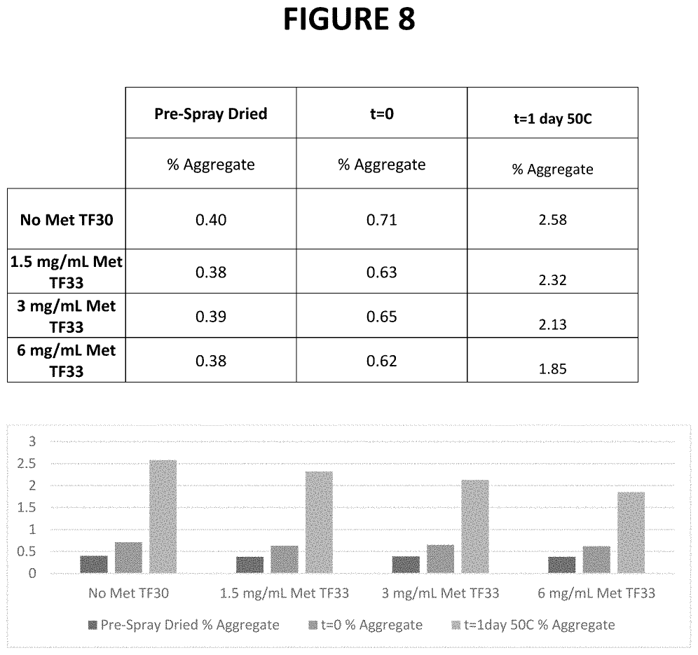

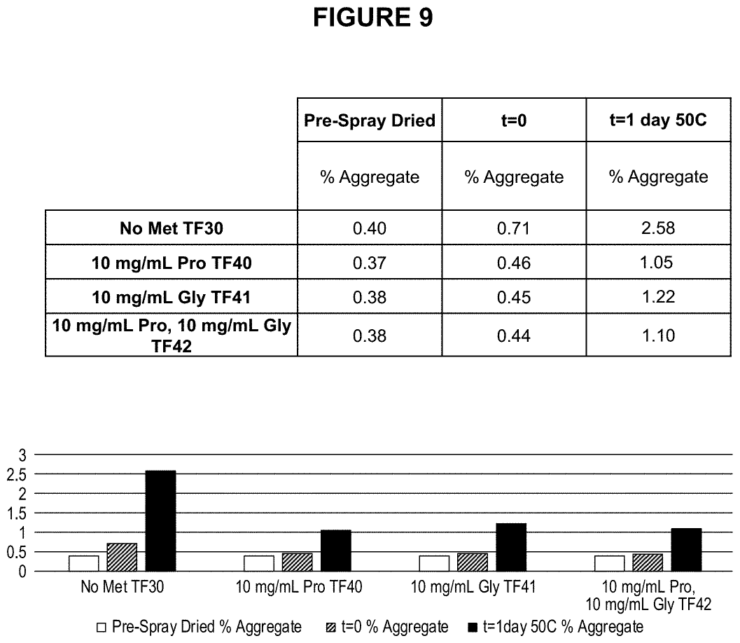

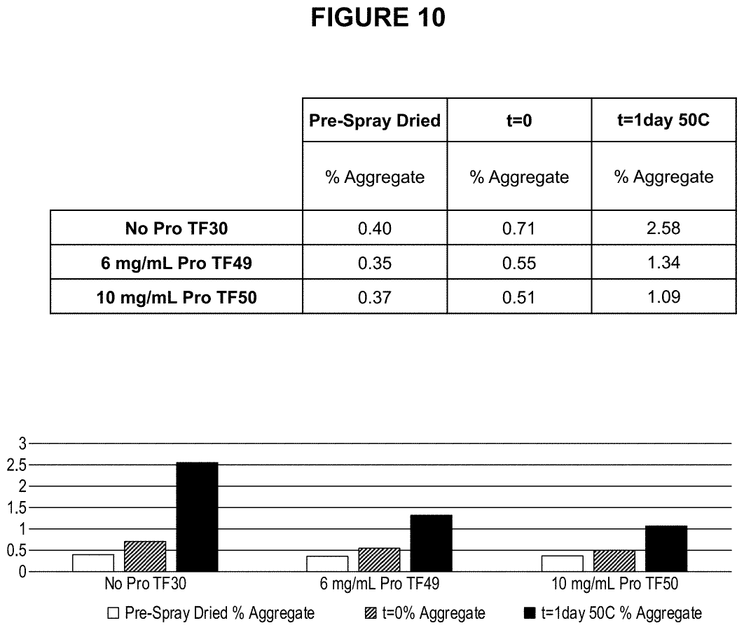

These formulations were then evaluated for level of aggregation in the pre-spray-dried formulation and in the solubilized spray-dried powder, via size exclusion chromatography, comparing samples tested pre-spray-drying and those samples after spray-drying and lyophilization (Time 0 (T0) samples), and others stored at 50° C. for one day. Results are shown in FIG. 8 (for methionine), FIG. 9 (for proline/glycine Round 1), and FIG. 10 (for proline Round 2). Taken together, these results indicate that while methionine (FIG. 8), proline (FIGS. 9 and 10) and glycine (FIG. 9) all act as stabilizing excipients, under the conditions evaluated in this study, relatively high amounts of these amino acids may be required to see more than just marginal impact upon stability. Since increased content of these excipients in the feed solution (while holding mAb content relatively constant) would dilute the active ingredient in the powder and resulting paste formulations, these amino acids may not be favored excipients for these formulations produced and evaluated under the conditions of this Example, as the reduction in active content in these formulations in exchange for only a minor increase in stability is not worth the trade-off.

Finally, formulations comprising cysteine at various concentrations were prepared according to Tables 15, 16, and 17:

TABLE 15
Cysteine-containing Formulations

| | Feed | | Feed | | Feed | |
|---|---|---|---|---|---|---|
| | Solution | Powder | Solution | Powder | Solution | Powder |
| | Batch | Batch | Batch | Batch | Batch | Batch |
| | TF36 | TF36 | TF37 | TF37 | TF38 | TF38 |
| TmAb | 20.0 | 72.46% | 20.0 | 55.40% | 20.0 | 48.66% |
| | mg/mL | | mg/mL | | mg/mL | |
| | (0.135 mM) | | (0.135 mM) | | (0.135 mM) | |
| TmAb | — | 534.6 | — | 408.8 | — | 359.0 |
| mg/mL | | mg/mL | | mg/mL | | mg/mL |
| in paste | | | | | | |
| (assume | | | | | | |
| 60% w/w | | | | | | |
| load) | | | | | | |
| Trehalose | 5.15 | 18.66% | 5.15 | 14.27% | 5.15 | 12.53% |
| | mg/mL | | mg/mL | | mg/mL | |
| | (15.0 mM) | | (15.0 mM) | | (15.0 mM) | |
| Polysorbate | 0.5 | 1.81% | 0.5 | 1.39% | 0.5 | 1.22% |
| 20 | mg/mL | | mg/mL | | mg/mL | |
| Cysteine | 1.5 | 5.43% | 10 | 27.70% | 15 | 36.50% |
| | mg/mL | | mg/mL | | mg/mL | |
| Lactate | 0.450 | 1.63% | 0.450 | 1.25% | 0.450 | 1.09% |
| | mg/mL | | mg/mL | | mg/mL | |
| | (5 mM) | | (5 mM) | | (5 mM) | |
| pH | 4.0 | — | 4.0 | — | 4.0 | — |
| Total | 27.60 | 100% | 36.10 | 100% | 41.10 | 100% |
| | mg/mL | | mg/mL | | mg/mL | |

TABLE 16
Cysteine-containing Formulations (continued)

| | Feed | | Feed | | Feed | | Feed | |
|---|---|---|---|---|---|---|---|---|
| | Solution | Powder | Solution | Powder | Solution | Powder | Solution | Powder |
| | Batch | Batch | Batch | Batch | Batch | Batch | Batch | Batch |
| | TF43 | TF43 | TF44 | TF44 | TF45 | TF45 | TF46 | TF46 |
| TmAb | 20.0 | 72.46% | 20.0 | 68.73% | 20.0 | 62.31% | 20.0 | 55.40% |
| | mg/mL | | mg/mL | | mg/mL | | mg/mL | |
| TmAb | — | 534.6 | — | 507.1 | — | 459.7 | — | 408.8 |
| mg/mL | | mg/mL | | mg/mL | | mg/mL | | mg/mL |
| in paste | | | | | | | | |
| (assume | | | | | | | | |
| 60% w/w | | | | | | | | |
| load) | | | | | | | | |
| Trehalose | 5.15 | 18.66% | 5.15 | 17.70% | 5.15 | 16.04% | 5.15 | 14.27% |
| | mg/mL | | mg/mL | | mg/mL | | mg/mL | |
| Polysorbate | 0.5 | 1.81% | 0.5 | 1.72% | 0.5 | 1.56% | 0.5 | 1.39% |
| 20 | mg/mL | | mg/mL | | mg/mL | | mg/mL | |
| Cysteine | 1.5 | 5.43% | 3 | 10.31% | 6 | 18.69% | 10 | 27.70% |
| | mg/mL | | mg/mL | | mg/mL | | mg/mL | |
| Lactate | 0.450 | 1.63% | 0.450 | 1.55% | 0.450 | 1.40% | 0.450 | 1.25% |
| | mg/mL | | mg/mL | | mg/mL | | mg/mL | |
| pH | 4.0 | — | 4.0 | — | 4.0 | | 4.0 | — |
| Total | 27.6 | 100% | 29.1 | 100% | 32.1 | 100% | 36.1 | 100% |
| | mg/mL | | mg/mL | | mg/mL | | mg/mL | |

TABLE 17
Cysteine-containing Formulations (continued)

| | Feed Solution | Powder Batch | Feed Solution | Powder Batch |
|---|---|---|---|---|
| | Batch TF47 | TF47 | Batch TF48 | TF48 |
| TmAb | 20.0 mg/mL | 61.68% | 20.0 mg/mL | 54.91% |
| | (0.135 mM) | | (0.135 mM) | |
| TmAb mg/mL in | — | 455.1 mg/mL | — | 405.1 mg/mL |
| paste (assume | | | | |
| 60% w/w load) | | | | |
| Trehalose | 5.15 mg/mL | 15.88% | 5.15 mg/mL | 14.14% |
| | (15.0 mM) | | (15.0 mM) | |
| Polysorbate 20 | 0.5 mg/mL | 1.54% | 0.5 mg/mL | 1.37% |
| Cysteine | 6 mg/mL | 18.50% | 10 mg/mL | 27.45% |
| Proline | — | — | — | — |
| Lactate | — | — | — | — |
| Histidine | 0.774 mg/mL | 2.39% | 0.774 mg/mL | 2.12% |
| | (5 mM) | | (5 mM) | |
| pH | 6.0 | — | 6.0 | — |
| Total | 32.42 mg/mL | 100% | 36.42 mg/mL | 100% |

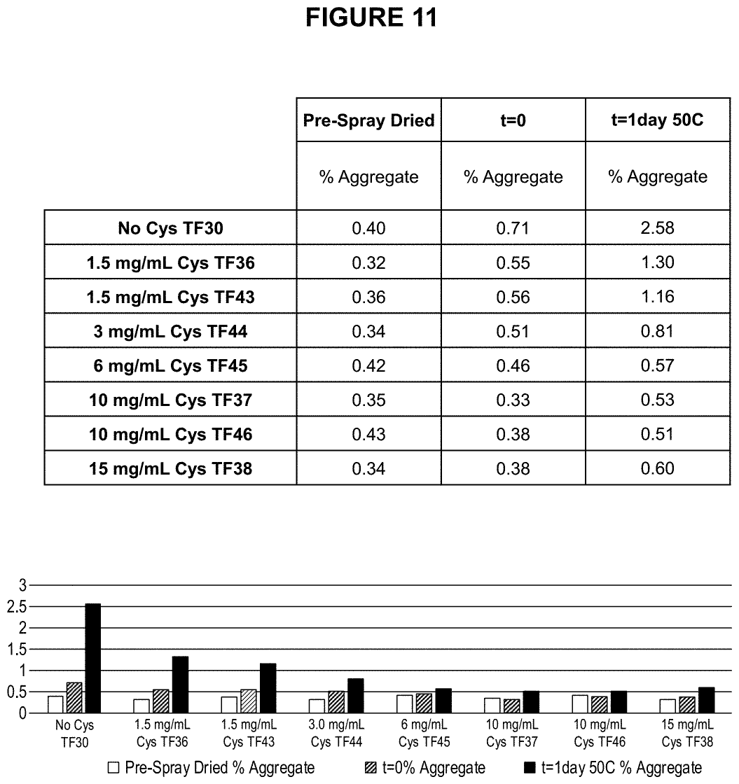

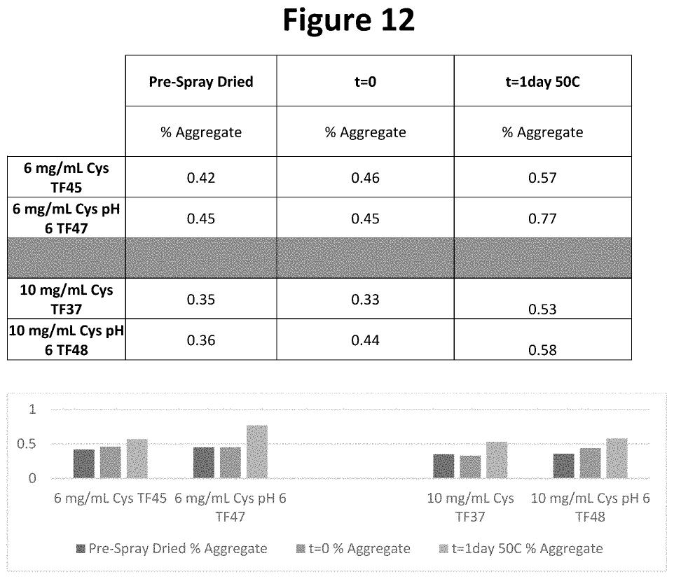

These formulations were then evaluated for mAb aggregation in the pre-spray-dried formulation and in the solubilized spray-dried powder, via size exclusion chromatography, comparing samples tested pre-spray-drying and those pulled at t=0 after spray-drying and lyophilization, and others stored at 50° C. for one day. Results are shown in FIGS. 11-12, and suggest that including cysteine even at low amounts in the pre-spray-dried formulation may have a strongly positive effect on storage stability of the spray-dried powders prepared under the conditions described in this example.

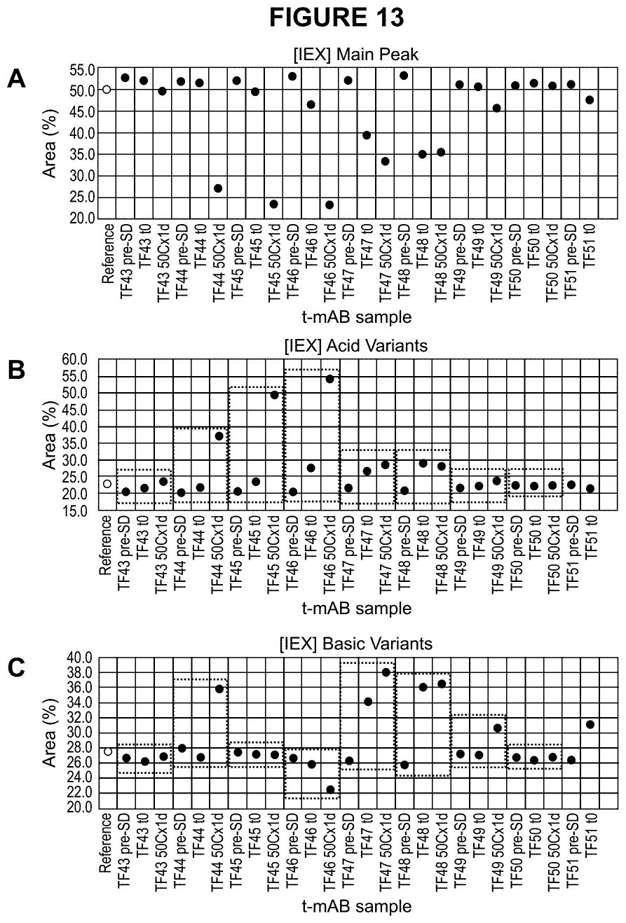

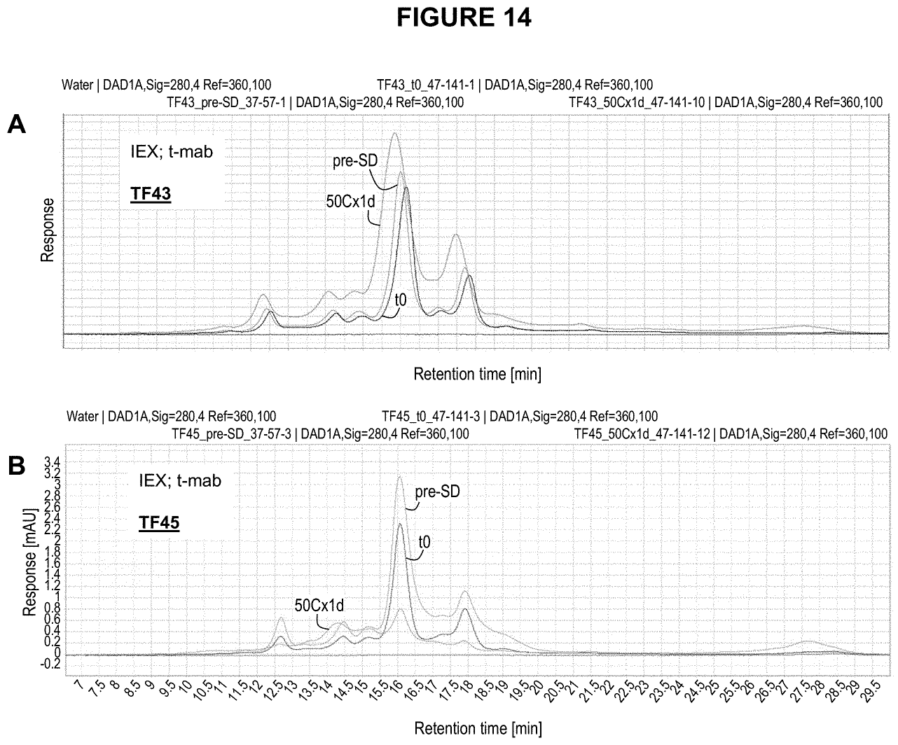

To further evaluate the effects of cysteine, powders prepared from various cysteine-containing formulations were dissolved and then analyzed by ion exchange chromatography. These results are shown in FIGS. 13 and 14; FIG. 13 shows the levels (in terms of percentage of total, indicated by AUC measurement) of the main peak (FIG. 13A), acidic variants (FIG. 13B) and basic variants (FIG. 13C) present in cysteine-containing formulations of TmAb, while FIG. 14 provides representative trace of the stability profiles of the two formulations, one containing 1.5 mg/mL cysteine (FIG. 14A) and the other containing 6 mg/mL cysteine (FIG. 14B). Taken together, these results indicate that high levels of cysteine may disrupt the internal Cys-Cys bonds that are present in the TmAb molecule, possibly leading to inactivation of the antibody and therefore loss of its therapeutic effects in addition to a loss in storage stability. Thus, inclusion of a lower cysteine content (e.g., 1.5 mg/mL) in the pre-spray-drying formulation may enhance stability while avoiding the loss of storage stability and potential loss of bioactivity brought on by inclusion of higher amounts of cysteine in the formulations.

Finally, dialyzed solutions of TmAb were prepared in an optimized excipient formulation and used to optimize certain spray-drying parameters, in particular the optimum feed solution concentration and inlet temperature settings for maximizing protein concentration and storage stability while minimizing aggregation. Formulations were prepared as shown in Table 18:

TABLE 18
Formulations for Optimizing Spray-Drying Parameters

| | Feed | | Feed | | Feed | |
|---|---|---|---|---|---|---|
| | Solution | | Solution | | Solution | |
| | Batch | Powder | Batch | Powder | Batch | Powder |
| | TF29a | Batch | TF29b | Batch | TF29c | Batch |
| | (70° C. inlet) | TF29a | (80° C. inlet) | TF29b | (90° C. inlet) | TF29c |
| TmAb | 20.0 | 76.63% | 20.0 | 76.63% | 20.0 | 76.63% |
| | mg/mL | | mg/mL | | mg/mL | |
| | (0.135 mM) | | (0.135 mM) | | (0.135 mM) | |
| TmAb | — | 565.4 | — | 565.4 | — | 565.4 |
| mg/mL | | mg/mL | | mg/mL | | mg/mL |
| in paste | | | | | | |
| (assume | | | | | | |
| 60% w/w | | | | | | |
| load) | | | | | | |
| Trehalose | 5.15 | 19.73% | 5.15 | 19.73% | 5.15 | 19.73% |
| | mg/mL | | mg/mL | | mg/mL | |
| | (15.0 mM) | | (15.0 mM) | | (15.0 mM) | |
| Polysorbate | 0.5 | 1.92% | 0.5 | 1.92% | 0.5 | 1.92% |
| 20 | mg/mL | | mg/mL | | mg/mL | |
| Lactate | 0.450 | 1.72% | 0.450 | 1.72% | 0.450 | 1.72% |
| | mg/mL | | mg/mL | | mg/mL | |
| | (5 mM) | | (5 mM) | | (5 mM) | |
| pH | 4.0 | — | 4.0 | — | 4.0 | — |
| Total | 25.1 | 100% | 25.1 | 100% | 25.1 | 100% |
| | mg/mL | | mg/mL | | mg/mL | |
| | Feed | | Feed | | Feed | |
| | Solution | | Solution | | Solution | |
| | Batch | Powder | Batch | Powder | Batch | Powder |
| | TF52a | Batch | TF52b | Batch | TF52c | Batch |
| | (70° C. inlet) | TF52a | (80° C. inlet) | TF52b | (90° C. inlet) | TF52c |
| TmAb | 30.0 | 76.63% | 30.0 | 76.63% | 30.0 | 76.63% |
| | mg/mL | | mg/mL | | mg/mL | |
| | (0.202 mM) | | (0.202 mM) | | (0.202 mM) | |
| TmAb | — | 565.4 | — | 565.4 | — | 565.4 |
| mg/mL | | mg/mL | | mg/mL | | mg/mL |
| in paste | | | | | | |
| (assume | | | | | | |
| 60% w/w | | | | | | |
| load) | | | | | | |
| Trehalose | 7.72 | 19.73% | 7.72 | 19.73% | 7.72 | 19.73% |
| | mg/mL | | mg/mL | | mg/mL | |
| | (22.5 mM) | | (22.5 mM) | | (22.5 mM) | |
| Polysorbate | 0.75 | 1.92% | 0.75 | 1.92% | 0.75 | 1.92% |
| 20 | mg/mL | | mg/mL | | mg/mL | |
| Lactate | 0.675 | 1.72% | 0.675 | 1.72% | 0.675 | 1.72% |
| | mg/mL | | mg/mL | | mg/mL | |
| | (7.5 mM) | | (7.5 mM) | | (7.5 mM) | |
| pH | 4.0 | — | 4.0 | — | 4.0 | — |
| Total | 37.65 | 100% | 37.65 | 100% | 37.65 | 100% |
| | mg/mL | | mg/mL | | mg/mL | |

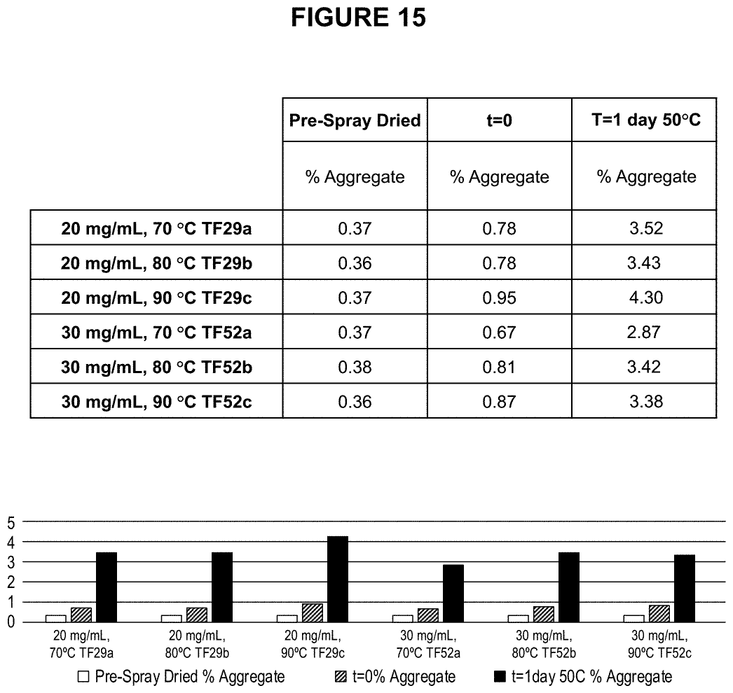

Additional formulations were evaluated by ion exchange chromatography for aggregation both pre-spray drying and at t=0 and t=1 day at 50° C. post-spray-drying. Results are shown in FIG. 15. These results demonstrate that use of a higher feed solution concentration (e.g., 30 mg/mL TmAb vs. 20 mg/mL TmAb) results in the production of less aggregated starting solutions, less aggregated spray-dried powders and more storage-stable powders at a given spray dryer inlet temperature (compare, e.g., results in FIG. 15 for Batch 52a vs. Batch 29a; Batch 52b vs. Batch 29b; and Batch 52c vs. Batch 29c). In addition, at a given feed solution concentration, the 70° C. inlet temperature appeared to result in the production of less aggregated (at t=0) and more storage stable (at t=1 day at 50° C.) powders, vs. those prepared at higher inlet temperatures (compare, e.g., results in FIG. 15 for Batch 29a vs. Batches 29b and 29c; and those for Batch 52a vs. Batches 52b and 52c); this result confirms those reported above in Example 1 with respect to the optimum inlet temperature.

Based on the studies described above, the composition detailed in Table 19 represents one example formulation exhibiting good antibody stability and while also providing for high antibody drug concentration (>400 mg/mL) in the resulting paste formulations:

TABLE 19
Xeriject ™ Paste Formulation for
a High Concentration TmAb Paste

| | Feed Solution | Powder Batch |
|---|---|---|
| Antibody (e.g., TmAb) conc. | 20.0 mg/mL | 62.31% |
| | (0.135 mM) | |
| Antibody mg/mL in paste | — | 459.7 mg/mL |
| (assume 60% w/w load) | | |
| Trehalose | 5.15 mg/mL | 16.04% |
| | (15.0 mM) | |
| Polysorbate 20 | 0.5 mg/mL | 1.56% |
| Proline | 6 mg/mL | 18.69% |
| Lactate | 0.450 mg/mL | 1.40% |
| | (5 mM) | |
| pH | 4.0 | — |
| Total | 32.1 mg/mL | 100% |

Altogether, these studies provide examples of representative aqueous feed solution formulations and spray-drying and process conditions to produce highly-concentrated, high solids content, and storage-stable dry powder preparations of therapeutic proteins such as monoclonal antibodies, which are suitable for use in preparing the high concentration injectable paste formulations that permit the intracutaneous, subcutaneous and/or intramuscular injection of therapeutic peptides and proteins that previously could only be administered intravenously over considerably longer time periods. In fact, using the approach described in this Example, it is possible to prepare a matrix of formulation and apparatus parameters that would facilitate excipient and process parameter screening to promote the development of suitable formulations for the preparation of high solids concentration formulations that may be advantageously spray-dried into flowable, storage-stable paste formulations for therapeutic uses. This approach—preparing high solids concentrations injectable paste therapeutic formulations, a technology developed by Xeris Pharmaceuticals, Inc. under its Xeriject™ technology platform that is described herein—therefore provides a number of patient benefits, including convenience of dosing, avoidance of discomfort and possibly efficacy of the therapeutic peptide/protein formulation.

### Example 3

### Production of High Concentration Pastes Comprising Therapeutic Monoclonal Antibodies

XeriJect™ (XJ) is a proprietary formulation technology that can significantly increase the concentration and/or the thermostability of an active pharmaceutical ingredient (API) in a dose. With the XeriJect™ technology, dry particles of an active pharmaceutical ingredient (API), preferably prepared by spray-drying according to the methods described in Examples 1 and 2 above, are blended with a non-solvent liquid and mixed to form a paste. As described previously, a paste is a two-phase composition residing on the spectrum between suspension and wetted solid, wherein the solids concentration in the powder, With this approach, drug concentrations of 30% w/w or higher can be achieved (250 mg/mL or higher). This technology represents a significant improvement over current therapies, which often must be administered as a long-duration IV infusion of a low-concentration solution in a clinic. This technology can be used to deliver high doses of proteins, such as antibodies, or small molecules to a patient with a bolus subcutaneous dose. In addition, in certain formulations the Xeriject™ technology can provide increased thermostability of the formulation (particularly the active pharmaceutical ingredient(s) in the formulation) even at standard lower-dose concentrations.

In these studies, the Xeriject™ technology was evaluated as a platform for the small volume subcutaneous delivery of therapeutics that previously were only capable of being administered intravenously. Specifically, several monoclonal antibody products that are commercially available were formulated into Xeriject™ paste formulations:

TABLE 20
Commercial Drug Products Prepared in Paste Form Using Xeriject ™ Technology

| | | | | | | | 400 mg/mL |
|---|---|---|---|---|---|---|---|
| | | Delivered | | Delivery | Current | | Paste |
| Product | Indication | Dose | | Route | Volume | | Formulation |
| OPDIVO ® | Non-small Cell Lung | 240 | mg | I.V. | 250 | mL | 0.60 mL |
| | Cancer | | | | | | |
| HUMIRA ® | Crohn's Disease | 160 | mg | S.C. | 3.2 | mL | 0.40 mL |
| HUMIRA ® | Rheumatoid Arthritis | 40 | mg | S.C. | 0.8 | mL | 0.10 mL |
| XGEVA ® | Skeletal Events (from bone | 120 | mg | S.C. | 1.7 | mL | 0.30 mL |
| | metastases) | | | | | | |
| ENTYVIO ® | Inflammatory Bowel | 300 | mg | I.V. | 250 | mL | 0.75 mL |
| | Disease | | | | | | |
| REMICADE ® | Crohn's Disease | 350 | mg¹ | I.V. | 250 | mL | 0.88 mL |
| REMICADE ® | Rheumatoid Arthritis | 210 | mg² | I.V. | 250 | mL | 0.53 mL |
| TYSABRI ® | Multiple Sclerosis | 300 | mg | I.V. | 115 | mL | 0.75 mL |
| KADCYLA ® | Metastatic Breast Cancer | 252 | mg³ | I.V. | 250 | mL | 0.63 mL |
| YERVOY ® | Metastatic Melanoma | 210 | mg⁴ | I.V. | 100 | mL | 0.53 mL |

¹5 mg/kg, 70 kg patient
²3 mg/kg, 70 kg patient
³3.6 mg/kg, 70 kg patient
⁴3 mg/kg, 70 kg patient

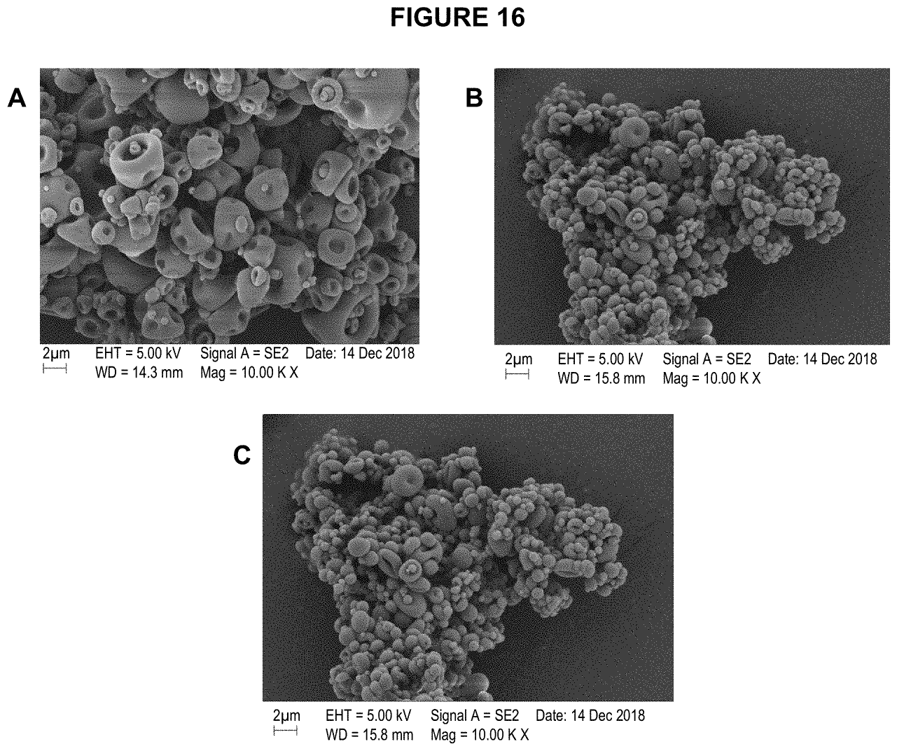

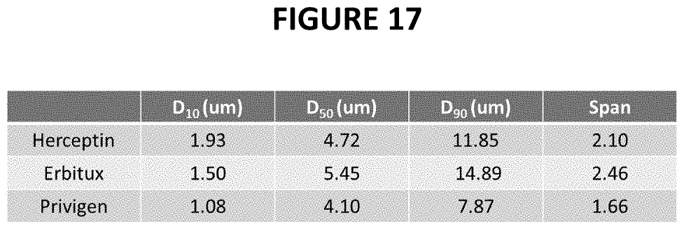

To set a baseline, samples of three other commercial products—Herceptin® (TmAb; see Examples 1-2), Erbitux® (cetuximab; Eli Lilly and Company), and Privigen® (immune globulin; CSL Behring AG)—were spray-dried from the commercial formulations with minimal modification, and then examined by scanning electron microscopy to observe the morphology and size of the antibody particles in the powdered products. Representative photomicrographs are shown in FIG. 16, which shows particles observed in spray-dried formulations of TmAb (FIG. 16A), cetuximab (FIG. 16B) and immune globulin (FIG. 16C) which are reminiscent of those observed for other therapeutic proteins in Example 1. Most of these commercial products demonstrated a broad range of particle sizes, and the TmAb commercial formulation also showed a high degree of toroidal particles which, based on the results presented in Example 1, are known to result in less than optimal powder materials for use in preparing therapeutic pastes using the Xeriject™ formulation technology. In fact, when the particle size distribution for these three formulations was obtained by laser diffraction, there was a polydisperse character as characterized by the range observed for the 10^{th} percentile to the 90^{th} percentile of particle diameters in all of the formulations, as shown in FIG. 17.

To prepare Xeriject™ paste formulations of these commercial products, small batches of each were spray-dried and pastes prepared according to the methods described above in Examples 1 and 2. Sucrose instead of trehalose was added to the commercial cetuximab and immune globulin formulations, and the cetuximab salt content was reduced by dialysis. The TmAb formulation was spray-dried from the commercial formulation. Pastes were prepared from the spray-dried powders and triacetin (density=1.16 g/mL) was included such that the final estimated paste density was about 1.24 g/mL. The total solids and active ingredient contents in each paste formulation were then calculated to be at the values shown in Table 21:

TABLE 21
Solids and Active Ingredient Content of Xeriject ™ mAb Pastes

| | | | mg Solids | | mg mAb |
|---|---|---|---|---|---|
| | Residual | Percent | Per mL | % mAb in | Per mL |
| | Moisture | Solids | Paste | Formulation | Paste |
| Herceptin | 0.88% | 45.2% | 560.5 mg | 51.3% | 287.5 mg |
| Erbitux | 2.29% | 42.0% | 520.8 mg | 66.5% | 346.3 mg |
| Privigen | 1.38% | 42.0% | 520.8 mg | 60.0% | 312.5 mg |

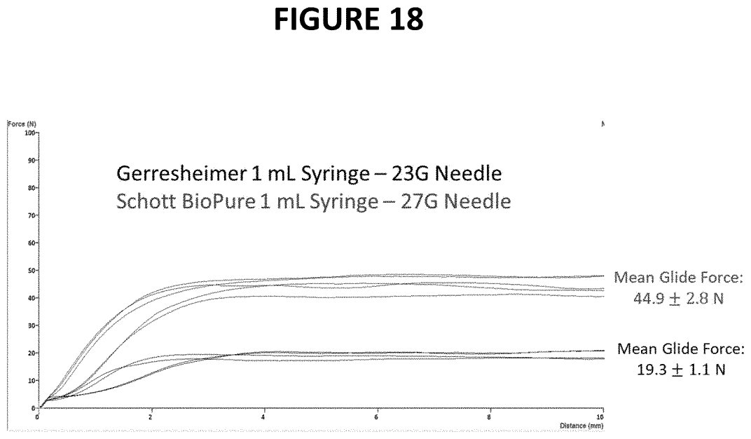

Following paste preparation, the formulations were evaluated for the injection force necessary to deliver 1 mL of a given paste in two different syringe/needle combinations: a 1 mL syringe with a staked 23 gauge ½ inch regular wall (RW) needle (Gerresheimer AG; Bunde, Germany) and a syriQ BioPure® 1 mL long syringe with a 27 gauge ½ inch regular wall (RW) needle (Schott AG; Mainz, Germany). For each configuration, a plunger velocity of 3.0 mm/sec (corresponding to a volumetric flow rate of approximately 98 uL/sec) was used to generate the injection force profile. Representative results with a preparation of Privigen® (immune globulin) prepared with triacetin as the diluent (continuous phase) at a 42% solids content are shown in FIG. 18. As expected, a significantly lower injection force was necessary to deliver the 1 mL paste in the larger (lower gauge) needle; this result confirms prior results using lower concentration paste formulations. Importantly, however, the full 1 mL of the paste product was delivered from even the 27 G needle without the need for overly excessive injection force. The ability to intracutaneously inject paste formulations of therapeutic antibodies in low volumes using relatively small gauge needles appropriate for intracutaneous and/or intramuscular injection and having relatively low injection forces significantly enhances the patient benefits of such injections, in terms of decreasing discomfort and the time required to receive a therapeutic antibody treatment, compared to standard IV administration of such antibodies.

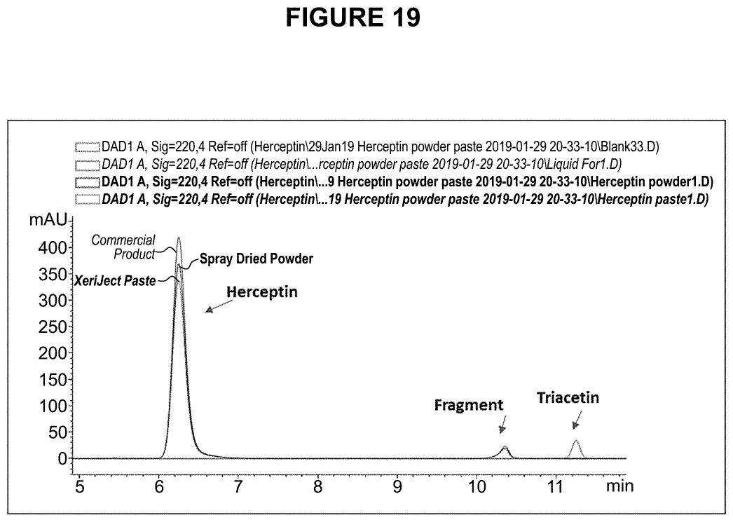

Next, the ability to formulate TmAb into a usable high-concentration paste was evaluated. The commercial product was reconstituted in water, and samples were held in solution, spray-dried into a powder, or formulated from the spray-dried powder into a paste (45.2% solids in triacetin) using the Xeriject™ technology described above. Evaluation of these samples by size exclusion chromatography demonstrated very little difference between them (FIG. 19)—all of the samples contained both a main peak and a fragment with identical retention profiles, while the Xeriject™ paste sample contained an additional peak corresponding to the triacetin in the paste formulation. Thus, the spray-drying and paste formation processes did not result in the aggregation of the TmAb protein when compared to reconstituted commercial product in solution.

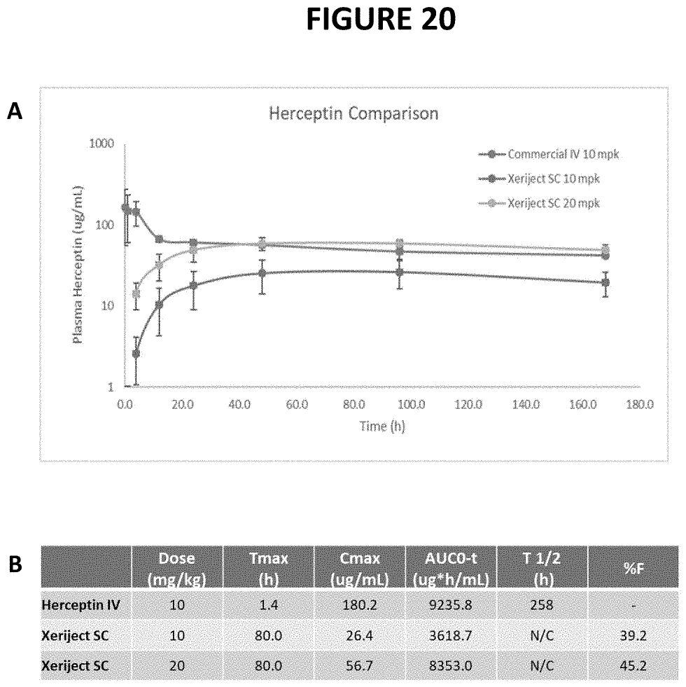

To evaluate the pharmacokinetics of these various TmAb formulations, and specifically those of the Xeriject™ paste formulations, the commercial antibody solution was spray-dried as described in the preceding examples, producing a spray-dried powder that was used to prepare a high solids content paste as described above. The API loading in this formulation was 259 mg/mL, and it was dosed by subcutaneous injection to male Sprague Dawley rats at two different dose volumes, using commercially available syringe/needle combinations. A commercial formulation of trastuzumab was also dosed by IV into control animals for comparison. As seen in FIG. 20, the IV dose of trastuzumab showed an early spike in drug level, where the XeriJect™ formulations did not have a spike and rose to a plateau drug level after ˜24 hours (FIG. 20A). Over the next six days of sampling, the XeriJect™ formulation and the IV dose maintained their plateau drug levels. As depicted in FIG. 20B, the pharmacokinetics for the XeriJect™ formulation were dose-dependent, with the 20 mg/kg XeriJect™ plateau drug level being similar to that of the 10 mg/kg IV dose. The Cₘₐₓ was blunted by about 15% for both the 10 mg/kg and 20 mg/kg doses of Xeriject™ TmAb compared with IV TmAb, while the bioavailability (AUCo-t) of the 10 mg/kg and 20 mg/kg doses of Xeriject™ TmAb compared with 10 mg/kg IV TmAb was 39 and 45%, respectively (dose normalized). Finally, while the T½ of the IV TmAb was about 10 days, the T½ for the Xeriject™ formulations was longer than the time allotted for the study and was not determined here. As those of ordinary skill in the pharmaceutical and medical arts will readily understand, a blunted Cₘₐₓ and sustained exposure (i.e., longer T^{1/2}) can be favorable for compounds that have Cₘₐₓ-driven toxicity profiles and AUC-driven efficacy, thereby providing another benefit of the pastes prepared using the Xeriject™ technology of the present invention. These results indicate that a high amount of drug can be administered in a relatively low volume in a bolus injection, while achieving therapeutically beneficial circulating drug levels with similar kinetics (albeit longer-lasting) as are achieved with IV administration. The Xeriject™ approach thus significantly improves this particular drug treatment/delivery and may be similarly useful for other treatments using high molecular weight peptides and proteins such as therapeutic antibodies and enzymes.

### Example 4

### Production of High Solids Pastes Comprising Therapeutic Enzymes

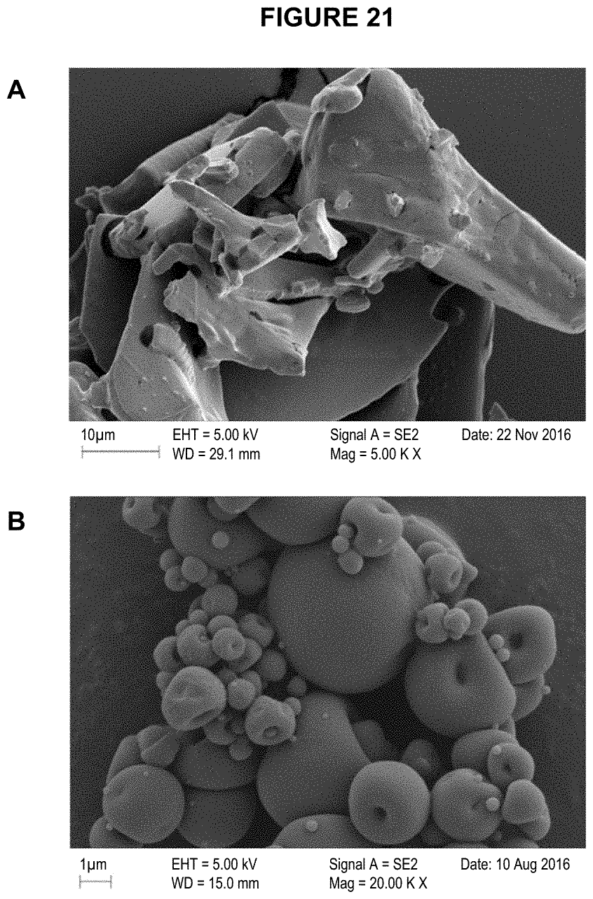

To further examine the utility of the Xeriject™ technology provided by the present invention, high solid concentration pastes comprising therapeutic enzymes were prepared. In these exemplary studies, a multimeric PEGylated therapeutic enzyme was used as the active ingredient. In a first step, solutions of the PEGylated enzyme were converted into dry powders by lyophilization and by the spray-drying processes described in the preceding examples. Samples of each powder preparation were then examined by scanning electron microscopy for morphology and size distribution. Representative photomicrographs are shown in FIG. 21. As seen in FIG. 21A, the PEGylated enzyme powder prepared by lyophilization of the feed solution demonstrated large, irregular particles with a relatively high specific surface area. In contrast, as seen in FIG. 21B, the powder prepared by the spray-drying processes described herein demonstrated small, spherical particles with a comparatively lower specific surface area. Moreover, when pastes were prepared from both of these powders, the pastes prepared from spray-dried powders exhibited a higher solids concentration, and thus a higher enzyme concentration, than pastes prepared from the lyophilized powders, as shown in Table 22:

TABLE 22
Characteristics of Enzyme Pastes

| | % Solids | Paste | Solids | Enzyme (% w/w) | Approx. Enzyme |
|---|---|---|---|---|---|
| Powder | Content | Density | Concentration | In Powder | Concentration |
| Lyophilized | 36.9% | 1.24 g/mL | 457.6 mg/mL | 16.8% | 77.0 mg/mL |
| Spray-Dried | 43.0% | 1.26 g/mL | 544.9 mg/mL | 16.8% | 91.7 mg/mL |

These results correspond with the discussion in the foregoing examples herein, that powders that comprise more regular, spherical particles of smaller size and surface area distribution are more suitable for the production of flowable pastes of active ingredients, and ultimately are more suitable for therapeutic subcutaneous injection into animals. Therefore, the remainder of these studies were conducted using spray-dried powders as starting materials for the production of the therapeutic Xeriject™ pastes.

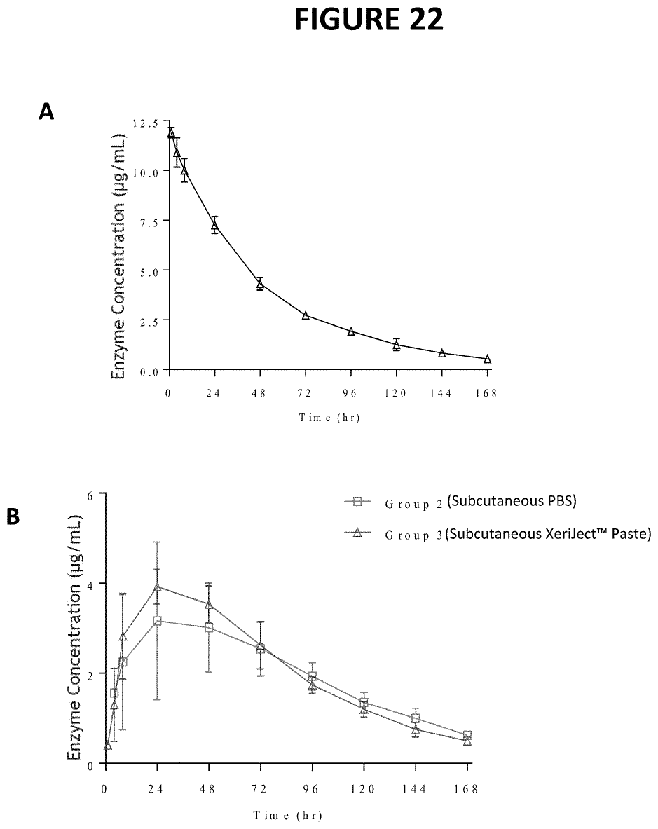

To study the pharmacokinetic parameters of the Xeriject™ enzyme paste formulations, the spray-dried paste enzyme preparations described above were dosed by subcutaneous injection to male Sprague Dawley rats, using commercially available syringe/needle combinations. An aqueous formulation of the enzyme (in PBS) was also dosed by IV and subcutaneously into control animals for comparison. Plasma enzyme concentrations in each group of animals were then determined over time, with results shown in FIG. 22. The aqueous (PBS) formulation administered intravenously demonstrated an initial spike of enzyme in the plasma followed by a rapid decline over the next 48-72 hours (FIG. 22A). In contrast, the subcutaneous aqueous (PBS) and Xeriject™ paste formulations demonstrated similar pharmacokinetic profiles with a slower rise to peak plasma concentration followed by a more prolonged and gradual decrease (FIG. 22B). As shown in Table 23, the T½, Tₘₐₓ, Cₘₐₓ and AUC(0−t) values of the subcutaneously administered formulations were also similar and significantly different from those for the IV-administered enzyme, reminiscent of results obtained with IV aqueous vs. subcutaneous Xeriject™ paste formulations of TmAb described in Example 2 herein:

TABLE 23
Pharmacokinetic Profiles of IV and
Subcutaneous Enzyme Formulations

| | T½ | Tmax | Cmax | AUC(0-t) |
|---|---|---|---|---|
| Group | (hr) | (hr) | (μg/mL) | (hr*μg/mL) |
| Intravenous | 37.0 ± 1.8 | 1.0 ± 1.0 | 11.9 ± 0.3 | 576.0 ± 19.1 |
| PBS | | | | |
| Subcutaneous | 46.7 ± 3.1 | 24.0 ± 24.0 | 3.38 ± 1.4 | 331.0 ± 106 |
| PBS | | | | |
| Subcutaneous | 38.1 ± 1.6 | 24.0 ± 24.0 | 3.92 ± 0.4 | 351.0 ± 53.4 |
| XeriJect ™ | | | | |

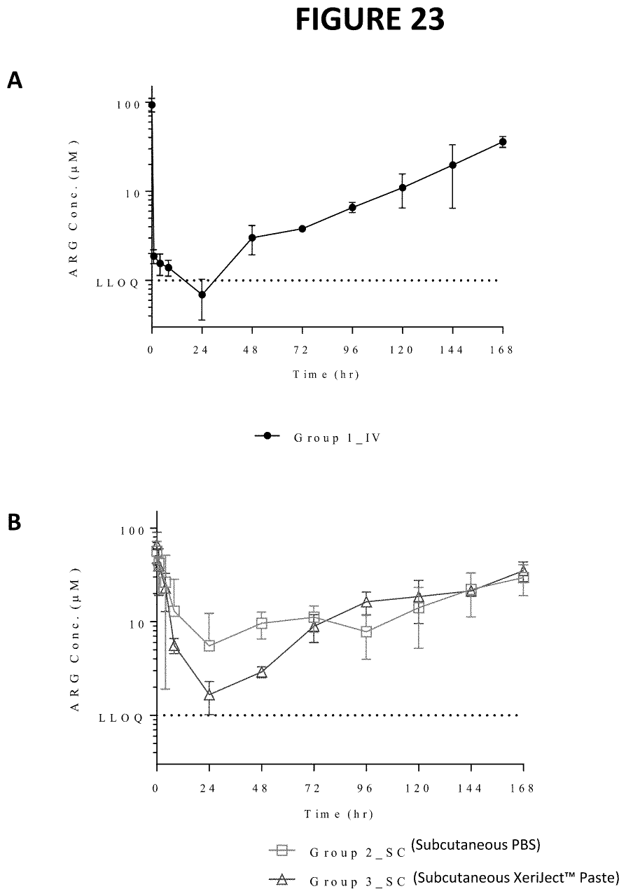

Pharmacodynamic analysis of plasma samples from the treated animals demonstrated a difference not only between IV-administered (FIG. 23A) and subcutaneous-administered (FIG. 23B) enzyme formulations, but also between subcutaneously administered aqueous (PBS) and Xeriject™ paste formulations (FIG. 23B). Specifically, while the aqueous and paste formulations exhibited similar pharmacokinetic profiles as described above, the Xeriject™ paste formulation showed a greater peak target depletion in pharmacodynamic studies (6 μM vs. 2 Moreover, the results of a post-sacrifice pathology report (not shown) indicated that there were no significant injection site reactions (and thus no post-injection discomfort) observed in animals that had been injected subcutaneously with the Xeriject™ paste formulations.

Taken together, these results indicate that the paste formulations of high concentration high molecular weight protein therapeutics that are provided by the present invention are useful in delivering controlled- or sustained-release depots of a therapeutic protein in small volumes subcutaneously in a way that improves the patient experience from that obtained with traditional intravenous administration of such therapeutic proteins.

### Example 5

### Production of High Solids Pastes Comprising High Concentration Glucagon

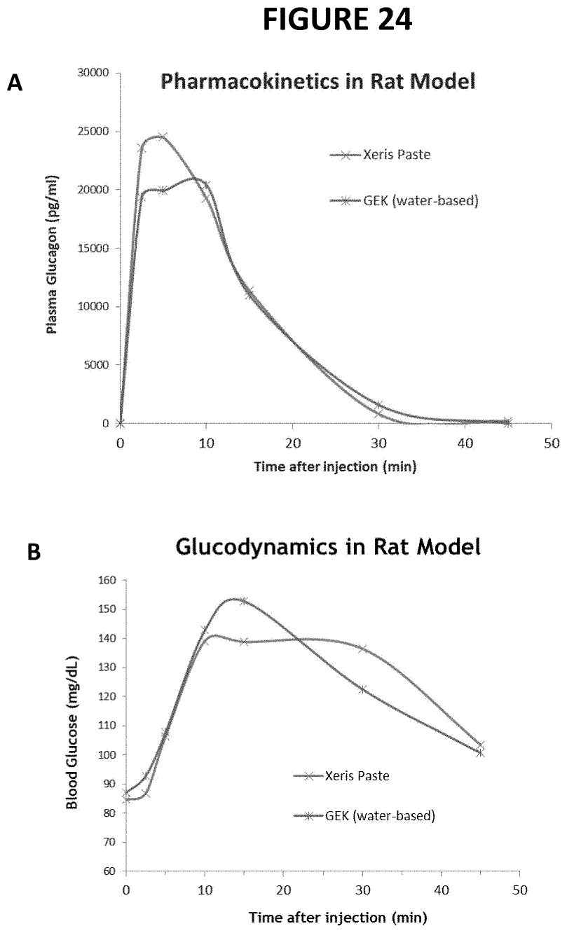

In additional studies, Xeriject™ paste formulations containing high-concentration glucagon were prepared by thin-film-freezing (a particle engineering technology producing powders having relatively high surface area) from an aqueous solution containing glucagon, trehalose, and a buffering agent (glycine) and adjusted to pH 3.0. The thin-film freeze-dried powder was formulated into a paste by mildly grinding and blending with sufficient triacetin create a paste. 1 mg of glucagon was administered as 5 ul of the paste subcutaneously to rats at the same dose as a larger volume (1 ml) of commercially available aqueous glucagon formulation (glucagon emergency kit or “GEK”; Eli Lilly). This allowed for a direct comparison of the pharmacokinetics and pharmacodynamics of the Xeriject™ paste formulation to a commercial aqueous solution. Results are shown in FIG. 24.

Examination of the pharmacokinetic profiles of the two formulations (FIG. 24A) indicated that despite being injected in a 200× lower volume, the Xeriject™ (Xeris) paste formulations exhibited comparable pharmacokinetics as the aqueous formulations of glucagon injected in a higher volume. The same was true of the pharmacodynamic profiles (FIG. 24B), with the Xeriject™ (Xeris) paste formulations showing similar pharmacodynamics as observed for the aqueous GEK formulations, albeit a slower decay over time as has been seen with other therapeutic peptides (see preceding Examples). These results indicate that the paste formulations of a higher concentration peptide (glucagon) that are provided by the present invention are useful in delivering of glucagon in small volumes subcutaneously in a way that improves the patient experience from that obtained with traditional intramuscular administration of aqueous formulations of glucagon that require relatively larger volume injections.

### Example 6

### Production of High Solids Pastes Comprising Insulin

In addition to enabling injectable formulations having very high concentrations of therapeutic agents, injectable pastes described herein may also be utilized to significantly enhance the thermostability of a therapeutic agent, including those administered at relatively lower concentrations. One such example is human insulin, which is commercially available in concentrations ranging from u100 (˜3.5 mg/mL) to u500 (˜17.4 mg/mL). Insulin dose volumes are patient-dependent, but generally range from 30-100 μL of a u100 formulation. Therefore, significantly increasing the drug concentration to reduce the dose volume is not necessary for this therapeutic protein. However, commercial insulin drug products are formulated as aqueous solutions requiring refrigerated (2-8° C.) shipping and long-term storage conditions. This cold-chain requirement can limit the availability, as well as compromise the quality, of commercial insulins in third-world countries. Accordingly, there is a need for injectable insulin formulations with significantly improved thermostability which may exhibit long-term stability (i.e., storage stable for at least one year and more preferably for at least 18, 24, 30 or 36 months) at temperatures of at least 25, 30, or 35° C.

An example of the enhanced thermostability of spray-dried insulin powder relative to commercial aqueous insulin drug products is provided herein. Insulin powders were spray dried from an aqueous feed solution containing a buffer selected from glycine or histidine (ranging from ˜5-20 mM), a surfactant selected from PS20 or PS80 (ranging from ˜0.001-0.1% (w/v) and the sugar/disaccharide trehalose (from dihydrate) at pH 8.5. Due to the high solids content of the pastes, coupled with the relatively low concentration of insulin required in the final formulation (e.g. u100=3.5 mg/mL), the excipient concentration in the feed solution exceeded 99% of the total (by weight). This high ratio of excipients-to-insulin allowed the paste compositions (having ˜55-65% (w/w) solids content (powder concentration of approximately 600-780 mg/mL) and measured densities from ˜1.1-1.2 g/mL) to contain a final insulin content of approximately 3.5 mg/mL, corresponding to u100.

The Buchi B-290 mini-spray dryer used to prepare the powders had an inlet temperature of 140° C., an atomizing nozzle pressure of 60 (measured from the floating ball flow meter incorporated in the instrument), a liquid feed rate of 10% (˜3 mL/minute) and an aspirator setting of 90%. The spray dried powders were further dried (i.e., secondary dried) under vacuum to reduce the measured moisture content below approximately 1% (w/w), and then stored in glass vials and placed in a 40° C./75% RH stability chamber. The chemical stability of the insulin powders were evaluated following 123 days (˜4 months) and revealed an insulin peak purity loss of less than 2% over that storage period.

As a comparison, commercial Humulin® R insulin (aqueous solution) was stored at 40° C. in glass vials and revealed a decrease in purity of approximately 7% over 1 month as measured using the same UHPLC method used for the powders. The USP monograph for insulin injection drug products is 95-105% of label claim, indicating that the commercial drug products fall below their stability specification within one month at accelerated conditions. Therefore, the ability to prepare thermostable spray dried insulin powders for use in a therapeutic paste can improve the thermal stability of currently available commercial formulations, while still allowing for a comparable injection volume.

### Example 7

### Production of High Solids Pastes Comprising High Dose Human Proteins or Peptides

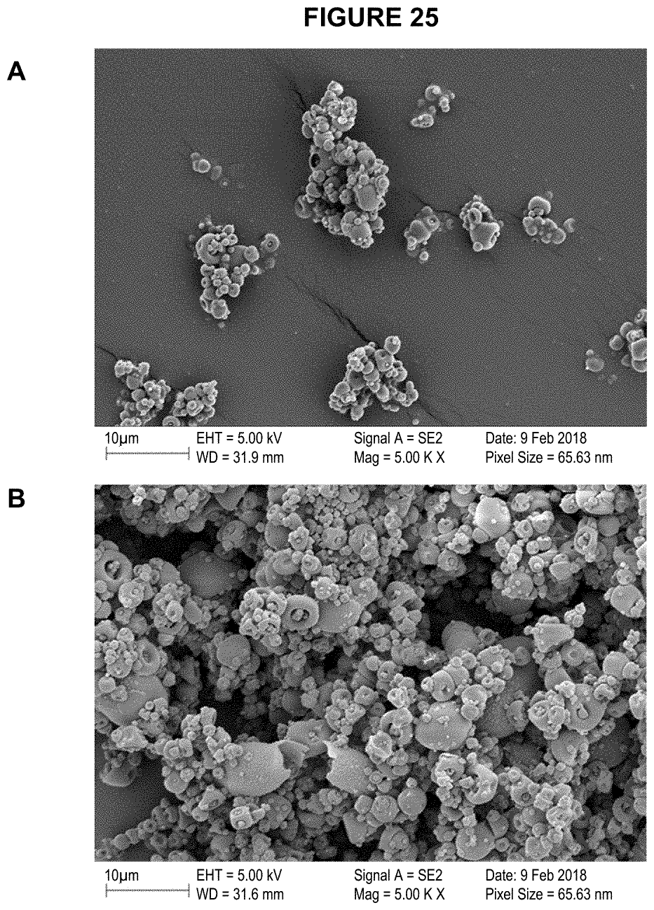

In additional studies, XeriJect™ paste formulations of high-concentration human recombinant proteins were prepared according to the spray-drying methods described in the preceding examples. In a first step, solutions of a human recombinant protein prepared and optimized according to the methods described herein (see Examples 1 and 2) were converted into dry powders by the spray-drying processes described in the preceding examples. Samples of the powder preparation were then examined by scanning electron microscopy for morphology and size distribution. Representative photomicrographs are shown in FIG. 25.

As discussed in the preceding examples, the ability to inject high concentrations and amounts of protein in a relatively low-volume subcutaneous injection of a paste formulation requires the formation of appropriately sized small and generally spherical particles in the powder used to prepare the therapeutic paste. As seen in FIGS. 25A and 25B, the protein powder prepared by the spray-drying processes described herein produced small, spherical particles with a relatively low per-particle specific surface area. Population statistics across the powder preparations (not shown) determined that the median particle size was optimal for paste formation, based on the studies described elsewhere herein (see, e.g., the preceding Examples).

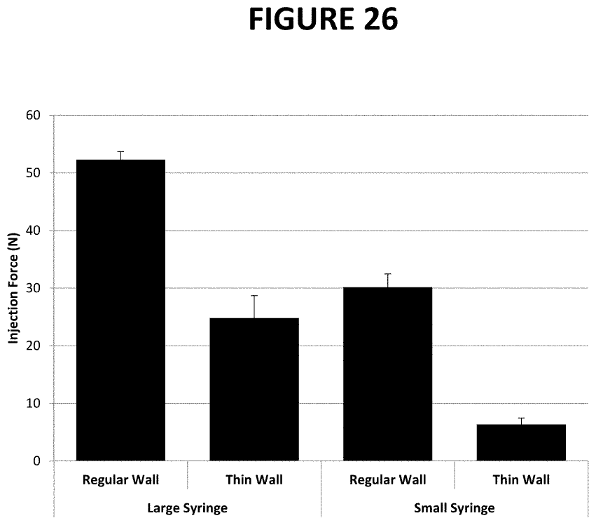

These powders were then used to prepare Xeriject™ paste formulations using the methods described above, resulting in pastes with about 45% solids content and having in excess of 300 mg/mL of protein in the pastes. A therapeutic dose of pastes having this concentration of active was calculated to be only about 150 μL. The injection force required to deliver this volume of the pastes prepared in this example were then measured as described above, in commercially available large and small syringes affixed with either regular wall or thin wall 27 G needles, at a volumetric flow rate of 30 μL per second. Results of these studies are shown in FIG. 26 and demonstrated that injection forces as low as 6N are capable with the optimal syringe/needle configuration (i.e., a small syringe with a thin wall needle).

### Example 8

### Production of High Concentration Pastes Comprising Bevacizumab

In these studies, the Xeriject™ technology was evaluated as a platform for the small volume subcutaneous delivery of a commonly used therapeutic monoclonal antibody formulation, bevacizumab (commercialized by Genentech under the AVASTIN® brand) that previously has only been capable of being administered intravenously. Bevacizumab is a human monoclonal antibody that binds Vascular Endothelial Growth Factor (VEGF), preventing interaction of VEGF with its receptor and retarding or preventing neovascularization particularly in cancerous tissues. As such, it has been approved for a number of indications in humans, including the treatment of metastatic colorectal cancer, non-squamous non-small cell lung cancers, glioblastomas and metastatic renal cell carcinomas. It is typically administered intravenously (IV) in a dosage of 10 mg/kg at an aqueous concentration of 25 mg/mL via a 30-90 minute infusion about every two weeks. The half-life of this product is about 20 days with an average clearance rate of about 0.262 L/day, depending on patient-specific parameters such as body mass, gender and tumor burden. Thus, the present inventors evaluated whether the Xeriject™ technology could be used to create formulations of bevacizumab that could be administered in a lower volume/higher dose and that would have at least similar if not more advantageous pharmacokinetics, both compared to the IV product. As described elsewhere herein, such formulations would provide significant benefits to patients and caregivers including the use of a very small volume injection which is easier to administer as a subcutaneous dose rather than via an IV route, both resulting in less discomfort to the patient.

To conduct these studies, commercially available bevacizumab drug substance was formulated into two different therapeutic paste formulations, named XJ-1 and XJ-2. The composition of the pre-spray dried aqueous solutions (mg/mL) and the approximate % wt. of the spray-dried powders are provided in Table 24.

TABLE 24
Bevacizumab Formulations Prepared Using
Xeriject ™ Technology

| | XJ-1 Formulation | | | XJ-2 Formulation | | |
|---|---|---|---|---|---|---|
| Component | Concentration | | % (w/w) | Concentration | | % (w/w) |
| Bevacizumab | 35 | mg/mL | 62.67 | 35 | mg/mL | 62.67 |
| Trehalose | 10.3 | mg/mL | 18.44 | 10.3 | mg/mL | 18.44 |
| Dihydrate | | | | | | |
| Polysorbate 80 | 0.1 | mg/mL | 0.18 | 0.1 | mg/mL | 0.18 |
| Malic Acid | 0.45 | mg/mL | 0.81 | 0.45 | mg/mL | 0.81 |
| Proline | 10 | mg/mL | 17.91 | 0 | | 0 |
| Histidine | 0 | | 0 | 10 | mg/mL | 17.91 |
| pH | 3.8 | | — | 6.0 | | — |
| Total | 55.85 | mg/mL | 100% | 55.85 | mg/mL | 100% |

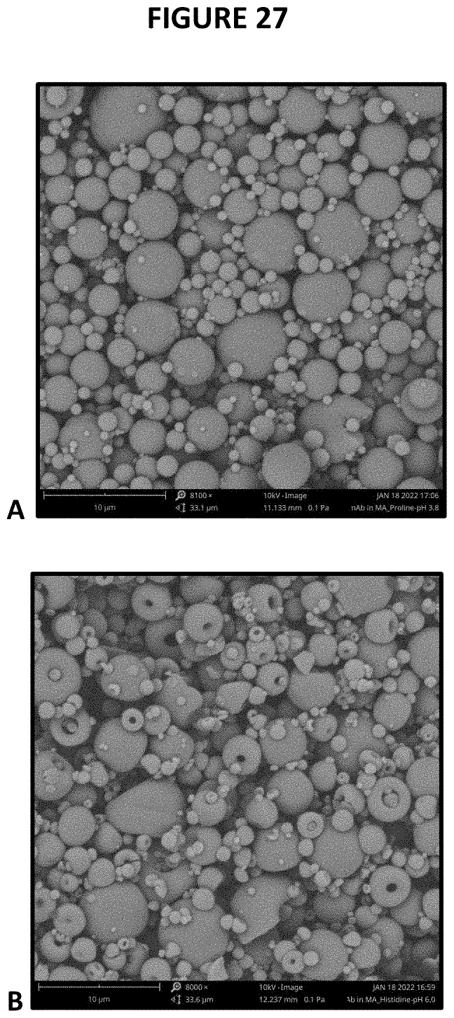

To more closely examine these powders at the particle level, a sample of powder from each of these two formulations was examined using SEM to evaluate particle size, size distribution and shape (morphology) as discussed in Examples 1-3 above. Representative photomicrographs are shown in FIG. 27, which shows that particles observed in spray-dried formulations of XJ-1 (FIG. 27A) and XJ-2 (FIG. 27B) demonstrated a range of spherical particles which had a mean size of about 3.3 μm (for XJ-1) or about 3.0 μm (for XJ-2). The XJ-2 formulation also showed a somewhat higher degree of toroidal particles compared to the XJ-1 formulation.

Based on these results, these formulations were used to prepare Xeriject™ formulations of bevacizumab for use in animal pharmacokinetic studies. For each paste, XJ-1 and XJ-2 powder was separately blended with Miglyol 812 in an HDPE container using a planetary-orbital mixer. The solids content of each of the two pastes formulation were 62% for XJ-1 and 55% for XJ-2, respectively. The measured (absorbance at 280 nm) mAb content for the two formulations were for 429 mg/mL for XJ-1 and 328 mg/mL for XJ-2.

In these studies, four groups of female Gottingen minipigs were dosed with a single 100 mg dose of one of four different bevacizumab (BmAb)formulations, either intravenously (IV) or subcutaneously (SC):

TABLE 25
Bevacizumab PK Study Design

| | | | | | Dose | |
|---|---|---|---|---|---|---|
| | | | No. of | Dose | volume/ | |
| Group | Formulation | Route | Animals | Concentration | animal | Dose |
| 1 | Avastin ® | IV | 4 | 25.87 mg/mL | 4.1 mL | 106 mg |
| 2 | Avastin | SC | 4 | 25.87 mg/mL | 4.1 mL | 106 mg |
| 3 | XJ-1 BmAb | SC | 4 | 438.67 mg/mL | 0.21 mL | 94 mg |
| 4 | XJ-2 BmAb | SC | 4 | 327.56 mg/mL | 0.22 mL | 82 mg |

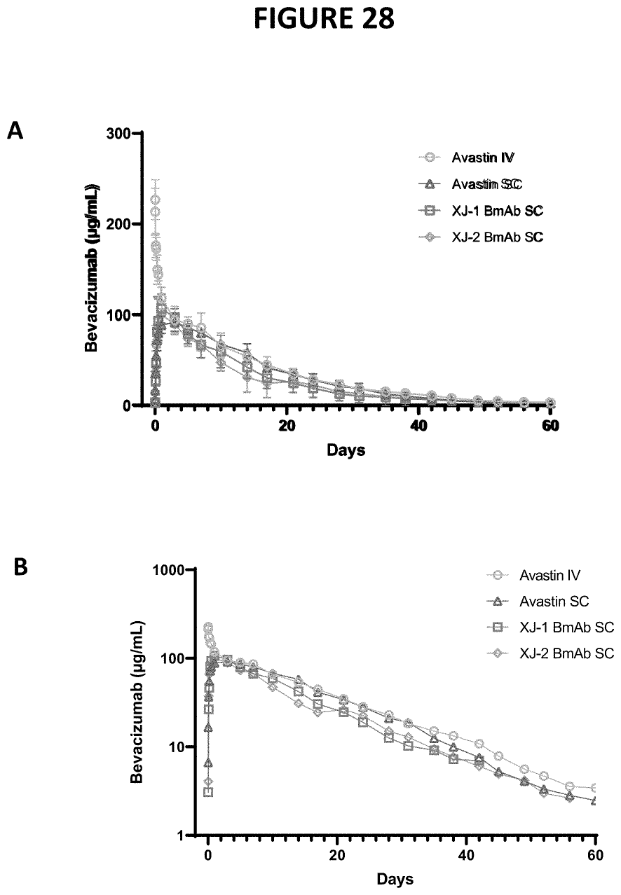

After injection of each formulation, plasma was sampled from the animals at post-injection times of 5 and 30 minutes, 2, 4, 8, 12 and 24 hours, and 3, 5, 7, 10, 14, 17, 24, 28, 31, 35, 38, 42, 45, 49, 56 and 60 days. Plasma samples were then measured for concentration of circulating bevacizumab, to allow an assessment of time to maximum plasma concentration (Tₘₐₓ), maximum concentration absorbed (Cₘₐₓ), plasma half-life (T_{1/2}), dose corrected exposure (AUC), and partial exposure (AUC). Results of these studies are shown in FIGS. 28-33.

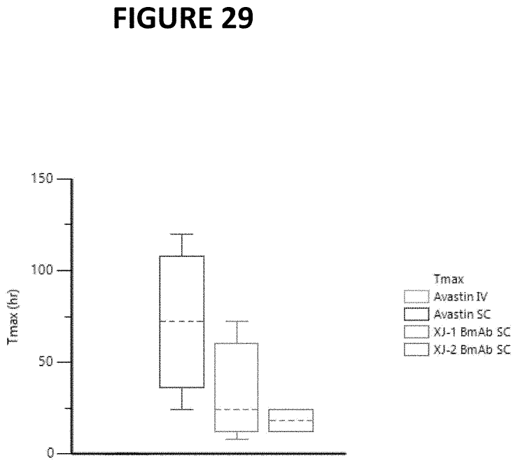

As seen in FIG. 28, the two Xeriject™ bevacizumab formulations, XJ-1 and XJ-2, showed very similar plasma concentration kinetics as the Avastin® formulation delivered subcutaneously, whether these results were plotted on a linear scale (FIG. 28A) or a semilogarithmic scale (FIG. 28B). Avastin administered intravenously showed the typical bolus effect at early time points that is commonly seen with IV-administered therapeutics. The time required to maximum plasma concentration (Tₘₐₓ) was then evaluated for each formulation, with results shown in FIG. 29. Tₘₐₓ for both Xeriject formulations was found to be shorter than the Avastin formulation administered subcutaneously (18 hours for XJ-1 and 24 hours for XJ-2, vs. 72 hours for SC Avastin), although as expected it was longer than that seen with IV-administered Avastin (0.08 hours).

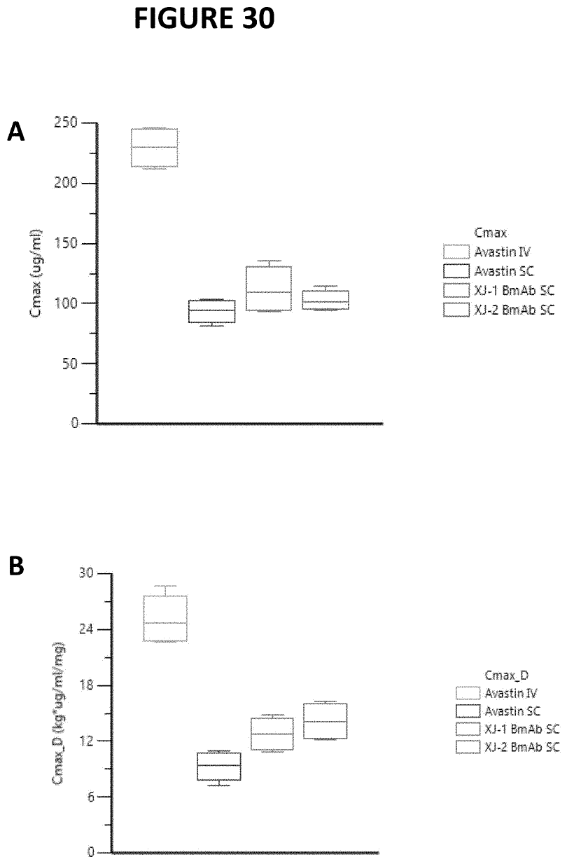

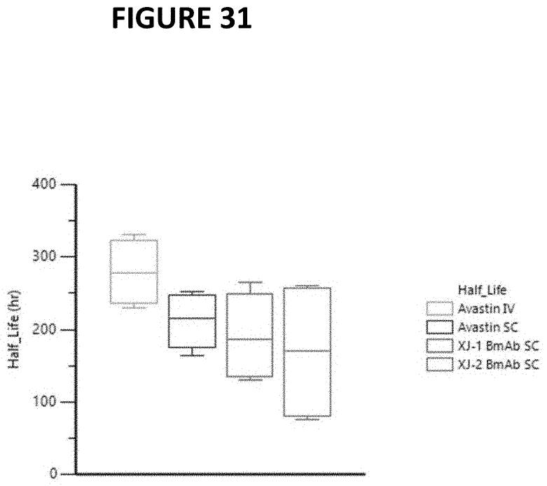

Next, the maximum absorption (Cₘₐₓ) of these various formulations was evaluated, with results shown in FIG. 30 for uncorrected Cₘₐₓ values (FIG. 30A) or Cₘₐₓ values corrected for dose and body mass of the animals (FIG. 30B). These results, together with the Tₘₐₓ results shown in FIG. 29, indicate that the Xeriject formulations of bevacizumab have more rapid absorption than Avastin administered subcutaneously, as indicated by shorter Tₘₐₓ (FIG. 29) and higher dose-corrected Cₘₐₓ (FIG. 30B) values. An assessment of the half-life for each of these formulations showed that subcutaneously administered bevacizumab formulations (both Xeriject and Avastin) demonstrated shorter half-lives than was observed for IV-administered Avastin (FIG. 31).

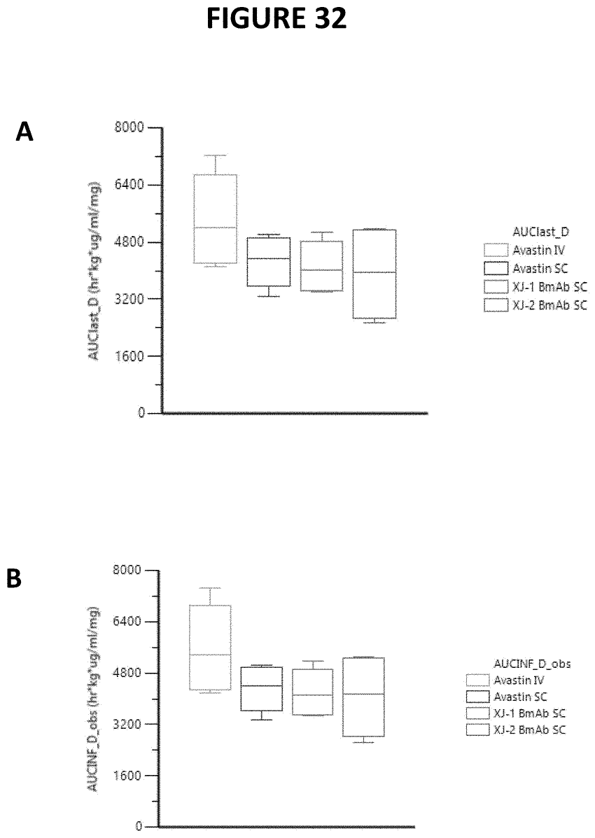

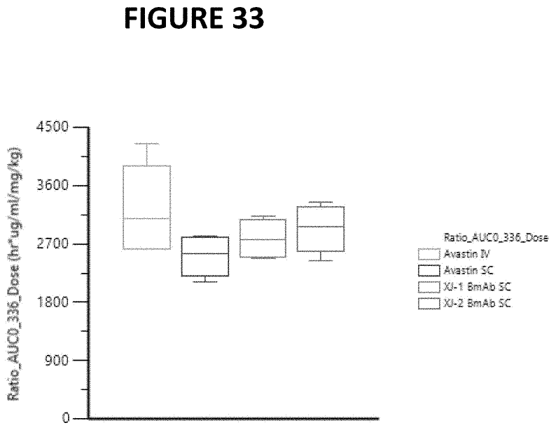

To assess total exposure of animals to the antibody, dose-corrected exposure (both AUCₗₐₛₜ and AUC_{∞}) were evaluated. Results are shown in FIG. 32 and demonstrated that dose-corrected exposure (both AUCₗₐₛₜ (FIG. 32A) and AUC_{∞} (FIG. 32B)) were similar between the Xeriject formulations and the subcutaneously administered Avastin formulation, with both assessments showing a somewhat higher exposure value in animals receiving intravenously administered Avastin. Similar results were observed when assessing dose-corrected 14-day partial exposure (AUC₀₋₃₃₆ hr; FIG. 33), although one of the Xeriject formulations (XJ-2) showed a somewhat higher partial exposure value compared to Avastin that was subcutaneously administered.

Collective results for these studies, uncorrected for dose or corrected for dose, are presented in Tables 26 and 27:

TABLE 26
Collective Pharmacokinetic Results, Xeriject vs. Avastin (Uncorrected)

| | Mean | | | | | | |
|---|---|---|---|---|---|---|---|
| | Dose | Tₘₐₓ | Cₘₐₓ | AUC₃₃₆ | AUCₗₐₛₜ | AUC_{∞} | T_{1/2} |
| Formulation | (mg) | (hr)* | (μg/mL) | (μg*hr/mL) | (μg*hr/mL) | (μg*hr/mL) | (hr) |
| Avastin ®, IV | 106 | 0.08 | 230 | 28380 | 47990 | 49271 | 227 |
| Avastin, SC | 106 | 72 | 94 | 25574 | 43336 | 44098 | 215 |
| XJ-1 BmAb | 94 | 24 | 110 | 23780 | 35001 | 35915 | 187 |
| XJ-2 BmAb | 82 | 18 | 99 | 21413 | 28603 | 29928 | 170 |

*Tₘₐₓ values are median; all other values show mean.

TABLE 27
Collective Pharmacokinetic Results, Xeriject vs. Avastin (Dose-corrected)

| | Mean | Cₘₐₓ/D | AUC₃₃₆/D | AUCₗₐₛₜ/D | AUC_{∞}/D | Bio- | Bio- |
|---|---|---|---|---|---|---|---|
| | Dose | (kg*μg/ | (hr*kg*μg/ | (hr*kg*μg/ | (hr*kg*μg/ | availability | availability |
| Formulation | (mg) | mL/mg) | mL/mg) | mL/mg) | mL/mg) | (AUCₗₐₛₜ/D) | (AUC_{∞}/D) |
| Avastin ® IV | 106 | 24.7 | 3084 | 5215 | 5357 | N/A | N/A |
| Avastin SC | 106 | 9.4 | 2553 | 4334 | 4409 | 83% | 82% |
| XJ-1 BmAb | 94 | 12.7 | 2758 | 4007 | 4112 | 77% | 77% |
| XJ-2 BmAb | 82 | 14.1 | 2968 | 3953 | 4146 | 76% | 77% |

All values show mean.

Taken together, the results presented in this example demonstrate that bevacizumab plasma concentration-time curves in minipigs are similar for two different Xeriject™ bevacizumab formulations administered subcutaneously and Avastin® administered subcutaneously, while Avastin administered intravenously has a higher peak exposure. Both bevacizumab paste formulations demonstrated more rapid absorption than Avastin administered subcutaneously, as indicated by shorter Tₘₐₓ and significantly higher dose-corrected Cₘₐₓ values for both of the Xeriject formulations. In addition, the dose-corrected exposure (AUCₗₐₛₜ and AUC_{∞}) was similar between Xeriject formulations and Avastin administered subcutaneously. The dose-corrected partial 2-week exposure (AUC₃₃₆) trended higher for the XJ-2 bevacizumab formulation compared to Avastin administered subcutaneously. Finally, the total bioavailability of Avastin administered subcutaneously and the Xeriject bevacizumab formulations was lower than observed for Avastin administered intravenously, although both were likely to still be within therapeutic dose levels. Thus, the Xeriject technology can be used to prepare high concentration formulations of bevacizumab that can be injected subcutaneously at lower volumes and less frequently than those currently used to deliver Avastin intravenously, while still delivering therapeutic levels of antibody that appears to be well-tolerated and rapidly absorbed in subject animals.

### Example 8

### Nonclinical Evaluation of High Solids Pastes Comprising Insulin

To evaluate the pharmacokinetic (PK) and pharmacodynamic (PD) profiles of insulin pastes, two paste formulations (XJ-6 and XJ-8) were prepared as described in Example 6 and evaluated in Yucatan minipigs. Both XJ-6 and XJ-8 were formulated to have an insulin content corresponding to u200 (7.5 mg/mL). Insulin powders were prepared by spray drying, with a feed solution containing a total solids-loading (dissolved material) of 50 mg/mL and comprising recombinant human insulin (0.52 mg/mL), trehalose (from dihydrate; 49.0 mg/mL), histidine (0.3 mg/mL) PS80 (0.01 mg/mL), EDTA (0.1 mg/mL) and pH adjusted to 9.0 (±0.1) using NaOH and/or HCl.

This solution was filtered (0.2 μm PVDF membrane) and spray dried with a BUCHI B-290 instrument at the following settings: inlet temp=140° C./liquid feed rate=8%/nozzle gas pressure ball meter reading=60 mm/aspirator setting=90%. The powder was secondary dried under vacuum to reduce the moisture content of the powder to <2% (w/w). The powder was blended with either pure Miglyol 812 (XJ-6) or Miglyol 812 containing 1% (v/v) PS80 (XJ-8). The solids contents of XJ-6 and XJ-8 were 57% and 56%, corresponding to solids concentrations of approximately 680 mg/mL and 670 mg/mL, respectively.

These test articles (XJ-6 (XeriJect-6) and XJ-8 (XeriJect-8)) were evaluated for pharmacokinetics (PK) and pharmacodynamics (PD) (changes in blood glucose) in the Yucatan minipig and compared to the commercial product Humulin R (u100). Each formulation was administered by subcutaneous injection to 6 male Yucatan minipigs at a dose of 0.5 U/kg insulin (0.017 mg/kg insulin).

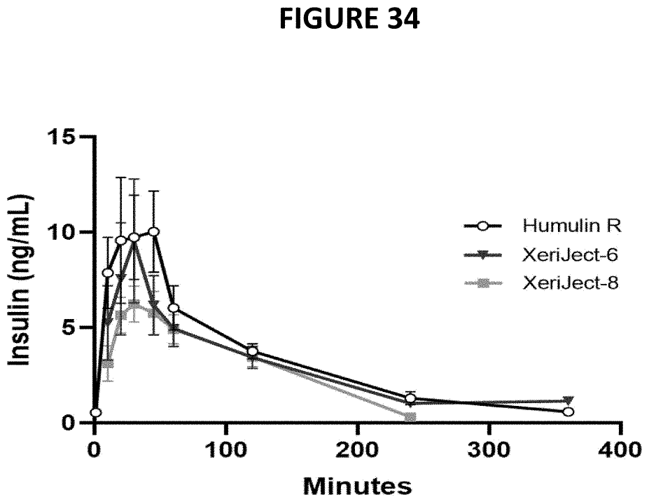

The insulin pharmacokinetic profiles of all three formulations were similar, although insulin exposure was lower with XJ-6 as shown in FIG. 34. Administration of 0.5 U/kg Humulin R (3.5 mg/mL insulin) SC to minipigs produced plasma insulin exposure with a mean (±SD) Cₘₐₓ of 13±3 ng/mL and mean (±SD) AUCₗₐₛₜ of 1175±144 ng*min/mL. The median Tₘₐₓ was 30 min (range: 10 min to 45 min) and mean (±SD) half-life was 79±12 min. Administration of 0.5 U/kg XJ-6 (17.4 mg/mL insulin) SC to minipigs produced similar, though slightly lower insulin exposure compared to Humulin R with a mean (±SD) Cₘₐₓ of 10±3 ng/mL and a mean (±SD) AUCₗₐₛₜ of 902±139 ng*min/mL. The median tₘₐₓ (30 min, range: 30 min to 60 min) was similar to Humulin R and the half-life (129 min) was 63% longer. XJ-8 (17.4 mg/mL insulin) administration of 0.5 U/kg SC to minipigs produced lower insulin exposure compared to Humulin R with a mean (±SD) Cₘₐₓ of 7.0±1.1 ng/mL and a mean (±SD) AUCₗₐₛₜ of 643±92 ng*min/mL. The median Tₘₐₓ (30 min, range: 20 min to 45 min) and half-life (67 min) were similar to Humulin R.

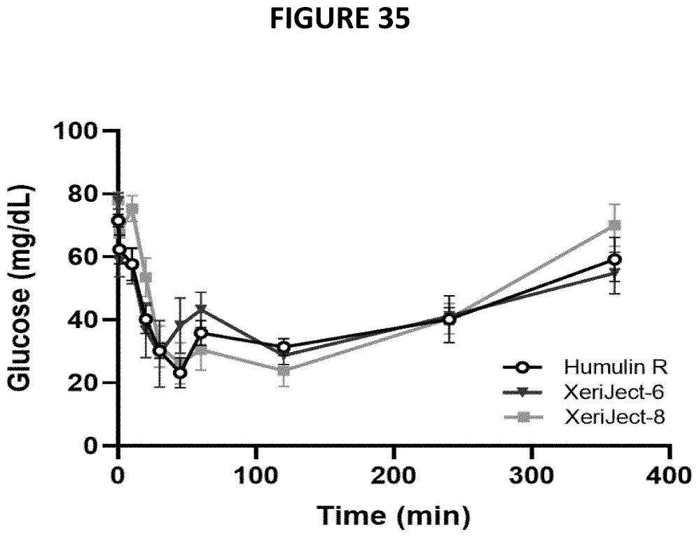

Administration of Humulin R, XJ-6 and XJ-8 produced similar PD responses by rapidly decreasing blood glucose levels as demonstrated in FIG. 35. Humulin R decreased blood glucose to a mean (±SD) of 21±4 mg/dL from a mean (±SD) baseline of 72±2 mg/dL by a median time of 38 min (range: 30 min to 45 min). XeriJect-6 decreased blood glucose levels to a mean (±SD) of 14±6 mg/dL from a mean (±SD) baseline of 78±3 mg/dL by a median time of 75 min (range: 20 min to 360 min). XeriJect-8 decreased blood glucose levels to a mean (±SD) of 17±6 mg/dL from a mean (±SD) baseline of 78±3 mg/dL by a median time of 83 min (range: 30 min to 120 min). Some instances of hypoglycemia occurred following insulin administration and animals were treated with oral administration of 50% dextrose. Pharmacodynamic effects of test articles on glucose levels shown here are only approximate for minipigs provided dextrose for treatment of hypoglycemia.

### Example 9

### Production, Characterization and Preparation of an Injectable Paste Comprising Immune Globulin G (IgG)

The following example describes the preparation of injectable paste formulations containing high concentrations of the polyclonal antibody (pAb) immune globulin G (IgG). The solids phase in this example comprised an IgG powder prepared by spray drying an aqueous feed solution having a solids-loading of approximately 51 mg/mL, of which 40 mg/mL is the IgG protein (corresponding to over 78% (wt.) of the total solids-loading). Prior to the final feed solution preparation, IgG was buffer-exchanged against an aqueous solution to yield a final feed solution composition listed in Table 28.

TABLE 28
Formulation Feed Solution Composition
for IgG Powder Preparation

| Composition | Concentration | % wt. |
|---|---|---|
| IgG | 40 mg/mL | 78.66 |
| Trehalose (from Dihydrate) | 10.3 mg/mL | 20.26 |
| Polysorbate 20 | 0.1 mg/mL | 0.20 |
| Lactic Acid | 0.45 mg/mL | 0.88 |
| pH | 3.8 | — |
| Total | 50.85 | 100 |

This formulation feed solution was spray dried using BUCHI B-290 spray dryer parameters and conditions shown below in Table 29.

TABLE 29
Spray Dryer Parameters and Conditions
for IgG Powder Preparation

| Parameter | Values | |
|---|---|---|
| Inlet temperature | 80° C. | |
| Aspirator | 70% | (28 m³/h) |
| Feed flow rate | 10% | (3 mL/min) |
| Nozzle flow rate | 60 mm | (742 L/h) |

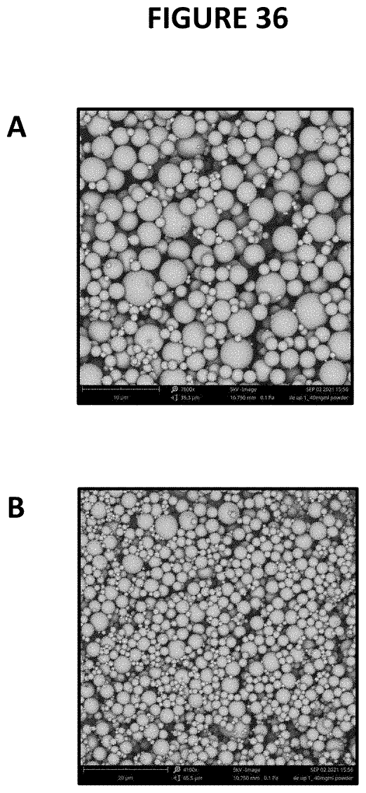

The spray dried powder was secondary dried under reduced pressure to lower moisture content (<1% (w/w)) and then evaluated by scanning electron microscopy (SEM) to examine the particle morphology. As seen in FIGS. 36A and 36B, the particles exhibited a generally spherical shape with a relatively smooth surface, minimal-to-no observed surface pitting (dimpling), and a moderately polydisperse size distribution (span ˜2.0).

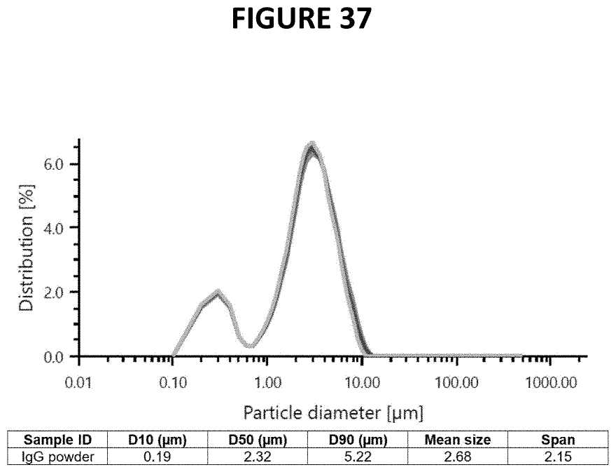

Next, the particle size and particle size distribution of the IgG powder was determined by laser diffraction (where the sample was dispersed in a non-solvent (e.g., propyl alcohol) with continuous sample sonication to disrupt powder agglomerates). As shown in FIG. 37, a small particle population having a D90<10 μm with a moderate polydisperse (span ˜2) and bi-modal distribution where there is a defined population of fine particles (<1 μm) and a population of larger (1-10 μm) particles. It is noted that under the conditions described in this example a relatively bi-modal particle size distribution was observed. However, changes to the formulation and/or process parameters and/or equipment (e.g., larger scale spray dryer, etc.) and/or characterization method can shift the observed particle size distribution to generally unimodal or multi-modal (e.g., bimodal, trimodal, etc.) profiles that remain suitable for preparing high solids concentration pastes.

Following characterization by SEM, laser diffraction, and moisture content analysis (data not shown), a paste was prepared by blending the powder with Miglyol 812 N (using a planetary-orbital mixer) to a solids content of 65%. The density of the powder measured by helium pycnometry was approximately 1.2 g/mL, corresponding to a solids concentration of approximately 780 mg/mL. The solids-loading of the feed solution, particularly the weight-percent of the protein in the feed solution as a percentage of total solids-loading (˜78% w/w), may be translated to the approximate weight-percent of IgG in the spray dried powder (˜78% (w/w)), which may then be used to determine the approximate protein concentration in the paste (˜600 mg/mL).

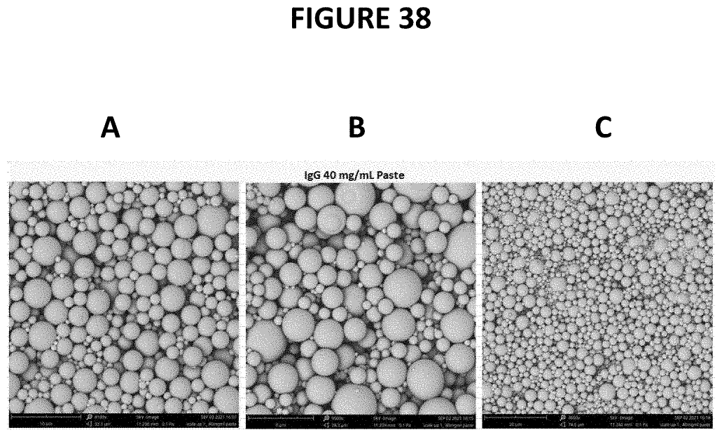

The IgG paste was also imaged by SEM to examine any changes in the morphology and/or size distribution of the particles (post-blending with Miglyol) and to observe the particle packing in the paste. As shown in FIGS. 38A, 38B, and 38C, after the preparation of the IgG paste the morphology and size distribution of the particles comprising the solids phase of the paste remained relatively unchanged. The SEM analysis of the IgG paste indicated good particle packing/arrangement arising from the highly concentrated solids phase of the paste, which imparts the semi-solid and viscoelastic properties of the paste and may sterically inhibit the particles from settling over time under storage conditions and periods relevant to a pharmaceutical drug product.

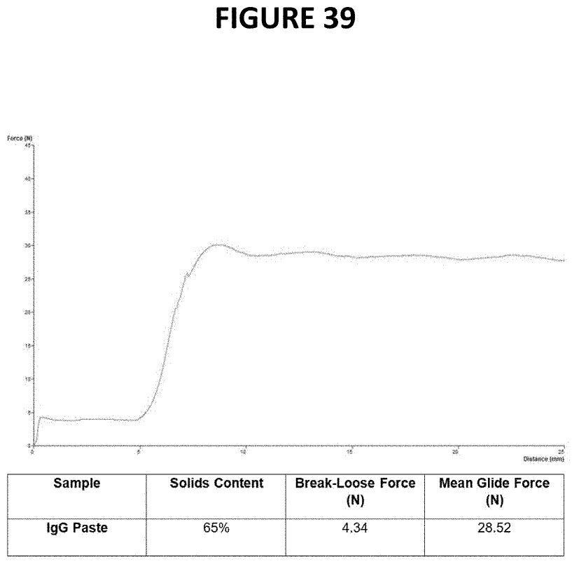

Finally, to evaluate the injectability of the high solids concentration IgG paste, 1 mL of the paste was loaded into cyclic olefin copolymer (COC) syringes (Schott) having a barrel internal diameter of 5.0 mm and delivered through a 25-gauge, ultra-thin wall, 6 mm needles. FIG. 39 provides the injection force profile (plotted as force in Newton (N; Y-axis) versus plunger distance traveled in millimeters (mm, X-axis)) of the 65% solids content IgG paste measured using a TA.XT Plus model texture analyzer (Stable Micro Systems). The measured injection force (mean glide force) was below 30 N at a volumetric flow rate of 31 μL/sec, as measured by a texture analyzer.

Thus, this example demonstrates that paste compositions having both a high solids concentration (>700 mg/mL) and a high protein concentration (>500 mg/mL) can be prepared and delivered through syringes and needles relevant to intracutaneous injection with moderate injection forces.

Together with those of the preceding examples, these results indicate that the paste formulations of high concentration therapeutic proteins that are provided by the present invention are useful in delivering controlled- or sustained-release depots of therapeutic proteins, including those of relatively high molecular weights, in small volumes subcutaneously in a way that improves the patient experience from that obtained with higher volume administration of aqueous formulations of recombinant human proteins.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Hence, in addition to those specifically described herein, other suitable embodiments of the invention will be readily apparent to one of ordinary skill in the art based upon the foregoing description and examples, and upon knowledge generally available in the relevant arts. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All references cited herein, including U.S. patents and published patent applications, international patents and patent applications, and journal references or other publicly available documents, are incorporated herein by reference in their entireties to the same extent as if each reference had been specifically cited for the portion or portions of such reference applicable to the section of this application to which it is relevant.

1. A composition comprising a paste having a solids concentration of greater than about 350 mg/mL, said paste comprising one or more active pharmaceutical ingredients, one or more pharmaceutically acceptable excipients, and one or more non-solvent fluids, wherein said paste is capable of being injected subcutaneously, intracutaneously or intramuscularly into an animal in a volume of 3 ml or less at a flow rate of at least 30 μL/s using a commercially available needle/syringe combination.

2. The composition of claim 1, wherein the solids concentration of the paste is from about 350 mg/mL to about 850 mg/mL.

3-5. (canceled)

6. The composition of claim 1, wherein the paste has a relative content of active pharmaceutical ingredient of from about 20% to about 70%.

7-11. (canceled)

12. The composition of claim 1, wherein said active pharmaceutical ingredient is selected from the group consisting of a peptide, a protein, and a small molecule therapeutic.

13. The composition of claim 1, wherein said active pharmaceutical ingredient is a peptide or protein therapeutic.

14. The composition of claim 13, wherein said peptide or protein therapeutic is selected from the group consisting of an enzyme, an antithrombin agent, a thrombolytic agent, a peptide hormone, a bone-active peptide, a diabetic-active peptide, an antibody, a non-antibody antineoplastic agent, a fertility agent, and an immunosuppressive agent.

15. The composition of claim 14, wherein said enzyme is selected from the group consisting of dornase alpha, velaglucerase alpha, taliglucerase alpha, asparaginase, glucarpidase, asfotase alpha, elosulfase alpha, sebelipase alpha, sacrosidase, and pegloticase.

16. The composition of claim 14, wherein said antithrombin agent is selected from the group consisting of lepirudin, bivalirudin, defibrotide, and sulodexide.

17. The composition of claim 14, wherein said a thrombolytic agent is selected from the group consisting of reteplase, anistreplase, tenecteplase, streptokinase, and urokinase.

18. The composition of claim 14, wherein said peptide hormone is selected from the group consisting of cosinotropin, chorionic gonadotropin, and somatotropin.

19-20. (canceled)

21. The composition of claim 14, wherein said bone-active peptide is calcitonin.

22. (canceled)

23. The composition of claim 14, wherein said diabetic-active peptide is selected from the group consisting of insulin, pramlintide, glucagon, and analogues thereof.

24. The composition of claim 23, wherein said diabetic-active peptide is insulin or an analogue thereof.

25. The composition of claim 24, wherein said insulin analogue is selected from the group consisting of insulin lispro, insulin glargine, insulin aspart, insulin detemir, and insulin glulisine.

26-27. (canceled)

28. The composition of claim 23, wherein said diabetic-active peptide or protein is glucagon or an analogue thereof.

29. The composition of claim 28, wherein said glucagon analogue is dasiglucagon.

30. (canceled)

31. The composition of claim 14, wherein said antibody is a monoclonal antibody or a fragment thereof.

32. The composition of claim 31, wherein said monoclonal antibody is selected from the group consisting of cetuximab, trastuzumab, bevacizumab, rituximab, obinutuzumab, gemtuzumab, canakinumab, ipilimumab, daratumumab, vedolizumab, ustekinumab, siltuximab, ramucirumab, pembrolizumab, ofatumumab, nivolumab, mepolizumab, brodalumab, pertuzumab, denosumab, golimumab, belimumab, raxibacumab, blinatuomab, dinutuximab, and ibritumomab.

33. The composition of claim 14, wherein said non-antibody antineoplastic agent is selected from the group consisting of leuprolide, denileukin diftitox, aldesleukin, asparaginase, pegasparagase, interferon beta, afibercept, lenograstim, and sipuleucel-T.

34. (canceled)

35. The composition of claim 14, wherein said immunosuppressive agent is selected from the group consisting of etanercept, peginterferon alpha, an interferon alpha, filgrastim, pegfilgrastim, sargramostim, anakinra, an interferon beta, an interferon gamma, adalimumab, infliximab, basiliximab, muromonab, efalizumab, daclizumab, abatacept, rilonacept, belatacept, natalizumab, blintumomab, ustekinumab, and human immune globulin.

36. The composition of claim 13, wherein said peptide or protein therapeutic is a recombinant peptide or protein.

37. (canceled)

38. The composition of claim 12, wherein said small molecule therapeutic is selected from the group consisting of epinephrine or an analogue thereof, benzodiazepines, catecholamines, “triptans,” sumatriptan, novantrone, a chemotherapy small molecule, a corticosteroid small molecule, an immunosuppressive small molecule, an anti-inflammatory small molecule a small molecule used to treat neurological disorders, a small molecule used to treat cancer, a statin, taxol and other taxane derivatives, a small molecule used to treat tuberculosis, a small molecule anti-fungal agent, a small molecule anti-anxiety agent, a small molecule anti-convulsant agent, a small molecule anti-cholinergic agent, a small molecule β-agonist drug, a small molecule mast cell stabilizer, a small molecule agent used to treat allergies, a small molecule anesthetic agent/anti-arrhythmic agent, a small molecule antibiotic agent, a small molecule anti-migraine agent, and a small molecule anti-histamine drug, and a salt or analogue thereof.

39. The composition of claim 38, wherein said small molecule therapeutic is a benzodiazepine.

40. The composition of claim 38, wherein said small molecule therapeutic is epinephrine or an analogue thereof.

41. The composition of claim 1, wherein said one or more pharmaceutically acceptable excipients are selected from the group consisting of a saccharide, a surfactant, an amino acid and a buffering agent.

42. The composition of claim 41, wherein said saccharide is selected from the group consisting of trehalose, dextrose, sucrose, mannose and fructose.

43. The composition of claim 41, wherein said one or more pharmaceutically acceptable excipients are selected from the group consisting of polysorbate 20, polysorbate 80, Miglyol 810, Miglyol 812, and Miglyol 840.

44. The composition of claim 41, wherein said amino acid is a naturally occurring amino acid selected from the group consisting of proline, cysteine, tryptophan, phenylalanine, arginine, and histidine.

45-46. (canceled)

47. The composition of claim 41, wherein said buffering agent is selected from the group consisting of histidine, citrate, succinate and lactate.

48. The composition of claim 1, wherein said non-solvent fluid is triacetin or Miglyol 812.

49. A method of treating, preventing, ameliorating, or diagnosing a disease or disorder in an animal or human suffering from or predisposed to said disease or disorder, comprising injecting the composition of claim 1 into said animal or human subcutaneously, intracutaneously or intramuscularly.

50. The method of claim 49, wherein said method comprises:
preparing a combination device comprising a needle attached to a syringe, said device comprising said composition in the barrel of said syringe in a volume sufficient to deliver a therapeutic dose of at least one active pharmaceutical ingredient into said animal or human;
introducing the needle of said syringe-needle combination into the cutaneous, subcutaneous or muscle layers said animal or human; and
moving a plunger of a syringe to dispense paste from a reservoir of the syringe through a lumen of the needle attached to the syringe, the reservoir having an internal first transverse dimension that is larger than an internal second transverse dimension of the lumen, where the second transverse dimension is between 0.1 and 0.9 mm, thereby dispensing the paste through the needle into the animal or human;
wherein the paste has a solids concentration of greater than 350 mg/mL; and
wherein the paste is dispensed-at a flow rate of greater than 30 μL/s.

51. The method of claim 50, further comprising coupling the needle to the reservoir via a Luer fitting.

52. The method of claim 50, where the flow rate of the paste is substantially linearly proportional to the rate of plunger movement.

53. The method of claim 50, where the first transverse dimension is 3 to 40 times larger than the second transverse dimension.

54. The method of claim 50, where the first transverse dimension is between 1 and 5 mm.

55. (canceled)

56. The method of claim 50, where the needle has a size of 18 Gauge or smaller.

57. The method of claim 56, where the needle has a size of 23 Gauge or smaller.

58. The method of claim 56, where the needle has a size of 27 Gauge.

59. (canceled)

60. The method of claim 50, where the injected volume of paste is between 10 μL and 3000 μL.

61-62. (canceled)

63. The method of claim 50, where the paste has a solids concentration of between 350 and 850 mg/mL.

64. The method of claim 50, where the paste has a solids content of between 1% and 99%.

65. The method of claim 50, where the paste has a solids content of between 30% and 40%.

66. The method of claim 50, where the paste has a density of between 1.0 and 1.5 g/mL.

67. The method of claim 49, wherein said disease or disorder is selected from the group consisting of a diabetic disease or disorder, an inflammatory disease or disorder, a neurological disease or disorder, a cancer, an infectious disease, a bacterial disease, a fungal disease, a viral disease, and a disease, disorder or condition involving inflammatory, neurological, osteological, gastrointestinal, circulatory, cardiovascular, skin, muscular or developmental signs or symptoms.
